(19) United States
(12) Patent Application Publication
(10) Pub. No.: US 2025/0352409 A1
(43) Pub. Date: Nov. 20, 2025

(54) BODY-CONFORMABLE ABSORBENT ARTICLES WITH AN ELASTIC BACKSHEET

(71) Applicant: THE PROCTER & GAMBLE COMPANY, Cincinnati, OH (US)

(72) Inventors: Frederick Michael LANGDON, Cincinnati, OH (US); Urmish Popatlal DALAL, Milford, OH (US); Timothy Ian MULLANE, Union, KY (US)

(21) Appl. No.: 19/199,567

(22) Filed: May 6, 2025

Related U.S. Application Data
(60) Provisional application No. 63/647,289, filed on May 14, 2024.

(51) Int. Cl. A61F 13/53; A61F 13/47; A61F 13/514

(52) U.S. Cl. CPC A61F 13/53; A61F 13/51464; A61F 2013/4708; A61F 2013/530817

(57) ABSTRACT

Disposable absorbent articles having a topsheet; a backsheet having an elastic layer and a constricting layer; and an absorbent core structure having an open-celled absorbent foam material disposed between the topsheet and the backsheet. The absorbent foam material includes a plurality of discrete foam pieces, wherein the discrete foam pieces are separated from neighboring pieces by a Relaxed Gap of from about 10 microns to about 280 microns, as measured according to the SEM Imaging Method. The articles exhibit an OD Full Product Force Threshold at 15 mm extension of greater than 7.2 N, as measured according to the Simple Tensile Method.

### CROSS REFERENCE TO RELATED APPLICATIONS

This application claims the benefit, under 35 U.S.C. § 119 (e), to U.S. Provisional Application No. 63/647,289, filed May 14, 2024, the entire disclosure of which is fully incorporated by reference herein.

### FIELD OF THE INVENTION

The present disclosure relates to body-conformable absorbent articles with an elastic backsheet, in particular body-conformable absorbent articles with an elastic backsheet that can provide fluid acquisition/distribution performance and resists backsheet tearing during removal.

### BACKGROUND OF THE INVENTION

Absorbent articles are widely used among consumers, e.g., diapers, training pants, feminine pads, adult incontinence pads, etc. Generally, absorbent articles such as these comprise a topsheet and a backsheet, with an absorbent core structure disposed therebetween. These absorbent articles are designed to absorb and retain liquids and other discharges from the human body to prevent body and garment soiling. To absorb fluid effectively without leakage, absorbent articles should conform closely to a wearer's body to intercept discharged fluid moving along skin surfaces and prevent it from escaping the absorbent article.

An absorbent article that can conform and move with the body can be created by bidirectionally stretching (or “activating”) the article until the absorbent core structure is fractured into discrete pieces (or “cells”) of absorbent material. The backsheet and topsheet (to which the discrete pieces may be attached) undergo plastic deformation during the activation process and do not fully recover to their relaxed dimensions (length and width), creating relatively wide gaps (i.e., greater than about 280 microns) between the pieces of absorbent material. As a result, highly flexible segments are created that can significantly lower the flexural bending rigidity of the entire absorbent article. While this structure can create a highly flexible, body-conformable absorbent article that allows for fast fluid acquisition, the relatively wide gaps formed between the pieces of absorbent material can create an undesirable visual impression that the absorbent article is too thin or flimsy and may not provide a sufficient level of leakage protection. In addition, if the gap size is too large, there is a tendency for free fluid droplets to accumulate in the channels between the pieces of absorbent material and/or on the topsheet (as demonstrated by high fluid rewet values) and can lead to an uncomfortable wet feeling during use. Also, if the gap size between adjacent cells becomes too large, fluid may no longer distribute between the cells in the X-Y plane of the absorbent core structure. As a result, the cells within a localized area can load to capacity and become saturated as fluid distribution within the discreet cells is disrupted and the volume of fluid can no longer be removed from the localized area. This localized saturation can lead to a backup of fluid during additional loading and can be reflected in relatively high rewet values as measured according to the Acquisition Time and Rewet Method.

When the absorbent articles described above contain an elastic backsheet, the discrete pieces of absorbent material can be brought closer together after activation, thus decreasing the gap size and re-establishing fluid continuity and distribution between the pieces of absorbent material. However, a highly elastic backsheet can be strained to relatively high levels at relatively low forces that are above the yield force of the absorbent article and, particularly, the backsheet. During removal of an absorbent article from an undergarment, a highly elastic backsheet will want to stay attached to the undergarment and will continue to stretch in an uncontrolled manner, causing the backsheet to tear and/or the absorbent article to burst. This can be highly undesirable because a torn backsheet can expose the contents of the absorbent core and create a hygienic problem. In addition, the pieces of absorbent material can shear from the surfaces to which they are attached (i.e., the topsheet and/or the backsheet) during removal, causing a loss of product integrity.

Thus, there is a need for an improved absorbent article that can conform to the body while still maintaining fluid acquisition and distribution and does not tear and/or lose integrity during removal.

### SUMMARY OF THE INVENTION

The present disclosure solves the problems of highly conformable absorbent articles that have high rewet values and tear and/or lose integrity during removal by providing an elastic backsheet that can bring the pieces of absorbent material relatively close together and create a gap size of about 280 microns or less. The absorbent article may also have a force threshold at low extensions (i.e., 10-15 mm) that is greater than the peel force of the article during removal from an undergarment, thus reducing the risk of backsheet tearing and/or detachment of the pieces of absorbent material from the topsheet or backsheet.

Described herein is a disposable absorbent article comprising: a topsheet; a backsheet; and an absorbent core structure comprising an open-celled absorbent foam material disposed between the topsheet and the backsheet. The backsheet comprises an elastic layer and a constricting layer. The absorbent foam material comprises a plurality of discrete foam pieces, wherein the discrete foam pieces are separated from neighboring pieces by a Relaxed Gap of about 280 microns or less, as measured according to the SEM Imaging Method, and by a Stretched Gap of from about 500 microns to about 2.5 mm, as measured by the Stretched Gap Measurement Method.

Also described herein is a disposable absorbent article comprising: a longitudinal axis and a lateral axis; a topsheet; a backsheet; and an absorbent core structure comprising an open-celled absorbent foam material disposed between the topsheet and the backsheet. The backsheet comprises an elastic layer and a constricting layer. The topsheet and the backsheet each comprise a first plurality of lines of deformation extending in a first direction and a second plurality of lines of deformation extending in a second direction. The absorbent core structure comprises a plurality of discrete foam pieces arranged along the lines of deformation and at least a portion of the backsheet that is located in the first and second plurality of lines of deformation has a plurality of micro-wrinkles.

Also described herein is a disposable absorbent article comprising: a topsheet; a backsheet comprising an elastic layer and a constricting layer; and an absorbent core structure comprising an open-celled absorbent foam material disposed between the topsheet and the backsheet. The absorbent foam material comprises a plurality of discrete foam pieces, wherein the discrete foam pieces are separated from neighboring pieces by a Relaxed Gap of from about 10 microns to about 280 microns, as measured according to the SEM Imaging Method. The article exhibits an OD Full Product Force Threshold at 15 mm extension of greater than 7.2 N, as measured according to the Simple Tensile Method.

### BRIEF DESCRIPTION OF THE DRAWINGS

FIG. 1 is a plan view of an absorbent article in the form of a feminine hygiene pad, wearer-facing surface facing the viewer, illustrating an example of a pattern of bidirectional deformation.

FIG. 2 is a plan view of an absorbent article in the form of a feminine hygiene pad, wearer-facing surface facing the viewer, illustrating various other features.

FIGS. 3A and 3B are illustrations showing exemplary panty fastening adhesive patterns according to the present disclosure.

FIGS. 4A-4C are schematic plan views of non-limiting examples of patterns of arrangements of bonding regions between a topsheet and underlying components of an absorbent article.

FIG. 4D is a schematic plan view of another example of a pattern of adhesive deposit to form a bonding region between a topsheet and underlying components of an absorbent structure.

FIG. 5A is a schematic lateral cross section of the article shown in FIG. 1, taken through line 5A-5A in FIG. 1.

FIG. 5B is an enlarged view of the portion of FIG. 5A shown encircled in circle 5B, in one possible example.

FIG. 5C is an enlarged view of the portion of FIG. 5A shown encircled in circle 5B, in another possible example.

FIG. 6A is a cross-sectional view of a first embodiment of a backsheet according to the present disclosure.

FIG. 6B is a cross-sectional view of a second embodiment of a backsheet according to the present disclosure.

FIG. 7 is a plan view of another example of an absorbent article in the form of a feminine hygiene pad, wearer-facing surface facing the viewer, illustrating another example of a pattern of bidirectional deformation.

FIG. 8 is an enlarged view of arrows indicating directions of lines of stretch and of deformation along an x-y plane, relative longitudinal and lateral axes of an absorbent structure.

FIG. 9 is a plan view of another example of an absorbent article in the form of a feminine hygiene pad, wearer-facing surface facing the viewer, illustrating particular features.

FIG. 10 is a simplified schematic side-view depiction of components arranged for a process for deforming a composite web.

FIG. 11 is a perspective view of an example of a pair of deforming rollers.

FIG. 12 is a view of engaging features of portions of an example of a pair of deforming rollers.

FIG. 13 is a closer view of the features shown in FIG. 12, shown acting upon a web.

FIG. 14 is a schematic cross section view (along a z-direction plane) of an example of a deformed web.

FIG. 15 is a view along the machine direction, of another example of a pair of deforming rollers.

FIG. 16 is a view along the machine direction, of another example of a pair of deforming rollers.

FIG. 17A is a view along the machine direction, of another example of a pair of deforming rollers.

FIG. 17B is a view along the machine direction, of another example of a pair of deforming rollers.

FIG. 18A is a view along the machine direction, of another example of a pair of deforming rollers.

FIG. 18B is a view along the machine direction, of another example of a pair of deforming rollers.

FIG. 19A is a SEM photomicrograph showing a tilted cross-sectional view of a portion of an elastic layer that has been activated, specifically showing a portion of the elastic layer located outside of a line of deformation.

FIG. 19B is a SEM photomicrograph showing a tilted cross-sectional view of a portion of an elastic layer that has been activated, specifically showing a portion of the elastic layer located within a line of deformation.

FIG. 20A is a SEM photomicrograph showing a tilted cross-sectional view of an absorbent article as described herein after activation, garment-facing surface facing the viewer, showing a plurality of lines of deformation.

FIG. 20B is a magnified (35×) SEM photomicrograph cross-sectional view of the portion of FIG. 20A shown in square 20B.

FIG. 21A is a SEM photomicrograph showing a tilted cross-sectional view of an absorbent article as described herein after activation, garment-facing surface facing the viewer, showing a plurality of lines of deformation.

FIG. 21B is a magnified (300×) SEM photomicrograph cross-sectional view of the portion of FIG. 21A shown in square 21B.

FIG. 22 is a SEM photomicrograph showing a tilted top view of an absorbent article as described herein that has been activated, specifically showing a plurality of lines of deformation in the backsheet having one or more macro-wrinkles.

FIG. 22A is a magnified SEM photomicrograph cross-sectional view of a portion of the absorbent article of FIG. 21A.

FIG. 22A1 is a magnified SEM photomicrograph cross-sectional view of the portion of FIG. 22A shown in square 22A1 located within a line of deformation.

FIG. 22A2 is a magnified SEM photomicrograph cross-sectional view of the portion of FIG. 22A shown in square 22A2 located outside of a line of deformation.

FIG. 23 is a graph of the elastic hysteresis behavior of Film C when subjected to 100% strain and examined for elastic hysteresis response.

FIG. 24 is a graph of the elastic hysteresis behavior of Film D when subjected to 100% strain and examined for elastic hysteresis response.

FIG. 25 is a schematic depiction of equipment used in the Conformability Force Measurement Method, described herein.

FIG. 26 is a schematic depiction of equipment used in the Capillary Work Potential via Pore Volume Distribution method, described herein.

FIG. 27 is a cross-section view of a line contact grip used for the High Speed Tensile Test, described herein.

FIG. 28 is a perspective view of a pair of opposing line contact grips for use in the High Speed Tensile Test, described herein.

FIG. 29 is a graphical illustration of a suitable deformation regimen for the High Speed Tensile Test, described herein.

FIG. 30 is a top view of a strikethrough plate used in the Acquisition Time and Rewet Method, described herein.

FIG. 31 is a bottom view of the strikethrough plate used in the Acquisition Time and Rewet Method, described herein.

FIG. 32A is a cross section view of the strikethrough plate used in the Acquisition Time and Rewet Method described herein, taken along a plane defined by the z-direction and line A-A shown in FIG. 30.

FIG. 32B is a cross section view of the strikethrough plate used in the Acquisition Time and Rewet Method described herein, taken along a plane defined by the z-direction and line B-B shown in FIG. 30.

### DETAILED DESCRIPTION OF THE INVENTION

### Definitions

For purposes herein, the following terms will have the meanings set forth:

“Absorbent article” means a layered product including an absorbent structure and configured to be worn externally about the lower torso and/or crotch region of a human being, and configured to contain and/or absorb bodily exudates which may include urine, menstrual fluid or feces. Examples of absorbent articles include feminine hygiene pads (also known as catamenial pads or sanitary napkins), panty liners, menstrual underwear, incontinence pads, absorbent underwear (configured for, e.g., managing incontinence), diapers and training pants.

“Activated” refers to a process of mechanically deforming a material in order to cause discrete, incremental sections (or activation regions) of the material to be stretched such as, for example, by incrementally stretching the material in at least one direction.

“Bidirectional”—with respect to a layered composite web structure or any layer component thereof, refers to the directions of two axes in an x-y plane, which intersect at a smaller angle ranging from 20 degrees to 90 degrees.

The term “disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

The term “disposed” is used to mean that an element(s) is formed (joined and positioned) in a particular place or position as a unitary structure with other elements or as a separate element joined to another element.

A web, sheet or film material, or a laminate or composite thereof, is considered to be “extensible” for purposes herein if the material has the ability to stretch or elongate, without rupture or breakage, to at least 100% strain, for example, as described below in the Hysteresis Test.

For purposes herein, a web, sheet or film material, or a laminate or composite thereof is “elastic”, “elastomeric”, or “elastically extensible” for purposes herein if the material has the ability to stretch by at least 100% strain without rupture or breakage at a given load, and upon release of the load the material exhibits a percent set of 35% or less. For example, an elastic material that has an initial length of 25.4 mm can stretch to at least 50.8 mm (100% stretch) and, upon removal of the force, retract to a length of 30.5 mm (i.e., have a set of 5.1 mm or 20% set). Stretch, sometimes referred to as strain, percent strain, engineering strain, draw ratio, or elongation, along with recovery and set may each be determined according to the Hysteresis Test described below. Materials that are extensible but not “elastic” are considered “plastically extensible” materials.

“Joined” encompasses configurations whereby an element is directly secured to another element by affixing the element directly to the other element, and configurations whereby an element is indirectly secured to another element by affixing the element to intermediate member(s), which, in turn are affixed to the other element.

“Lateral”—with respect to an absorbent article or a component thereof, refers to a direction parallel to a horizontal line tangent to the front surfaces of the upper portions of wearer's legs proximate the torso, when the article is being worn normally and the wearer has assumed an even, square, normal standing position. A “width” dimension of any component or feature of an absorbent article is measured along the lateral direction. When the absorbent article or component thereof is laid out flat on a horizontal surface, the “lateral” direction corresponds with the lateral direction relative the structure when it is worn, as defined above. With respect to an absorbent article that is opened and laid out flat on a horizontal planar surface, “lateral” refers to a direction perpendicular to the longitudinal direction and parallel to the horizontal planar surface.

The “lateral axis” of an absorbent article or component thereof is a lateral line lying in an x-y plane and equally dividing the length of the article or the component when it is opened and laid out flat on a horizontal surface. A lateral axis is perpendicular to a longitudinal axis.

“Longitudinal”—with respect to an absorbent article or a component thereof, refers to a direction perpendicular to the lateral direction. A “length” dimension of any component or feature of a layered absorbent structure is measured along the longitudinal direction from its forward extent to its rearward extent. When the absorbent article or component thereof is laid out flat on a horizontal surface, the “longitudinal” direction is perpendicular to the lateral direction relative the structure when it is worn, as defined above.

The “longitudinal axis” of an absorbent article or component thereof is a longitudinal line lying in an x-y plane and equally dividing the width of the article, when the article is opened and laid out flat on a horizontal surface. A longitudinal axis is perpendicular to a lateral axis.

“Liquid impermeable”-refers to one or more properties or features of a film, web material or laminate thereof that cause(s) it to resist passage of aqueous liquid therethrough (from one major surface through to the other opposite major surface), under ordinary conditions of use of absorbent articles. A film, web material or laminate thereof may be liquid impermeable, but also vapor permeable (“breathable”).

“Machine direction”—with respect to a process for manufacturing a web material or a laminate or layered arrangement of web materials, refers to the primary direction of conveyance of the materials along the manufacturing line, viewed from above the manufacturing line. It will be understood that the machine direction may change in absolute directional orientation in space at particular locations along the line, if the manufacturing line is so configured. With respect to an individual roller or nip between a pair of rollers, over or in which a web material or combination of web materials is conveyed, laminated or deformed on a manufacturing line, the “machine direction” is ordinarily perpendicular to the axis(es) of the roller(s), and the “cross direction” is ordinarily parallel to the axis(es) of the rollers.

“Permanently mechanically deformed”-means plastically deformed, fractured or broken, or having individual fiber constituents that have been plastically deformed, fractured, broken and/or directionally realigned or reoriented, by application of mechanical force.

“x-y plane,” with reference to an absorbent article or component thereof when laid out flat on a horizontal surface, means any horizontal plane occupied by the horizontal surface or any layer of the article or component.

“z-direction,” with reference to an absorbent article or component thereof when laid out flat on a horizontal surface, is a direction orthogonal to the x-y plane. When the article is being worn by a user (and thus has been urged into a curving configuration), the “z-direction” at any particular point location on the pad refers to a direction normal to the wearer-facing surface of the pad at the particular point location.

The term “wrinkle” refers to a small fold, ridge or crease.

With respect to an absorbent article or component thereof, the terms “front,” “rear,” “forward” and “rearward” and similar relative locational terms relate to features or regions of the pad corresponding to the position they would occupy as ordinarily worn by a user, corresponding with the front (anterior) and rear (posterior) of the wearer/user's body when standing.

With respect to an absorbent article, “wearer-facing” is a relative locational term referring to a feature of a component or structure of the article that when in use lies closer to the wearer than another feature of the component or structure that lies along the same z-direction line. For example, a topsheet has a wearer-facing surface that lies closer to the wearer than the opposite, outward-facing surface of the topsheet.

With respect to an absorbent article, “outward-facing” (sometimes referred to herein as “garment-facing”) is a relative locational term referring to a feature of a component or structure of the article that when in use lies farther from the wearer than another feature of the component or structure that lies along the same z-direction line. For example, a topsheet has an outward-facing surface that lies farther from the wearer than the opposite, wearer-facing surface of the topsheet.

The terms “top,” “bottom,” “upper,” “lower,” “over,” “under,” “beneath,” “superadjacent,” “subjacent,” and similar vertical positional terms, when used herein to refer to layers, components or other features of a wearable absorbent article, are to be interpreted with respect to the article as it would appear when opened and laid out flat on a horizontal surface, with its wearer-facing surface facing upward and outward-facing surface facing downward.

### DESCRIPTION

The present disclosure relates to disposable absorbent articles, and more particularly, to disposable absorbent articles having improved flexibility and conformability. In some configurations, the absorbent article may comprise a topsheet, a backsheet, and an absorbent core structure disposed between the topsheet and the backsheet. The absorbent core structure together with the topsheet and backsheet may be incrementally stretched, causing zones of deformation in the topsheet and/or backsheet and the fracture of the absorbent material into discrete pieces. It was surprisingly found that an absorbent article with an elastic backsheet as described herein can provide the absorbent article with a force threshold at extensions of 10-15 mm that is greater than the peel force of the article during removal from an undergarment and can effectively bring the discrete pieces of absorbent material together to create a relaxed gap of about 280 microns or less. As a result, the absorbent article is able to allow for fluid distribution between the pieces of absorbent material, thus reducing the amount of free fluid present in the channels and/or on the surface of the absorbent article, and resist tearing during removal without losing the desired flexibility and ability to conform closely to the body.

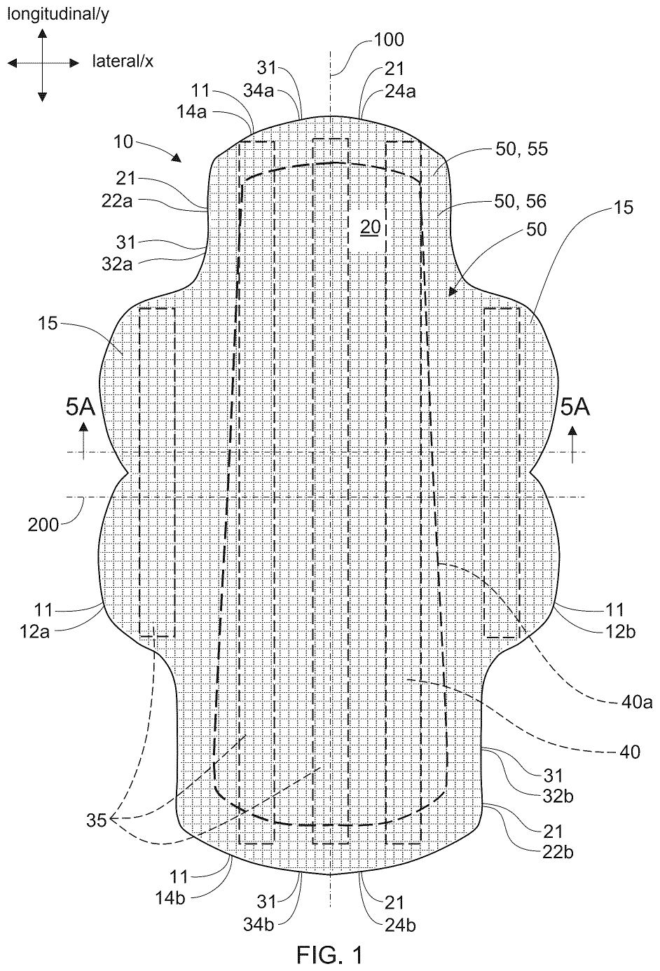

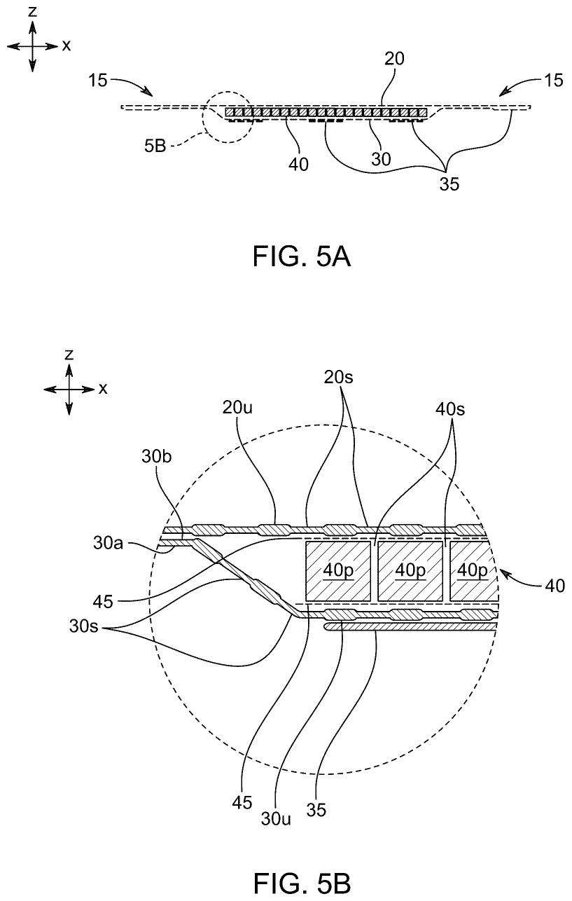

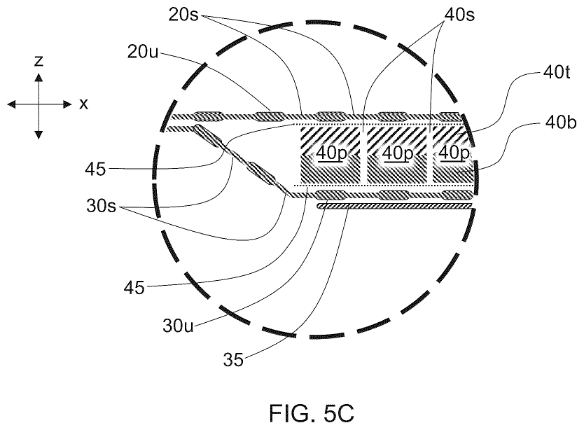

Referring to FIGS. 1 and 5A, an absorbent article 10 (shown here in the form of a feminine hygiene pad) may include a liquid permeable topsheet 20, a liquid impermeable backsheet 30 and an absorbent core structure 40 disposed between the topsheet 20 and the backsheet 30. The backsheet 30 has a garment-facing surface 30a and a wearer-facing surface 30b opposite the garment-facing surface 30a. The absorbent structure 40 has an outer perimeter 40a. In regions outside the outer perimeter 40a, the topsheet and the backsheet may be bonded together in laminate configuration by any suitable mechanism including but not limited to adhesive bonding, thermal bonding, pressure bonding, etc., thereby retaining and holding the absorbent core structure 40 in an enveloped space between the topsheet 20 and the backsheet 30. Article 10 may include opposing wing portions 15 extending laterally outside of perimeter 40a by a comparatively greater width dimension than that of the forwardmost and rearwardmost portions of the pad. The outer surface of the backsheet forming the undersides of the main portion and the wing portions 15 may have one or more deposits of panty fastening adhesive 35 applied thereon. Panty fastening adhesive 35 may be provided to enable the user to adhere the absorbent article to the inside of her underpants in the crotch region thereof, and to wrap the wing portions 15 through and around the inside edges of the leg openings of the underpants and adhere them to the outside/underside of the underpants in the crotch region, providing supplemental holding support and helping to protect the insides of the leg edges of the underpants against soiling.

The panty fastening adhesive 35 may be any adhesive or glue used in the art for such purposes. These adhesives typically are pressure sensitive and remain tacky well below their application temperature. In some configurations, the panty fastening adhesive may be a pressure sensitive hot melt adhesive. Non-limiting examples of panty fastening adhesive can include elastomeric pressure sensitive adhesive PL501K available from Savare Specialty Adhesives or DF6530 available from Henkel Corporation. The basis weight of the panty fastening adhesive applied may be from about 3 gsm to about 22 gsm, or from about 5 gsm to about 20 gsm, or from about 10 gsm to about 18 gsm.

The panty fastening adhesive 35 may be applied onto a garment-facing surface of the absorbent article 10, typically on a garment-facing surface 30a of the backsheet 30 and/or the wings 15, using any one of methods well known in the art for this purpose such as slot coating, spraying and roll printing. One method of applying the panty fastening adhesive to the garment-facing surface of the absorbent article is the direct coating on the backsheet; another method is printing the panty fastening adhesive onto a release paper, which is then pressed onto the garment-facing surface of the absorbent article. Thereby the panty fastening adhesive is transferred from the release paper to the garment-facing surface of the absorbent article. Such a procedure is described in EP 788,338.

Panty fastening adhesive 35 can be applied on at least a portion of the backsheet 30 of the absorbent article 10 in a pattern. In some configurations, the area of the backsheet and/or the constricting layer covered by panty fastening adhesive may be between about 10% and 95%, or from about 15% to about 75%, or from about 20% to about 60%.

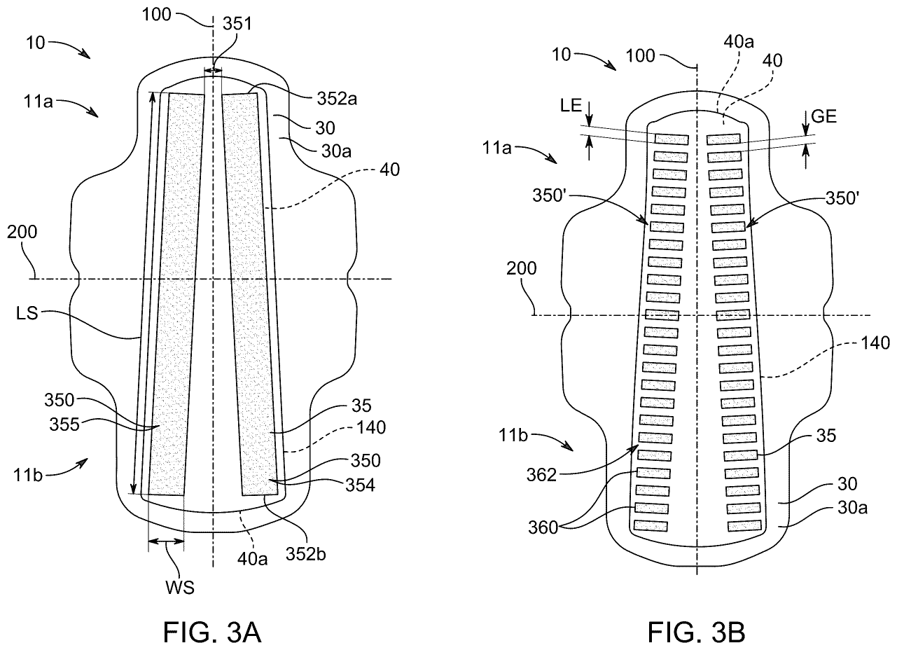

FIG. 3A shows an exemplary pattern of panty fastening adhesive 35. The pattern may comprise one or more stripes 350. In some configurations, the orientation of the stripes 350 may be a direction perpendicular to the direction along which the article 10 is pulled during removal from the undergarment. In other configurations, the orientation of the stripes 350 may be a direction parallel to the direction along which the article 10 is pulled during removal from the undergarment.

In some configurations, the stripes may extend from a first end 352a disposed in a front region 11a of the article to a second end 352b in the rear region 11b of the article. The stripes 350 may have a longitudinal length (LS) of from about 10 mm to about 600 mm, or from about 50 mm to about 400 mm, or from about 100 mm to about 300 mm. The stripes 350 may have a lateral width (WS) of from about 1 mm to about 20 mm, or from about 5 mm to about 18 mm.

In some configurations, the pattern may comprise a first stripe 354 and a second stripe 355. The first stripe 354 may be located on one side of the longitudinal axis 100 of the absorbent article 10 and the second stripe 355 may be located on the other side of the longitudinal axis 100. A gap 351 extending over the longitudinal axis 100 may be provided between the first stripe 354 and the second stripe 355, wherein the gap 351 may have a minimum gap width of from about 5 mm to about 20 mm as measured substantially parallel to the lateral axis 200. In some configurations, the stripes 350 may be disposed from about 1 mm to about 10 mm, or from about 2 mm to about 8 mm, inboard of the longitudinal edge 140 of the absorbent core structure 40.

FIG. 3B shows another exemplary pattern of panty fastening adhesive 35 wherein the stripes 350′ are discontinuous. In some configurations, stripes 350′ may comprise a plurality of adhesive elements 360. Stripes 350′ may comprise a succession of adhesive elements 360 and adhesive free regions 362. Individual adhesive elements 360 may have a longitudinal length (LE) of from about 2 mm to about 10 mm, or from about 5 mm to about 8 mm, and a longitudinal gap size (GE) of from about 1 mm to about 8 mm, or from about 2 mm to about 6 mm. The adhesive elements 360 may be any suitable shape, such as for example rectangles, circles, ovals, geometric figures, stars, and the like.

In some configurations, the panty fastening adhesive 35 may be applied in a discontinuous pattern such as described in U.S. Pat. No. 11,684,523 and/or US 2020/0281782.

When article 10 is packaged, panty fastening adhesive 35 may be covered by one or more sheets of release film or paper (not shown) that covers/shields the adhesive deposits from contact with other surfaces until the user is ready to remove the release film or paper and place the pad in her underpants for wear/use. Any commercially available release paper or film may be used. Suitable examples include BL 30 MG-A SILOX EI/O, BL 30 MG-A SILOX 4 P/O available from Akrosil Corporation, and M&W films available from Gronau in Germany, under the code X-5432.

### Topsheet

Topsheet 20 may be formed of any suitable nonwoven web material that has plastic/non-elastic extensibility suitable for the purposes described herein. Referring back to the figures, the topsheet 20 is positioned adjacent a wearer-facing surface of the absorbent core structure 40 and may be joined thereto and to the backsheet 30 by any suitable attachment or bonding method. The topsheet 20 and the backsheet 30 may be joined directly to each other in the peripheral regions outside the perimeter 40a of the absorbent core structure 40 and may be indirectly joined by directly joining them respectively to wearer-facing and outward-facing surfaces of the absorbent structure.

The article 10 may have any known or otherwise effective topsheet 20, such as one which is compliant, soft feeling, and non-irritating to a wearer's skin. A suitable topsheet material will include a liquid pervious material that is comfortable when in contact with the wearer's skin and permits discharged menstrual fluid to rapidly penetrate through it. A suitable topsheet may be made of any of various materials such as woven or knitted materials, nonwoven web materials, or apertured films.

Nonlimiting examples of nonwoven web materials that may be suitable for use to form the topsheet 20 include fibrous materials made from natural fibers, modified natural fibers, synthetic fibers, or combinations thereof. Some suitable examples are described in U.S. Pat. Nos. 4,950,264; 4,988,344; 4,988,345; 3,978,185; 7,785,690; 7,838,099; 5,792,404; and 5,665,452. Particularly suitable topsheet materials may include a spunbond nonwoven material comprising polyethylene (PE)/polypropylene (PP) bicomponent fibers (PE sheath and PP core).

In some configurations, the topsheet may be a highly extensible nonwoven web comprising staple or continuous multi-component fibers, such as, for example, described in US 2020/0337910A1. A highly extensible nonwoven web may be beneficial to reduce fiber breakage under mechanical processing that may cause a nonwoven web to experience high strain forces, for example during the incremental stretching process. In the absorbent article context, this may have the desirable effect of reducing the amount of broken fibers that stick to the skin of a wearer.

In some configurations, the topsheet may comprise a nonwoven web having an extensibility of between about 300% and about 500%, between about 310% and about 425%, or between about 320% and about 375%, specifically reciting all 1% increments within the specified ranges and all ranges formed therein or thereby, according to the High Speed Tensile Test. A highly extensible nonwoven web with an extensibility within the ranges stated above may be beneficial to reduce fiber breakage under mechanical processing that may cause a nonwoven web to experience high strain forces. Reduced fiber breakage may result in a stronger nonwoven web with reduced lint.

In some configurations, the topsheet 20 may comprise a plurality of apertures. In some configurations, it may be preferable to have a topsheet that does not comprise apertures to help keep the foam pieces from escaping the article during use.

In some configurations, the topsheet 20 may have a basis weight of about 10 gsm (herein, “gsm” means grams/m²) to about 50 gsm, of from about 22 gsm to about 45 gsm, or from about 25 gsm to about 30 gsm, specifically reciting all values within these ranges and any ranges created thereby.

In some configurations, the topsheet 20 may comprise tufts as described in U.S. Pat. Nos. 8,728,049; 7,553,532; 7,172,801; 8,440,286; 7,648,752; and 7,410,683. The topsheet 20 may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535; and US 2015/041640. Another suitable topsheet may be formed from a three-dimensional substrate as detailed in US 2017/0258647. The topsheet may have one or more layers, as described in US 2016/0167334; US 2016/0166443; and US 2017/0258651.

As contemplated herein, component nonwoven web material from which topsheet 20 may be cut may be a nonwoven web material that includes or even consists predominantly (by weight) or entirely of cellulosic plant fibers such as fibers of cotton, flax, hemp, jute or mixtures thereof, that are either naturally hydrophilic or suitably processed so as be rendered hydrophilic (or have increased hydrophilicity), and processed to be suitably soft-feeling against the skin. In other configurations, semisynthetic fibers derived from cellulosic material, such as rayon (for purposes herein, “rayon” includes viscose, lyocell, MODAL (a product of Lenzing AG, Lenzing, Austria) and cuprammonium rayon) may be included as a component of the nonwoven.

The nonwoven web may be formed via any suitable process by which fibers of finite lengths (e.g., staple fibers) may be distributed and accumulated in a controlled fashion onto a forming belt to form a batt having a desired distribution of fibers, to a desired basis weight. Suitable processes may include carding, airlaying and wetlaying. The batt may be processed to consolidate the fibers and entangle them in the z-direction, by processes that may include calendering, needlepunching and hydroentanglement via water jets.

In some configurations, the nonwoven web material may be formed via a carding process. In other configurations, the nonwoven web material may be formed via an airlaying or wetlaying process. In some configurations, the nonwoven web material may be a spunbond web including single-component continuous fibers spun from polymeric resin, or alternatively, bi-component or multi-component fibers, or a blend of single-component fibers spun of differing polymer resins, or any combination thereof. In some configurations, the nonwoven web can be calendar, thermal, ultrasonic, resin, or air through bonded. In some configurations, a web may be formed in a co-forming process in which plant-based fibers of finite lengths are physically blended or mixed with streams of spun fibers of longer but indefinite lengths, spun from polymeric resin, and laid down on a forming belt to form a web as described in, for example, U.S. Pat. Nos. 8,017,534; 4,100,324; US 2003/0200991; U.S. Pat. No. 5,508,102; US 2003/0211802; EP 0 333 228; WO 2009/10938; US 2017/0000695; US 2017/0002486; U.S. Pat. No. 9,944,047; 2017/0022643; and US 2018/0002848.

### Absorbent Core Structure

The absorbent core structure 40 of the present disclosure may be imparted with any suitable shape including, but not limited to, an oval, a discorectangle, a rectangle, an asymmetric shape, and an hourglass. For example, in some configurations, the absorbent core structure 40 may have a contoured shape, e.g., narrower in the intermediate region than in the end regions. As yet another example, the absorbent core structure 40 may comprise a tapered shape having a wider portion in one end region of the pad which tapers to a narrower end region in the other end region of the pad. Where the article is an absorbent pad intended for use by a woman, it may be desired that the rearward end region be wider and the forward end region be narrower. Where the article is an absorbent pad intended for use by a man, it may be desired that the rearward end region be narrower and the forward end region be wider. The absorbent core structure 40 may comprise varying stiffnesses in the longitudinal and lateral directions.

The configuration and construction of the absorbent core structure 40 may vary (e.g., the absorbent core structure 40 may have varying caliper zones, a hydrophilic gradient, a superabsorbent gradient, or lower average density and lower average basis weight acquisition zones). Further, the size and absorbent capacity of the absorbent core structure 40 may also be varied to accommodate a variety of wearers. However, the total absorbent capacity of the absorbent core structure 40 should be compatible with the design loading and the intended use of the absorbent article. In some configurations, the absorbent core structure may have an absorbent capacity of from about 4.0 grams to about 10.0 grams of water per gram of absorbent material.

In some forms, the absorbent core structure 40 may include a plurality of multi-functional layers. For example, the absorbent core structure 40 may include a core wrap (not shown) useful for enveloping other layers. The core wrap may be formed by one or two sections of nonwoven material, substrates, laminates, films, or other materials. In some configurations, the core wrap may be formed of a single material, or laminate, wrapped at least partially around itself.

The absorbent core structure 40 may include one or more adhesives, for example, to help immobilize absorbent gelling material/superabsorbent polymer (SAP) or other absorbent materials included within the absorbent structure.

Absorbent structures comprising relatively high amounts of SAP with various absorbent structure/core designs are disclosed in U.S. Pat. No. 5,599,335; EP 1,447,066; WO 95/11652; US 2008/0312622A1 to Hundorf et al.; and WO 2012/052172. These may be used to configure the superabsorbent layers.

Additions to the absorbent core structure of the present disclosure are envisioned. In particular, potential additions to the current multi-laminate absorbent structure are described in U.S. Pat. Nos. 4,610,678; 4,673,402; 4,888,231; and 4,834,735. The absorbent core structure may further comprise additional layers that mimic the dual core system containing an acquisition/distribution structure of chemically stiffened fibers positioned over an absorbent structure as detailed in U.S. Pat. No. 5,234,423; and in U.S. Pat. No. 5,147,345. These are useful to the extent they do not negate or conflict with the effects of the below described laminates of the absorbent structure.

Some examples of suitable absorbent core structures 40 that can be used in the absorbent article of the present disclosure are described in US 2018/0098893 and US 2018/0098891. Examples of possible suitable configurations are further described and depicted in U.S. application Ser. No. 16/831,851.

The absorbent core structure 40 may be formed of or include a layer of absorbent open-celled foam material. In some examples, the foam material may include at least first and second sublayers (e.g., 40t, 40b, see FIG. 5C) of absorbent open-celled foam material, the sublayers being in direct face-to-face contact with each other. In such examples, the wearer-facing sublayer may be a relatively larger-celled foam material, and the outward-facing sublayer may be a relatively smaller-celled foam material, for purposes explained in more detail below.

The open-celled foam material may be a foam material that is manufactured via polymerization of the continuous oily monomer phase of a water-in-oil high internal phase emulsion (“HIPE”). A HIPE for purposes herein is a two-phase water-in-oil emulsion in which the water-to-oil ratio is greater than about 2.85:1, i.e., about 74 percent aqueous/dispersed phase (by volume). Due to this relatively high ratio of aqueous phase to oil phase, upon emulsification sufficient to result in a foam of the type contemplated herein (i.e., average cell size 1 to 300 micrometers), the dispersed aqueous phase will have the form of droplets forced into polyhedral forms as a result of close crowding, separated by thin film walls formed of the oil/continuous phase. An open-celled foam material as described herein may be preferred for purposes herein, because it may be manufactured to be relatively brittle under tension, such that it can be caused to fracture neatly along orderly lines to form approximately or substantially uniformly sized and shaped pieces, in the deformation process described herein.

The oil phase of a HIPE is continuous and includes monomers to be polymerized, and an emulsifier to help create and stabilize the HIPE. The oil phase may also include one or more photoinitiators. The monomer component may be included in an amount of from about 80% to about 99%, by weight of the oil phase. The emulsifier component, which is soluble in the oil phase and suitable for forming a stable water-in-oil emulsion may be included in the oil phase in an amount of from about 1% to about 20% by weight of the oil phase. The emulsion may be formed at an emulsification temperature of from about 20° C. to about 130° C.

In general, the monomers may be included in an amount of about 20% to about 97% by weight of the oil phase and may include at least one substantially water-insoluble monofunctional alkyl acrylate or alkyl methacrylate. For example, monomers of this type may include C4-C18 alkyl acrylates and C2-C18 methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40%, by weight of the oil phase, a polyfunctional crosslinking comonomer. This crosslinking comonomer, or crosslinker, is added to confer strength and resilience to the resulting HIPE foam. Examples of crosslinking monomers of this type comprise monomers containing two or more activated acrylate, methacrylate groups, or combinations thereof. Nonlimiting examples of this group include 1,6-hexanedioldiacrylate, 1,4-butanedioldimethacrylate, trimethylolpropane triacrylate, trimethylolpropane trimethacrylate, 1,1 2-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like.

Any third substantially water-insoluble comonomer may be added to the oil phase in weight percentages of from about 0% to about 15% by weight of the oil phase, to modify properties of the HIPE foams. In certain cases, “toughening” monomers may be desired to impart toughness to the resulting HIPE foam. These include monomers such as styrene, vinyl chloride, vinylidene chloride, isoprene, and chloroprene. Without intending to be bound by theory, it is believed that such monomers aid in stabilizing the HIPE during polymerization (also known as “curing”) to provide a more homogeneous and better-formed HIPE foam which results in greater toughness, tensile strength, abrasion resistance, and the like. Monomers may also be added to confer flame retardancy, as disclosed, for example, in U.S. Pat. No. 6,160,028. Monomers may be added to impart color (for example vinyl ferrocene); to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam. In some cases, these additional monomers may slow the overall process of conversion of HIPE to HIPE foam, the tradeoff being necessary if the desired property is to be conferred. Thus, such monomers can also be used to slow down the polymerization rate of a HIPE. Examples of monomers of this type comprise styrene and vinyl chloride.

The oil phase may further include an emulsifier to facilitate emulsification and stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate, polyglycerol monoisostearate, and polyglycerol monomyristate; (b) polyglycerol monoesters of -branched C16-C24 fatty acids, linear unsaturated C16-C22 fatty acids, or linear saturated C12-C14 fatty acids, such as diglycerol monooleate (for example diglycerol monoesters of C18: 1 fatty acids), diglycerol monomyristate, diglycerol monoisostearate, and diglycerol monoesters; (c) diglycerol monoaliphatic ethers of -branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate, which is formed from an alkyl succinate, glycerol, and triglycerol.

Such emulsifiers, and combinations thereof, may be added to the oil phase so that they constitute about 1% to about 20% of the weight of the oil phase. In some configurations, coemulsifiers may also be used to provide additional control of cell size, cell size distribution, and emulsion stability, particularly at higher temperatures. Examples of coemulsifiers include phosphatidyl cholines and phosphatidyl choline-containing compositions, aliphatic betaines, long chain C12-C22 dialiphatic quaternary ammonium salts, short chain C1-C₄ dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts. In some configurations, ditallow dimethyl ammonium methyl sulfate may be used as a coemulsifier.

Any photoinitiators included may be included at between about 0.05% and about 10%, by weight of the oil phase. Lower amounts of photoinitiator allow light to better penetrate the HIPE foam, which can provide for polymerization deeper into the HIPE foam. However, if polymerization is performed in an oxygen-containing environment, it may be desired that there be enough photoinitiator present to initiate the polymerization and overcome oxygen inhibition. Photoinitiators can respond rapidly and efficiently to a light source with the production of radicals, cations, and other species that are capable of initiating a polymerization reaction. Photoinitiators selected for use in forming foams within contemplation of the present disclosure may absorb UV light at wavelengths of about 200 nanometers (nm) to about 800 nm, or about 250 nm to about 450 nm. If the photoinitiator is in the oil phase, suitable types of oil-soluble photoinitiators include benzyl ketals, α-hydroxyalkyl phenones, α-amino alkyl phenones, and acylphospine oxides. Examples of photoinitiators include 2,4,6-[trimethylbenzoyldiphosphine]oxide in combination with 2-hydroxy-2-methyl-1-phenylpropan-1-one (50:50 blend of the two is sold by Ciba Speciality Chemicals, Ludwigshafen, Germany as DAROCUR® 4265); benzyl dimethyl ketal (sold by Ciba Geigy as IRGACURE 651); α-,α-dimethoxy-α-hydroxy acetophenone (sold by Ciba Speciality Chemicals as DAROCUR® 1173); 2-methyl-1-[4-(methyl thio)phenyl]-2-morpholino-propan-1-one (sold by Ciba Speciality Chemicals as IRGACURE® 907); 1-hydroxycyclohexyl-phenyl ketone (sold by Ciba Speciality Chemicals as IRGACURE® 184); bis(2,4,6-trimethylbenzoyl)-phenylphosphineoxide (sold by Ciba Speciality Chemicals as IRGACURE 819); diethoxyacetophenone, and 4-(2-hydroxyethoxy)phenyl-(2-hydroxy-2-methylpropyl) ketone (sold by Ciba Speciality Chemicals as IRGACURE® 2959); and Oligo [2-hydroxy-2-methyl-1-[4-(1-methylvinyl)phenyl]propanone] (sold by Lamberti spa, Gallarate, Italy as ESACURE® KIP EM).

The dispersed aqueous phase of a HIPE includes predominately water, and may also include one or more components, such as initiator, photoinitiator, or electrolyte, wherein in certain examples, the one or more components are at least partially water soluble.

One component included in the aqueous phase may be a water-soluble electrolyte. The water phase may contain from about 0.2% to about 40%, by weight of the aqueous phase of a water-soluble electrolyte. The electrolyte minimizes the tendency of monomers, comonomers, and crosslinkers that are primarily oil soluble to also dissolve in the aqueous phase. Examples of electrolytes include chlorides or sulfates of alkaline earth metals such as calcium or magnesium and chlorides or sulfates of alkali earth metals such as sodium. Such electrolyte can include a buffering agent for the control of pH during the polymerization, including such inorganic counterions as phosphate, borate, and carbonate, and mixtures thereof.

Another component that may be included in the aqueous phase is a water-soluble free-radical initiator. The initiator can be present at up to about 20 mole percent based on the total moles of polymerizable monomers present in the oil phase. In some configurations, the initiator may be included in an amount of from about 0.001 to about 10 mole percent based on the total moles of polymerizable monomers in the oil phase. Suitable initiators include ammonium persulfate, sodium persulfate, potassium persulfate, 2,2′-azobis(N,N′-dimethyleneisobutyramidine) dihydrochloride, azo initiators, redox couples like persulfate-bisulfate, persulfate-ascorbic acid, and other suitable redox initiators. In some configurations, to reduce the potential for premature polymerization which may clog the emulsification system, addition of the initiator to the monomer phase may be performed near the end of the emulsification step, or shortly afterward.

Photoinitiator, if included in the aqueous phase, may be at least partially water soluble, and may constitute between about 0.05% and about 10%, by weight of the oil phase. Lower amounts of photoinitiator allow light to better penetrate the HIPE foam, which can provide for polymerization deeper into the HIPE foam. However, if polymerization is done in an oxygen-containing environment, there should be enough photoinitiator to initiate the polymerization and overcome oxygen inhibition. Photoinitiators can respond rapidly and efficiently to a light source with the production of radicals, cations, and other species that are capable of initiating a polymerization reaction. Photoinitiators selected for use to form foams within contemplation of the present disclosure may absorb UV light at wavelengths of from about 200 nanometers (nm) to about 800 nm, or from about 200 nm to about 350 nm, or from about 350 nm to about 450 nm. If a photoinitiator is to be included in the aqueous phase, suitable types of water-soluble photoinitiators may include benzophenones, benzils, and thioxanthones. Examples of photoinitiators include 2,2′-Azobis [2-(2-imidazolin-2-yl) propane]dihydrochloride; 2,2′-Azobis [2-(2-imidazolin-2-yl) propane]disulfate dehydrate; 2,2′-Azobis(1-imino-1-pyrrolidino-2-ethylpropane) dihydrochloride; 2,2′-Azobis [2-methyl-N-(2-hydroxyethyl) propionamide]; 2,2′-Azobis(2-methylpropionamidine) dihydrochloride; 2,2′-dicarboxymethoxydibenzalacetone, 4,4′-dicarboxymethoxydibenzalacetone, 4,4′-dicarboxymethoxydibenzalcyclohexanone, 4-dimethylamino-4′-carboxymethoxydibenzalacetone; and 4,4′-disulphoxymethoxydibenzalacetone. Other suitable photoinitiators that can be used are listed in U.S. Pat. No. 4,824,765.

In addition to the previously described components, other components may be included in either the aqueous or oil phase of a HIPE. Examples include antioxidants, for example hindered phenolics, hindered amine light stabilizers; plasticizers, for example dioctyl phthalate, dinonyl sebacate; flame retardants, for example halogenated hydrocarbons, phosphates, borates, inorganic salts such as antimony trioxide or ammonium phosphate or magnesium hydroxide; dyes and pigments; fluorescers; filler particles, for example starch, titanium dioxide, carbon black, or calcium carbonate; fibers; chain transfer agents; odor absorbers, for example activated carbon particulates; dissolved polymers; dissolved oligomers; and the like.

HIPE foam is produced from the polymerization of the monomers comprising the continuous oil phase of a HIPE. In some configurations, a HIPE foam layer may be manufactured so as to have one or more sublayers (e.g., 40t, 40b, see FIG. 5C), and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). For example, for a HIPE foam if the difference relates to pore size, the average pore size in the respective sublayers may differ by at least about 20%, or by at least about 35%, or by at least about 50%. In another configuration, if the differences in the sublayers of a HIPE foam layer relate to density, the densities of the layers may differ by at least about 20%, or by at least about 35%, or by at least about 50%. For instance, if one layer of a HIPE foam has a density of 0.020 g/cm³, another layer may have a density of at least about 0.024 g/cm³ or less than about 0.016 g/cm³, in some configurations at least about 0.027 g/cm³ or less than about 0.013 g/cm³, and in still other configurations at least about 0.030 g/cm³ or less than about 0.010 g/cm³. If the differences between the layers are related to the chemical composition of the HIPE or HIPE foam, the differences may reflect a relative amount difference in at least one monomer component, for example by at least about 20%, in some configurations by at least about 35%, and in still further configurations by at least about 50%. For instance, if one sublayer of a HIPE or HIPE foam is composed of about 10% styrene in its formulation, another sublayer of the HIPE or HIPE foam may be composed of at least about 12%, and in certain examples of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent structure of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from about 1 to about 200 μm, and in certain configurations may be less than about 100 μm. HIPE foam layers of the present disclosure having two major parallel surfaces may be from about 0.5 to about 10 mm thick, and in certain configurations from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed fluid communication with adjoining cells. The cells in such substantially open-celled HIPE foam structures have intercellular openings or windows that are large enough to permit ready fluid transfer from one cell to another within the HIPE foam structure. For purpose of the present disclosure, a HIPE foam is considered “open-celled” if at least about 80% of the cells in the HIPE foam that are at least 1 μm in size are in fluid communication with at least one adjoining cell.

In addition to being open-celled, in certain examples HIPE foams are adapted to be sufficiently hydrophilic to permit the HIPE foam to absorb aqueous fluids. In some examples the internal surfaces of a HIPE foam may be rendered hydrophilic by residual hydrophilizing surfactants or salts left in the HIPE foam following polymerization, or by selected post-polymerization HIPE foam treatment procedures such as those as described in references cited herein.

In some configurations, for example when it is used to form an absorbent structure of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In some configurations, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In some configurations, the Tg of this region will be less than about 40° C. for foams used at about ambient temperature conditions; in certain other configurations Tg will be less than about 30° C. For HIPE foams used in applications wherein the use temperature is higher or lower than ambient temperature, the Tg of the continuous region may be no more than 10° C. greater than the use temperature, in certain configurations the same as use temperature, and in further examples about 10° C. less than use temperature wherein flexibility is desired. Accordingly, monomers are selected as much as possible that provide corresponding polymers having lower Tg's.

HIPE foams useful for forming absorbent structures and/or sublayers within contemplation of the present disclosure, and materials and methods for their manufacture, also include but are not necessarily limited to those foams and methods described in U.S. Pat. Nos. 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; U.S. Pat. Nos. 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; U.S. Pat. Nos. 5,500,451; 5,387,207; 5,352,711; U.S. Pat. Nos. 5,397,316; 5,331,015; 5,292,777; 5,268,224; 5,260,345; 5,250,576; 5,149,720; 5,147,345; and US 2005/0197414; US 2005/0197415; US 2011/0160326; US 2011/0159135; US 2011/0159206; US 2011/0160321; and US 2011/0160689, which are incorporated herein by reference to the extent not inconsistent herewith.

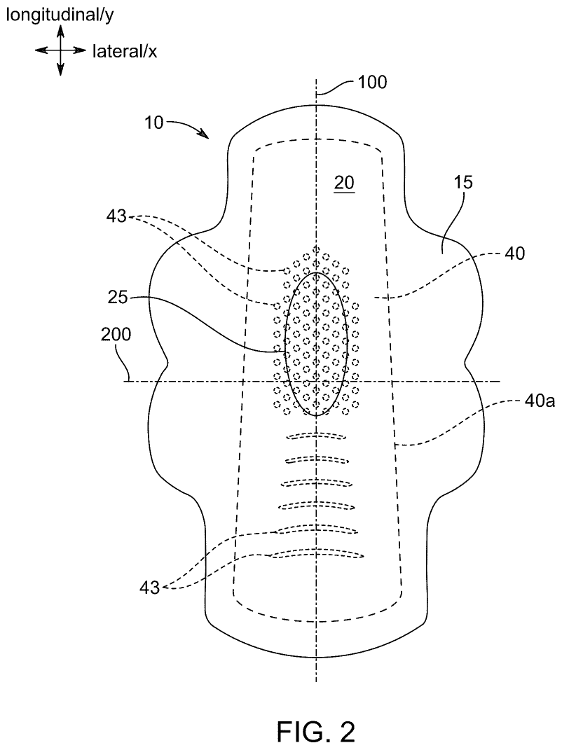

As reflected in FIG. 2, an absorbent core structure 40 formed of HIPE foam may include one or more patterns of apertures 43, including at least a first pattern disposed within an expected discharge location overlying the intersection of the longitudinal and lateral axes 100, 200 of the absorbent article. Apertures 43 may be punched, cut or otherwise formed through the entire z-direction depth of the HIPE foam absorbent core structure, or only through a wearer-facing layer or partially into the wearer-facing portion thereof. When a HIPE foam absorbent core structure is disposed in direct contact with a topsheet as described herein, with no intervening acquisition layer formed of another material, apertures 43 may serve as a group of reservoirs to receive, temporarily hold, and aid in distributing rapid discharges of relatively small quantities of menstrual fluid, until the HIPE foam has sufficient time to distribute and absorb the fluid via capillary action. Additionally, such apertures help decrease bending stiffness of the absorbent core structure, which may help increase comfort of the absorbent article for the wearer. A pattern of apertures having an average radius or other largest dimension of about 1.0 mm to about 4.0 mm, or about 1.5 mm to about 3.5 mm may be included, within, for example, the area occupied by the bonding region 25. The pattern may include apertures at a numerical density of about 3.0 to about 9.0 apertures per cm², and or about 4.0 to 8.0 apertures per cm². In selecting the appropriate average size, numerical density, and surface area occupied by the pattern of apertures, the manufacturer may wish to balance the volume of the “reservoirs” desired with the need to retain absorbent material in locations proximate to and about the expected discharge location. Additional details concerning configurations of such apertures in combination with examples of suitable absorbent core structures may be found in U.S. Pat. No. 8,211,078.

The absorbent core structure 40 formed of HIPE foam should be imparted with sufficient capillary work potential in absorption mode (CWPA) (described below) to have capability to effectively draw discharged fluid from a topsheet over a time of use/wear of the pad during menstruation that is normal and expected for feminine hygiene pads, for example, from 4 to 8 hours. For a feminine hygiene pad with a HIPE foam absorbent core structure as contemplated herein, it may be desired that the HIPE foam layer have a caliper in the majority of its wearer-facing surface area (prior to wetting) of about 1 mm to about 5 mm, or about 1.5 mm to about 3.5 mm, or about 2.0 mm to about 3.0 mm. (The caliper of a HIPE foam layer may be measured visually, with assistance of magnification/microscopy and/or photography or any other facilitating techniques and equipment, to any extent deemed useful.) Where the absorbent core structure 40 includes two sublayers as described herein, it may be desired that an upper sublayer 40t have a caliper (prior to wetting) of about 0.64 mm to about 3.2 mm, or about 0.96 mm to about 2.24 mm, or about 1.28 mm to about 1.92 mm; and it may be desired that a lower sublayer 40b have a caliper (prior to wetting) of about 0.16 mm to about 0.80 mm, or about 0.24 mm to about 0.56 mm, or about 0.32 mm to about 0.48 mm.

In some configurations, the absorbent core structure 40 may consist of or include a heterogeneous layer consisting of an absorbent foam material (such as a HIPE foam material, as described above) with structure that has been polymerized and thereby formed about, among and/or within the matrix of fibers of a nonwoven web material. Examples of such heterogeneous layers are depicted and described in US2017/0119587; US2017/0119596; US2017/0119597; US2017/0119588; US2017/0119593; US2017/0119594; US2017/0119595; and US2017/0199598.

It will be appreciated that the absorbent structure described herein may be adapted not only for use as a feminine hygiene pad, but also an incontinence pad or absorbent insert for use within underwear, or even as a structural subcomponent of disposable menstrual underwear or disposable adult incontinence underwear adapted for use/wear by men or women.

### Absorbency Properties and Interface Between Topsheet and Absorbent Structure

The affinity and absorbency of an absorbent/hydrophilic structure for an aqueous fluid may be characterized in part by its capillary absorption pressure. Capillary absorption pressure (CAP) may be measured according to steps in the Capillary Work Potential measurement method set forth below. It is a value that reflects the magnitude of the tendency of the structure to draw in aqueous fluid. It will be appreciated that a plot of the CAP of an absorbent structure vs. saturation level will have an initial maximum value (at the outset of absorption of fluid) and decrease as the structure draws in fluid and approaches its full absorption capacity, i.e., full saturation.

The resistance to desorption, or the tendency of an absorbent/hydrophilic structure to retain absorbed fluid, may be characterized in part by its capillary desorption pressure (CDP). CDP, which also may be measured according to steps in the Capillary Work Potential measurement method set forth below, is a value that reflects the magnitude of pressure (or pressure differential) required to drive (or draw) out aqueous fluid absorbed and held in the structure. It will be appreciated that a plot of the CDP of a structure vs. saturation level will have an initial minimum value (prior to exit of any fluid from the structure), and increase as the fluid leaves it.

CAP and CDP of a given structure are a function of the extent of hydrophilicity of the solid surfaces within the structure, the average size of the interstitial spaces or voids, cells or pores within the structure among/between the solid surfaces, and the number of the interstitial spaces, cells or pores within the structure per unit volume of the structure.

In order for a layered topsheet/absorbent structure combination to be able to effectively move discharged fluid from the top surface of the topsheet in a z-direction direction away from the wearer, in addition to other conditions described herein, the CAP of the absorbent core structure must be greater than the CDP of the topsheet, at a selected level of absorbed fluid content of the topsheet, preferably a relatively low level. In order for a layered topsheet/absorbent core structure combination to be able to move discharged fluid in a z-direction direction away from the wearer with acceptable rapidity, i.e., such that the topsheet does not have time to excessively wick and thereby distribute (i.e., spread) discharged fluid along x-y planar directions (creating an undesirably large stain on the topsheet) and does not feel overly wet to the wearer shortly following a discharge of fluid thereon, the capillary absorption pressure of the absorbent core structure at, e.g., a 20 percent saturation should be greater than the capillary desorption pressure of the topsheet at the same saturation, where percent saturation is the percent of total pore volume of the material that is occupied by the fluid, and the test fluid is saline solution as specified in the Capillary Work Potential measurement method set forth below.

The total absorbency of a given material structure may be further characterized by its capillary work potential in absorption mode (CWPA) and drainage or desorption mode (CWPD), as measured using the Capillary Work Potential measurement method set forth below. CWPA is a measure of the work that an absorbent material will perform in drawing in a quantity of aqueous fluid under conditions of the described method. CWPD is a measure of the work necessary to expel or draw away aqueous fluid absorbed and held by a structure under conditions of the described method. For a given structure that is hydrophilic and absorbent of aqueous fluid, the CWPD will be greater than the CWPA because the properties of an absorbent structure (hydrophilicity; cell/pore size and volume) cause it to tend to retain fluid. CWPA and CWPD of a given structure are affected by the features and properties that affect CAP and CDP, and also by the total volume of the interstitial spaces or voids, cells or pores within the structure within the structure. Thus, it will be appreciated that CWPA and CWPD of a structure are in part affected by the total volume (i.e., size) of the structure.

In order to ensure that the absorbent core structure 40 will drain the topsheet 20 of fluid absorbed by the topsheet sufficiently for the two to provide a satisfactory absorbent article, the absorbent core structure 40 should have a CWPA that is greater than the CWPD of the topsheet. If this condition is not satisfied, the absorbent core structure may not sufficiently drain fluid from the topsheet to both (1) ensure that the topsheet will not retain an unacceptably wet feeling following a discharge; and (2) ensure that the topsheet is kept drained and has capacity to accept successive discharges of fluid over a reasonable time of use of the article 10.

It has been learned that an absorbent structure formed of HIPE foam as described herein may be manufactured to have a capillary absorption pressure great enough to draw fluid from an absorbent cotton topsheet with acceptable rapidity over repeated discharges, i.e., over a reasonable time of use of the absorbent article.

In examples in which the topsheet is formed in part or in whole of web material including hydrophilic fibers, the topsheet material may tend to retain fluid on its wearer-facing and outward-facing surfaces, and within the interstitial spaces between and along the surfaces of the fibers within the web material, unless the underlying material has absorption capacity and absorption pressure greater than the desorption pressure of the topsheet, as described above; and there is sufficient direct contact maintained between the topsheet and the underlying absorbent structure to enable the fluid to move from fiber surfaces within the topsheet structure, directly to surfaces of material within the underlying absorbent structure, such that the underlying absorbent structure may draw the fluid from the topsheet. Prior to the time it is fully saturated, an absorbent material will tend not to release absorbed fluid unless an adjacent material with greater affinity for the fluid is in sufficient direct contact. Accordingly, it is important to provide structure sufficient to maintain sufficient contact, without obstructing fluid movement. No intervening layer or structure of material, or at least no intervening layer or structure of material less absorbent than the topsheet or more absorbent than the absorbent core structure, should be interposed between the material of the topsheet 20 and the material of the absorbent core structure 40, at least within the bonding region 25, more preferably over a majority of the wearer-facing surface area of the absorbent core structure 40, and even more preferably over the entirety of the wearer-facing surface area of the absorbent core structure 40—unlike systems provided in many current feminine hygiene pads, which include a distinct fluid acquisition/distribution material layer between the topsheet and the absorbent materials of the absorbent structure.

Sufficient direct contact between the topsheet 20 and the absorbent core structure 40 may be effected by deposit(s) of adhesive between the topsheet 20 and the absorbent structure 40, adhesively bonding them in close z-direction proximity. The adhesive may be applied in a pattern or arrangement of adhesive deposits interspersed with areas in which no adhesive is present (unbonded areas), such that the adhesive holds the two layers in close z-direction proximity, while areas remain in which no adhesive is present to obstruct z-direction fluid movement between the layers.

Referring to FIG. 2, to ensure that the topsheet 20 and absorbent core structure 40 are held in sufficiently close z-direction proximity at least in the area of the topsheet 20 expected to receive a discharge of fluid, it may be desired to dispose a bonding region 25 on the absorbent article at a location that includes the intersection of the longitudinal and lateral axes 100, 200. The bonding region 25 should be of sufficient size to be reliably present beneath the expected discharge location when the article is in use, with reasonably minor variations of placement by the wearer within the underpants; accordingly, it may be desired that the bonding region have an area of at least about 15 cm², or at least about 30 cm². Even more preferably, it may be desired that the bonding region 25 have an area that is at least half of the total wearer-facing surface area of the absorbent core structure 40 (within its perimeter 40a). (Note: FIG. 2 is not presented herein as actual size or scale depiction.)

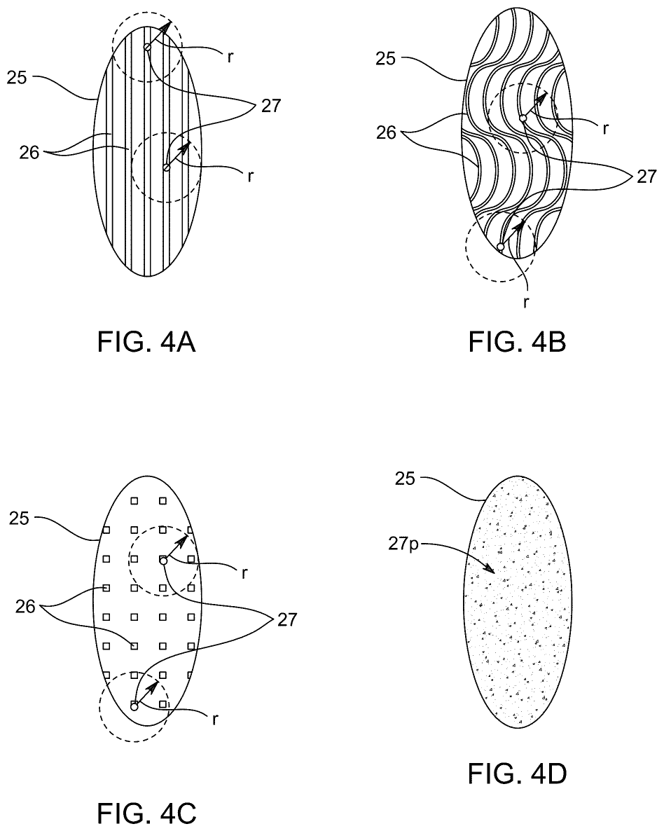

Referring to FIGS. 2 and 4A-4C, to ensure that the topsheet 20 and the absorbent core structure 40 remain in sufficient z-direction proximity during use, it may be desired that, within any identifiable first point location of bonding 27 within the bonding region 25, at which the topsheet is bonded to the absorbent core structure, there is a second identifiable point location at which the topsheet is bonded to the absorbent core structure, within an about 10 mm radius, more preferably within an about 6 mm radius, about 5 mm radius, about 4 mm radius, and even more preferably with an about 3 mm radius r of the first point location. Referring to FIGS. 4A-4C, depicting three non-limiting configurations, it can be seen that a variety of patterns or arrangements of bonds (via adhesive deposits 26 or other bonding mechanisms) may be employed to impart this feature. Within radius r of each identifiable point location of bonding 27, there are a number of additional point locations where bonding between the topsheet and the absorbent core structure is present in the examples depicted.

It will be appreciated that a continuous film- or coating-like deposit of adhesive may be applied to bond the topsheet and the absorbent core structure within the entirety of bonded region 25, but that such a continuous deposit of adhesive could form an occlusive barrier that would obstruct the movement of fluid from the topsheet and into the absorbent structure. Accordingly, it is preferable that, in examples in which the bonding mechanism is deposits of adhesive, the deposits are disposed in a pattern or arrangement that is discontinuous or intermittent such that it creates bonded areas interspersed with unbonded areas between the topsheet and the absorbent core structure, as suggested in FIGS. 4A-4C.

In another configuration suggested in FIG. 4D, a dense arrangement of relatively small point bond locations may be effected by spraying suitable adhesive onto one or both of the outward-facing surface of the topsheet 20 and the wearer-facing surface of the absorbent core structure in contact with the outward-facing surface of the topsheet. If the adhesive suitably adapted (e.g., its viscosity), the spray nozzle is suitably configured, and the rate of spray (liquid volume or weight of adhesive sprayed/time/surface area of spray coverage) is suitably regulated, such that discrete spray droplets strike and adhere to the surface in discrete locations, to an extent suitably limited such that a continuous deposit or continuous film is not formed, the sprayed adhesive can form a dense random pattern 27p of discrete point bonding locations that falls within the description in the preceding paragraph hereinabove, but does not result in a continuous film of deposited adhesive that occludes pores of the underlying absorbent structure or obstruct the movement of fluid from the topsheet to the underlying absorbent structure.

Additionally, when the absorbent core structure is formed of an open-celled foam (such as a HIPE foam contemplated herein) it may be desired that the adhesive selected not effect adhesion to the absorbent structure via chemical, dispersive or diffusive adhesion with the foam layer at the adhesive deposit locations, but rather, that it effect adhesion to the foam layer mechanically, by flowing to a limited extent into the cells, at least partially assuming the shapes thereof, and solidifying in such position to form mechanical interlocks with the cell structures, which enable the adhesive to hold the topsheet and/or backsheet to the absorbent structure. Such an adhesive may be preferred so as not to alter the molecular structure or composition of the foam material, potentially negatively affecting its fluid absorption properties or mechanical strength. In some configurations, a portion of the adhesive may penetrate into the wearer-facing surface of the foam material and into the garment-facing surface of the topsheet and/or a portion of the adhesive may penetrate into the garment-facing surface of the foam material and into the wearer-facing surface of the backsheet to bond the foam material to the topsheet and/or backsheet. One suitable example may be an adhesive of the designation H2031-C5X, a product of Bostik, a division or subsidiary of the Arkema Group, Columbes, France.

### Backsheet

In some configurations, the absorbent article 10 may comprise an elastic backsheet. The backsheet may comprise an elastic layer which is elastically extensible. In some configurations, the backsheet may further comprise a constricting layer which is plastically deformable. When present, the constricting layer can have a yield point at a force that is significantly higher than the elastic layer at the same extension (corresponding to the point of extension of yield of the constricting layer). Particularly suitable constricting layers can plastically deform without breaking throughout an extension range that corresponds to the extension range that the backsheet undergoes during incremental stretching in the activation regions. Because the constricting layer can retain its structural integrity, it can provide the backsheet with a force wall throughout the extension range (i.e., 0% extension to 100% extension) of the backsheet.

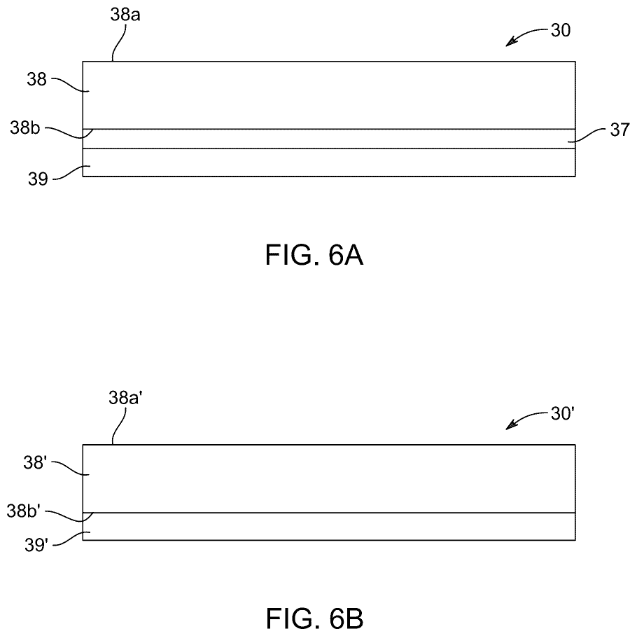

FIG. 6A illustrates an exemplary configuration of a backsheet 30 according to the present disclosure. According to this configuration, backsheet 30 may be a laminate comprising an elastic layer 38 attached to a constricting layer 39. For example, adhesive 37 may be disposed between the elastic layer 38 and the constricting layer 39. While adhesive 37 appears as a continuous layer in FIG. 6A, the adhesive may be applied as a continuous pattern or in a discontinuous pattern (such as a pattern of vertical stripes, horizontal lines, spirals, omega dots, or spots (i.e., filaments of adhesive applied using meltblown applicators). Accordingly, the bonding can be the full width of the backsheet 30 or a partial width of the laminate (e.g., intermittent or zone bonding). Further, alternative attachment mechanisms may include thermal bonding, ultrasonic bonding, calendar bonding, pressure bonding, extrusion bonding, or any other suitable attachment mechanism or combination of these attachment mechanisms. According to other configurations (as depicted in FIG. 6B), a backsheet 30′ may be a unitary structure comprising a constricting layer 39′ co-extruded with elastic layer 38′ (multi-layer elastic film). In some configurations, a backsheet 30′ may comprise a co-extruded elastic film that has a constricting layer on one surface, while in other configurations the constricting layer is co-extruded on both surfaces of an elastic layer. Although the following description will refer to the specific reference numbers in FIG. 6A, the prime versions of those numbers relating to the unitary structure of FIG. 6B are also intended to be considered by the reader.

The backsheet 30 may comprise a single constricting layer or multiple constricting layers. While the layers 38 and 39 appear to overlie each other completely, this need not be the case in all embodiments. For instance, the constricting layer 39 may extend beyond elastic layer 38, alternatively the constricting layer 39 may not extend to the limits of elastic layer 38.

Elastic layer 38 may be made of a single layer or multiple layer material that is elastically extensible. The elastic layer 38 may have a thickness, prior to activation, of about 10 μm to about 100 μm, or from about 20 μm to about 60 μm, or from about 30 μm to about 50 μm. The elastic layer 38 may have a basis weight of from about 5 gsm to about 60 gsm, or from about 10 gsm to about 30 gsm.

The elastically extensible material may comprise thermoplastic elastomers selected from the group consisting of styrenic block copolymers, poly-esters, polyurethanes, polyether amides, and combinations thereof. Suitable styrenic block copolymers may be diblock, triblock, tetrablock, or other multi-block copolymers having at least one styrenic block. Exemplary styrenic block copolymers include styrene-butadiene-styrene, styrene-isoprene-styrene, styrene-ethylene/butylenes-styrene, styrene-ethylene/propylene-styrene, and the like. Commercially available styrenic block copolymers include KRATON (styrenic block copolymer; available from the Kraton Chemical Company, Houston, TX), SEPTON (styrenic block copolymer; available from Kuraray America, Inc., New York, NY), VECTOR (styrenic block copolymer; available from TSRC Dexco Chemical Company, Houston, TX) can be used. Other suitable commercially available elastomers include ESTANE (polyurethane; available from Lubrizol, Inc, Ohio), PEBAX (polyether block amide; available from Arkema Chemicals, Philadelphia, PA), and HYTREL (polyester; available from DuPont, Wilmington, DE).

Semi-crystalline, or metallocene polyolefins are widely used in disposable absorbent articles. It is known that their performance depends on amount of crystallinity. The crystallinity decreases with decreasing stereoregularity, and the material shows more elastic behavior. A number of methods are known for controlling crystallinity, such as by introducing stereo-irregularity or by introducing a co-monomer. Some homopolyolefins and random copolymers, as well as blends of such random copolymers, known by tradenames Vistamaxx™ available from ExxonMobil and VERSIFY™ from Dow Corning, are synthesized based on this principle, and tend to show elastic performance. The polyolefin elastomer materials useful herein for making the elastically extensible material include, but are not limited to, any polymers or copolymers of polyolefins such as polyethylene and polypropylene. Suitable examples of elastomeric polypropylenes include an elastic random poly(propylene/olefin) copolymer, an isotactic polypropylene containing stereo-irregularity, an isotactic/atactic polypropylene block copolymer, an isotactic polypropylene/random poly(propylene/olefin) copolymer block copolymer, a stereoblock elastomeric polypropylene, a syndiotactic polypropylene block poly(ethylene-co-propylene) block syndiotactic polypropylene triblock copolymer, an isotactic polypropylene block regioirregular polypropylene block isotactic polypropylene triblock copolymer, a polyethylene random (ethylene/olefin) copolymer block copolymer, a reactor blend polypropylene, a very low density polypropylene (or, equivalently, ultra low density polypropylene), a metallocene polypropylene, and blends or combinations thereof. Suitable polypropylene polymers including crystalline isotactic blocks and amorphous atactic blocks are described, for example, in U.S. Pat. Nos. 6,559,262, 6,518,378, and 6,169,151. Suitable isotactic polypropylene with stereo-irregularity along the polymer chain are described in U.S. Pat. No. 6,555,643 and EP 1256594A1. Suitable examples include elastomeric random copolymers including propylene with a low level comonomer (e.g., ethylene or a higher alpha-olefin) incorporated into the backbone. Elastic polyethylene can be made similar to elastic polypropylene example, and can be used to make elastic laminate of the present invention.

In some configurations, two or more elastomers can be blended to achieve the desired elastic performance. For example, Styrenic block copolymer can be blended with polyolefin based elastomers, or polypropylene based elastomer can be blended with other polyolefin based elastomers.

The elastically extensible material may comprise modifying resins. Such modifying resins useful herein include, but are not limited to, unhydrogenated C5 hydrocarbon resins or C9 hydrocarbon resins, partially and fully hydrogenated C5 hydrocarbon resins or C9 hydrocarbon resins; cycloaliphatic resins; terpene resins; natural and modified rosins and rosin derivatives; coumarone indenes; polycyclopentadiene and oligomers thereof; polymethyistyrene or oligomers thereof; phenolic resins; indene polymers, oligomers and copolymers; acrylate and methacrylate oligomers, polymers, or copolymers; derivatives thereof; and combinations thereof. Modifying resins may also include alicyclic terpenes, hydrocarbon resins, cycloaliphatic resins, poly-beta-pinene, terpene phenolic resins, and combinations thereof. Useful C5 hydrocarbon resins and C9 hydrocarbon resins are disclosed in U.S. Pat. No. 6,310,154.

The elastically extensible material may comprise a variety of additives. Suitable additives including, but not limited to, stabilizers, and antioxidants, may be employed to prevent thermal, oxidative, and bio-chemical degradation of the elastically extensible material. Additives may account for about 0.01% to about 60% of the total weight of the elastically extensible material. In other suitable configurations, the elastically extensible material comprises from about 0.01% to about 10%, by weight, of additives.

The elastically extensible material may comprise various stabilizers and antioxidants that are well known in the art and include high molecular weight hindered phenols (i.e., phenolic compounds with sterically bulky radicals in proximity to the hydroxyl group), multifunctional phenols (i.e., phenolic compounds with sulfur and phosphorous containing groups), phosphates such as tris-(p-nonylphenyl)-phosphite, hindered amines, and combinations thereof. Proprietary commercial stabilizers and/or antioxidants are available under a number of trade names including a variety of Wingstay®, Tinuvin® and Irganox® products.

The elastically extensible material may comprise viscosity modifiers, processing aids, slip agents or anti-block agents. Processing aids include processing oils, which are well known in the art and include synthetic and natural oils, naphthenic oils, paraffinic oils, olefin oligomers and low molecular weight polymers, vegetable oils, animal oils, and derivatives of such including hydrogenated versions. Processing oils also may incorporate combinations of such oils. Mineral oil may be used as a processing oil. Viscosity modifiers are also well known in the art. For example, petroleum derived waxes can be used to reduce the viscosity of the slow recovery elastomer in thermal processing. Suitable waxes include low number-average molecular weight (e.g., 0.6-6.0 kilo Daltons) polyethylene; petroleum waxes such as paraffin wax and microcrystalline wax; atactic polypropylene; synthetic waxes made by polymerizing carbon monoxide and hydrogen such as Fischer-Tropsch wax; and polyolefin waxes.

A variety of fillers can also be used as additives to the elastic layer composition. Examples of suitable fillers include talc, calcium carbonate, carbon black, clay, and mica. The filler may be selected in combination with antioxidants to minimize impact on stability.

Elastic layer 38 may include at least one skin disposed on the elastically extensible material, the skin forming at least one of the first surface 38a or second surface 38b of elastic layer 38. Such skin is an extensible material and provides an outer surface to elastic layer 38 that has less tackiness than the underlying elastically extensible material. In some configurations, the skin layer may be made of plastically extensible material. In some configurations, the skin may qualify as an elastically extensible material, but will be less elastic than the underlying elastically extensible material. Accordingly, when compared to the elastically extensible material, the skin will have less recovery from the same amount of extension. Or in other words, when compared to the elastically extensible material, the skin will have a higher percent set from the same percent strain. The skin may aid in elastic layer 38 processability and is between about 1 μm and about 10 μm, or between about 3 μm and about 7 μm, or in some configurations, is about 5 μm, in thickness. In some configurations, the skin that overlays the elastically extensible material in elastic layer 38 is a polyolefin. Non-limiting examples of useful skin materials include metallocene polyethylene, low density polyethylene, high density polyethylene, linear low density polyethylene, very low density polyethylene, a polypropylene homopolymer, a plastic random poly(propylene/olefin) copolymer, syndiotactic polypropylene, metallocene polypropylene, polybutene, an impact copolymer, a polyolefin wax, polystyrene, filler particles such as calcium carbonate, and combinations thereof.

The elastic layer may be formed by any suitable method in the art, for example, by extruding molten thermoplastic and/or elastomeric polymers through a slit die and subsequently cooling the extruded sheet. Other non-limiting examples for making film forms include casting, blowing, solution casting, calendering, and formation from aqueous or, non-aqueous cast dispersions. One suitable method for obtaining the elastomer composition in the film form is by allowing polyolefin elastomers or other materials obtained in pellet form to be mixed and extruded by a high torque co-rotating twin-screw extruder, namely extrusion blending.

Particularly useful elastic layers and/or backsheet materials can include Elastipro 4008, Elastipro 4407, and DH215 elastic films commercially available from Berry Global (Mason, OH); CEX-826 elastic film commercially available from Fitesa (Terre Haute, IN); and MD5 elastic film available from Exen SA (Mendrisio, Switzerland). These exemplary elastic films include a single layer of elastically extensible material with a skin disposed on both surfaces of the material. Referring to FIG. 6A, such exemplary elastic films would have a skin providing first surface 38a and a second skin providing second surface 38b. However, other elastic films applicable to the backsheet only need to have a skin that provides first surface 38a or second surface 38b.

The constricting layer may have a thickness, prior to activation, of about 1 μm to about 30 μm, or from about 5 μm to about 20 μm, or from about 7 μm to about 10 μm. The constricting layer may have a basis weight of from about 1 gsm to about 20 gsm, or from about 7 gsm to about 10 gsm. In some configurations, the constricting layer may comprise polyolefin. Non-limiting examples of useful constricting layer materials include metallocene polyethylene, low density polyethylene, high density polyethylene, linear low density polyethylene, very low density polyethylene, a polypropylene homopolymer, a plastic random poly(propylene/olefin) copolymer, syndiotactic polypropylene, metallocene polypropylene, polybutene, an impact copolymer, a polyolefin wax, polystyrene, filler particles, and combinations thereof.

Particularly suitable backsheets may have certain physical properties such that the backsheet can stretch during activation, allowing the absorbent core structure to be fractured into discrete pieces, and can bring the pieces of absorbent material together to create a Relaxed Gap of about 280 microns or less. In some configurations, it may be further desirable that the backsheet have a certain force threshold that is greater than the peel force to remove the article from the undergarment in order reduce the risk of tearing during removal.

In some configurations, the backsheet may have an MD % Set at 100% Elongation of from about 1% to about 35%, as measured by the Hysteresis Method, or from about 5% to about 32%, or from about 10% to about 30%, or from about 12% to about 18%, specifically reciting all values within these ranges and any ranges created thereby. Without being limited by theory, it is believed that the relatively high recovery of such a backsheet will create a Relaxed Gap between the pieces of absorbent material of around 280 microns or less which can enable fluid communication between the pieces of absorbent material and improve the fluid handling properties. It is believed that a backsheet having an MD % Set at 100% Elongation of greater than 35% may not be able to bring the pieces of absorbent material into close enough proximity to neighboring pieces to enable efficient fluid communication and desired fluid handling properties.

In some configurations, the backsheet can have a MD % Set at 50% extension of less than 14% and/or an OD % Set at 50% extension of less than 16%, as measured according to the Hysteresis Method.

In configurations where the backsheet is a laminate comprising an elastic layer attached to a constricting layer, the elastic layer may comprise a film having an MD % Set at 100% Elongation of about 35% or less, as measured by the Hysteresis Method, or from about 1% to about 35%, or from about 3% to about 15%, or from about 5% to about 10%. In configurations where the backsheet is a laminate comprising an elastic layer attached to a constricting layer, the constricting layer may comprise a film having an MD % Set at 100% Elongation that is greater than the MD % Set at 100% Elongation of the elastic layer. In such a configuration, the constricting layer may comprise a film having a Backsheet Film MD Yield Force that is greater than about 5 N.

In some configurations, the backsheet may have a First Cycle MD Force at 100% extension of greater than 2.5 N, or greater than 5 N, as measured according to the Hysteresis Method. In some configurations, the backsheet may have a First Cycle MD Force at 100% extension of from about 2.5 N to about 100 N, as measured according to the Hysteresis Method. In some configurations, the backsheet may have a First Cycle MD Force at 50% extension of greater than 2 N, or greater than 5 N, as measured according to the Hysteresis Method. In some configurations, the backsheet may have a First Cycle MD Force at 50% extension of from about 2 N to about 100 N, as measured according to the Hysteresis Method. In some configurations, the backsheet may have a First Cycle MD Force at 20% extension of greater than 1 N, or greater than 6 N, as measured according to the Hysteresis Method. In some configurations, the backsheet may have a First Cycle MD Force at 20% extension of from about 1 N to about 100 N, as measured according to the Hysteresis Method.

In some configurations, the backsheet can have a Backsheet Film MD Yield Force of greater than about 1 N, or greater than about 2 N, or greater than about 5 N, or greater than about 6 N, as measured according to the Hysteresis Method.

The backsheet 30 may be positioned beneath or subjacent an outward-facing surface of the absorbent core structure 40 and may be joined thereto by any suitable attachment methods. For example, the backsheet 30 may be secured to the absorbent core structure 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In some configurations, it is contemplated that the absorbent structure core 40 is not joined directly to the backsheet 30.

The backsheet 30 may be impermeable or substantially impermeable by aqueous liquids (e.g., urine, menstrual fluid) and may be manufactured from a thin film, although other flexible liquid impermeable materials may also be used. As used herein, the term “flexible” refers to materials which are compliant and will readily conform to the general shape and contours of the human body. The backsheet 30 may prevent, or at least substantially inhibit, fluids absorbed and contained within the absorbent core structure 40 from escaping and reaching articles of the wearer's clothing which may contact the absorbent article 10, such as underpants and outer clothing. However, in some instances, the backsheet 30 may be made and/or adapted to permit vapor to escape from the absorbent core structure 40 (i.e., the backsheet is made to be breathable), while in other instances the backsheet 30 may be made so as not to permit vapors to escape (i.e., it is made to be non-breathable).

The backsheet 30 may have a basis weight of from about 14 gsm to about 80 gsm, or from about 20 gsm to about 60 gsm, or from about 22 gsm to about 45 gsm.

### Mechanical Processing

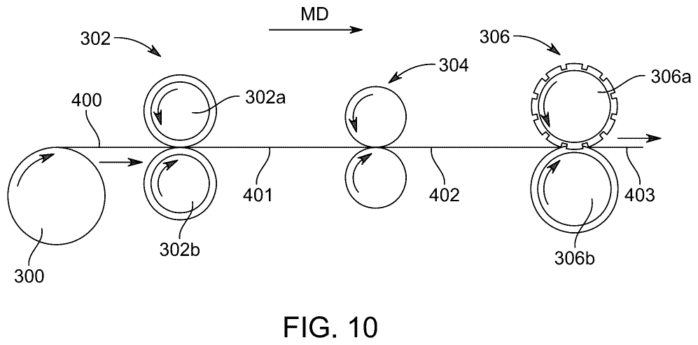

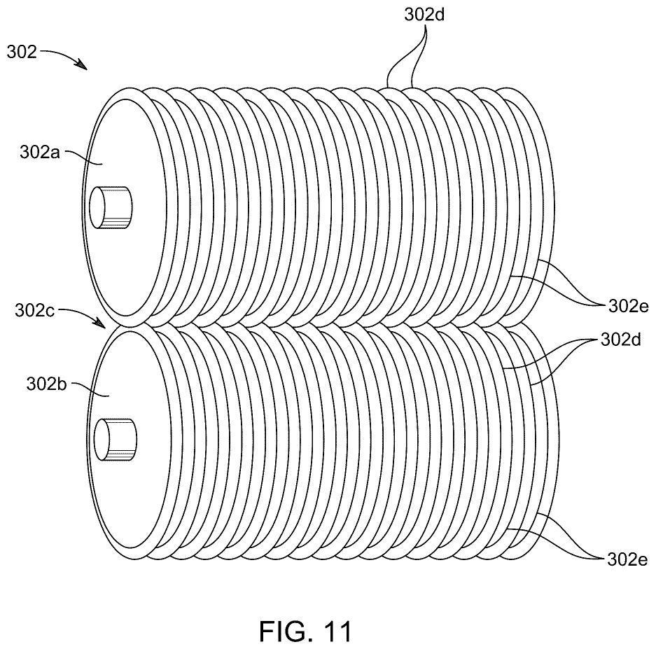

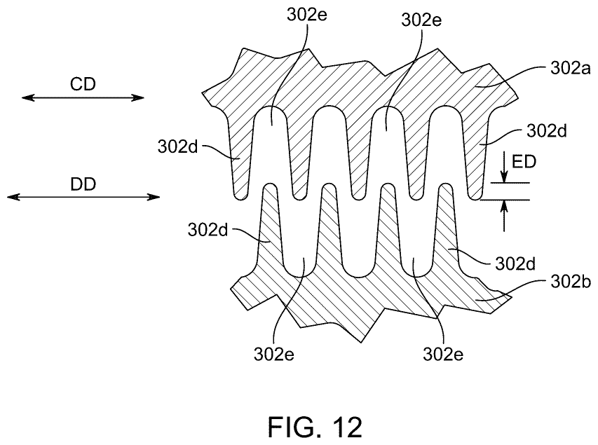

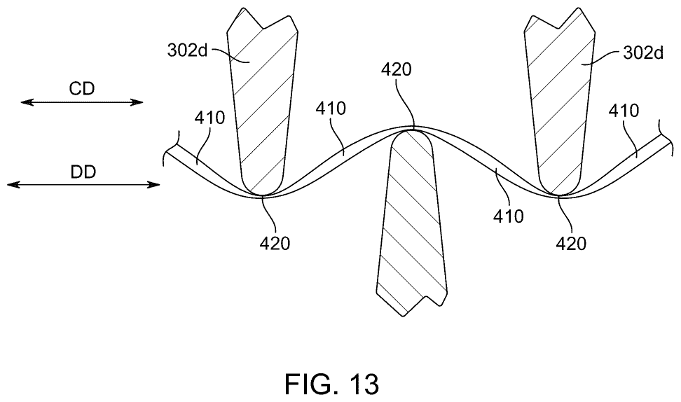

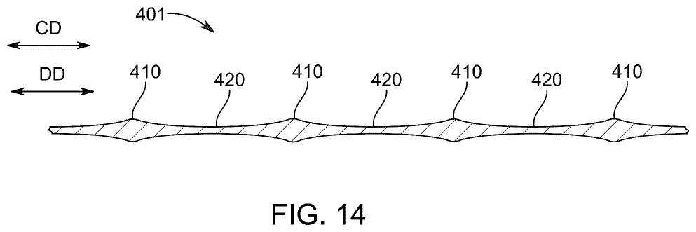

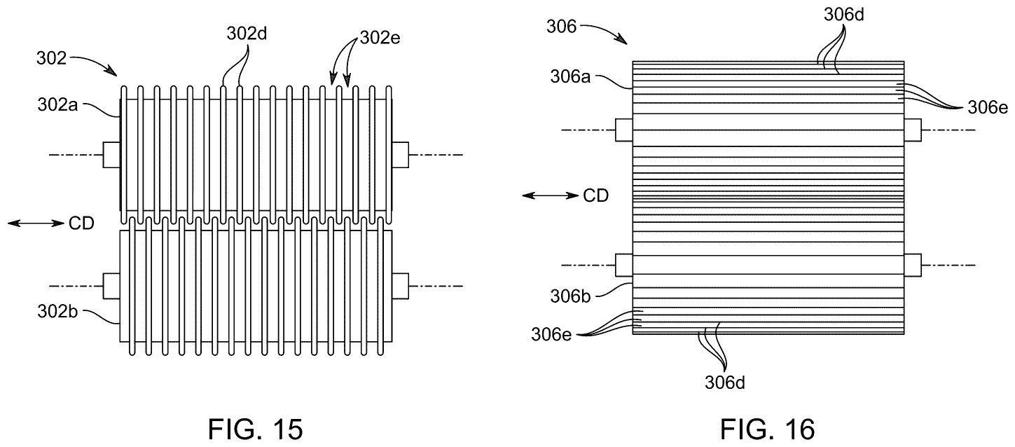

It has been learned that mechanically processing at least the absorbent core structure 40, or the absorbent core structure 40 together with one or both the topsheet 20 and backsheet 30, in the manner described herein, can provide a number of unexpected benefits. A process known as “incremental stretching” involves passing a web through a nip between a pair of rollers, the rollers having mating features that cause discrete, incremental sections of the web to be stretched across lines coincident with features of the rollers. Non-limiting examples of incremental stretching processes and equipment are disclosed in U.S. Pat. No. 6,383,431. It has been learned that this process may be applied to not only a single web layer, but a composite web of a plurality of layers including components of an absorbent core structure, to beneficial effect. Referring to FIGS. 10-15, for example, mating rollers 302a, 302b may be provided with respective circumferential ridges 302d separated by circumferential grooves 302e. The rollers may be configured such that the ridges of one engage with the grooves of the other to a desired engagement depth ED (FIG. 12). Upon passage of a layered composite web 400 being conveyed along a machine direction MD through a nip 302c between the engaged rollers, it will be stretched along a cross direction CD as it is forced to bend over the respective ridges 302d of each roller. If the engagement depth ED is suitably adjusted, one or more layer components of the composite web 400 can be caused to stretch beyond their yield points, to plastic deformation or even fracture, along machine direction lines, as the composite web 400 passes through the nip, resulting in a deformed composite web 401. (The roller configuration reflected in FIGS. 11-13 and 15 is sometimes known as a “ring rolling” configuration, or “ring rollers.”) Resulting less deformed, or undeformed, zones 410, and more-deformed zones 420 of this potential CD deformation or fracture in layer components of web 401 are schematically depicted in FIGS. 13 and 14. In configurations shown in FIGS. 12 and 13, the direction of stretch deformation DD is aligned or substantially parallel with the cross direction CD.

Referring to FIG. 16, in another configuration, deforming rollers 306a, 306b may be configured with mating/engaging ridges 306d and grooves 306e about their circumferential surfaces, that are parallel with their axes of rotation. In this configuration, the rollers resemble a pair of mating, elongate spur gears with their axes oriented in the cross direction CD. A web passing through the nip between these rollers will be stretched over the respective “gear teeth”, i.e., ridges 306d, substantially along a machine direction, rather than along a cross direction as described above and depicted in FIG. 11.

Referring now to FIG. 10, it has been learned that a composite web 400 including an absorbent core structure may be sequentially passed through the nips between two successive pairs of deforming rollers 302 and 306, causing the web to be incrementally stretched and/or fractured as described above, along two differing directions (in this particular non-limiting example, the machine direction and the cross direction). It has been learned that, following such deformation, compared with a similar structure that has not been so deformed, the absorbent core structure (1) is dramatically faster at acquiring and distributing fluid therealong and therethrough on the first gush; (2) exhibits comparatively greater absorption capacity per unit weight of absorbent material included, over the same time period; and (3) is dramatically more flexible and pliable, making it more capable of bending around and conforming to a non-ruled surface (i.e., body contours) and shifting with wearer body movement (i.e., moving and shifting along with underwear fabric, with wearer body movement). Without intending to be bound by theory, it is believed that an absorbent article having one or more layer components so processed will be dramatically more comfortable to a wearer/user of the product, and more effective at intercepting discharges of body fluids, as a result of greater conformability to body features and more rapid acceptance, distribution and absorption of fluid within its structure.

In the deformation process, the overall x-y plane surface area of the absorbent core structure may be increased by about 3% to about 25%, or from about 3% to about 15%, or from about 3% to about 5%. At the same time, the increase fluid acquisition and distribution speed capability and absorption capacity imparted to the article may enable the manufacturer to reduce or forego inclusion of particular material quantities and/or material layers (such as, for example, a separate acquisition/distribution layer), thereby enabling material cost savings as well as enable the manufacturer to offer a thinner, more comfortable and more discrete absorbent article (e.g., feminine hygiene pad) that performs at parity with thicker competing/comparable products, while being thinner, more discrete and more comfortable for the user. Because the pad is stretched and permanently deformed in the longitudinal and lateral directions (along x-y planar directions), the manufacturer may be able to reduce the overall x-y planar size of the absorbent structure and of the overall pad prior to deformation, and through the deformation process, cause the reduced-size pad structure to assume an expanded, final desired pad product size. The enhanced fluid acquisition speed and absorption capacity made possible via the deformation process makes more efficient use of the absorbent materials, and consequentially, a comparatively lesser quantity of absorbent material per pad is required to provide the desired fluid acquisition and absorption performance. Additionally, the deformation process described herein may eliminate the need to include apertures 43 (e.g., as shown in FIG. 2) through the absorbent structure, as described above, simplifying the manufacturing process.

It is believed that the enhanced liquid acquisition/distribution and absorption capabilities are attributable to the creation of added and/or larger internal voids (from fracturing of materials) within the absorbent core structure, along the lines of deformation. Further, when the composite web 400 passed between the deforming rollers includes not only an absorbent core structure but also one or both of a topsheet and backsheet components of an absorbent article, all of the layers may be deformed to varying extents depending upon their deformability or plastic extensibility, and all of the layers together as a composite can thereby be imparted with expanded size in the x-y directions, increased bidirectional extensibility, flexibility, and pliability.

This deformation along two directions is referred to herein as “bidirectional” deformation. Importantly, the bidirectionally-deformed composite web has enhanced capability to bend around and more closely conform to non-ruled, curving-contour surfaces such as the surfaces of human body features. The extent of this conformability is reflected in measurements that may be taken using the Conformability Force Measurement Method described below. In some configurations, the absorbent article described herein may exhibit a Conformability Force of from about 140 N/m to about 1500 N/m, or from about 150 N/m to about 1000 N/m, or from about 225 N/m to about 800 N/m. In contrast, current feminine hygiene pads exhibit a Conformability Force of greater than 1600 N/m, with some even greater than 5100 N/m. Without being limited by theory, it is believed that products that exhibit a Conformability Force of from about 140 N/m to about 1500 N/m are highly flexible and can move with the panty during wear, which can result in a more comfortable and/or closer fit to the body.

Referring to FIGS. 1, 5A-5C, 7, and 14, the materials of the respective topsheet 20 and backsheet 30 may be selected and/or manufactured and/or formulated for having properties, and the engaging features and engagement depths of the deforming rollers may be configured and adjusted such that, for example, the topsheet and/or backsheet only stretch elastically but not plastically, or alternatively plastically but not to fracture/failure/breakage, in deformed zones 420 in the areas corresponding to the ridges of the deforming rollers, as they pass through the respective nips between the deforming rollers. In combination, the material(s)/layer(s) of the absorbent core structure 40 may be selected and/or manufactured and/or formulated for having properties such that they will be stretched plastically or even to breakage/fracture, in an orderly manner and pattern in regions 420, reflecting the patterns of ridges and grooves on the deforming rollers. Preferably, the topsheet and backsheet are selected and/or manufactured and/or formulated such that they will be plastically (or permanently) stretched/deformed in zones 20s, 30s, substantially corresponding with lines of deformation 50, but not fractured in the deforming process, while the absorbent structure material 40 is stretched to fracture to create gaps 40s, also substantially corresponding with lines of deformation 50. In such a configuration, the combination of materials to form the absorbent article, as a composite, can be imparted with bidirectional extensibility and substantially enhanced pliability and body conformability.

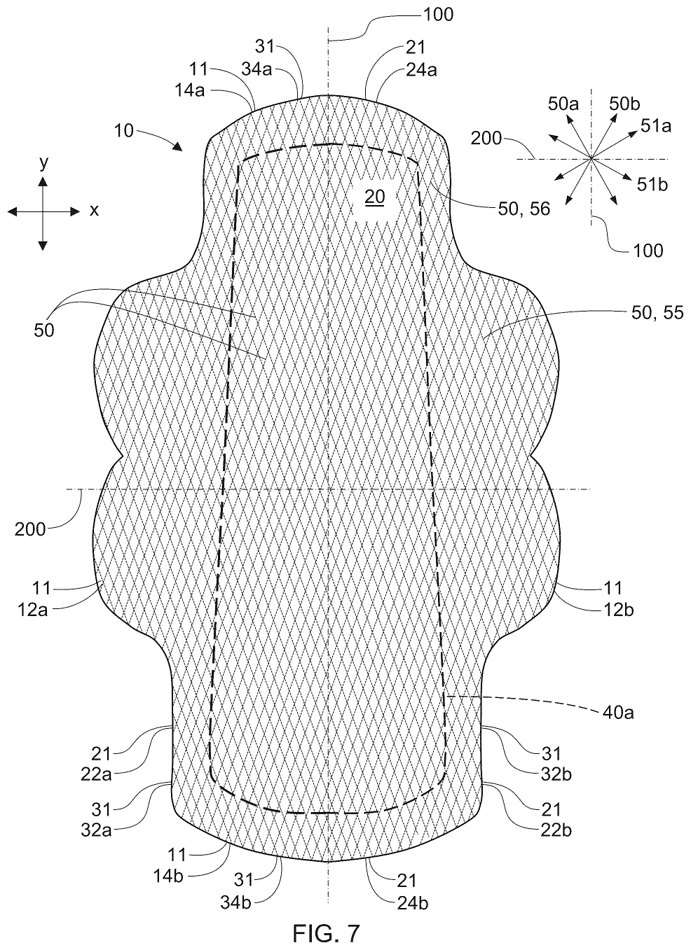

When the topsheet and backsheet are only plastically stretched to greater x-y dimensions but not fractured, and one or more of the absorbent core structure component(s)/layer(s) are stretched to fracture so as to form discrete fractured pieces 40p thereof, gaps 40s between fractured edges of the pieces substantially along lines of deformation 50 open up, thereby providing and opening fluid pathways through the absorbent structure, and providing added surface area of absorbent material, which fluid may contact so as to be absorbed more rapidly, as compared with an absorbent layer component that has not been so fractured. A non-limiting example of an absorbent article in the form of a feminine hygiene pad having undergone such bidirectional deformation along the CD and MD is schematically depicted in FIG. 1. The longitudinal/y and lateral/x directions of the pad correspond with the CD and MD directions of deformation, along lines of deformation 50, and are arranged at or approximately at 90 degrees from each other. Another non-limiting example of an absorbent article in the form of a feminine hygiene pad having undergone such bidirectional deformation, along angles oblique to the CD and MD, is schematically depicted in FIG. 7.

Referring to FIGS. 1 and 7, the absorbent article 10 comprises sides 11, for example a first side, a second side, a third side, and a fourth side. Absorbent article 10 may comprise a first longitudinal side 12a that extends in a direction substantially parallel to the longitudinal axis 100 and a second longitudinal side 12b opposite the first longitudinal side 12a. Absorbent article 10 may also comprise a first lateral side 14a that extends in a direction substantially parallel to the lateral axis 200 and a second lateral side 14b opposite the first lateral side 14a. As shown in FIG. 7, in some configurations, the absorbent article may comprise a first plurality of lines of deformation 55 extending in a first direction substantially perpendicular to a first stretch direction 51a and a second plurality of lines of deformation 56 extending in a second direction substantially perpendicular to a second stretch direction 51b. In some configurations, at least a portion of the first plurality of lines of deformation 55 extend from a first side of the absorbent article 10 to a second side of the absorbent article 10. In some configurations, a portion of the first plurality of lines of deformation 55 may extend from the first longitudinal side 12a to at least one of the second lateral side 14b and the second longitudinal side 12b. In some configurations, a portion of the second plurality of lines of deformation 56 may extend from the first longitudinal side 12a to at least one of the first lateral side 14a and the second longitudinal side 12b.

Referring to FIGS. 1 and 7, the topsheet 20 and the backsheet 30 may comprise sides 21, 31, respectively. For example, topsheet 20 may comprise a first side, a second side, a third side, and a fourth side, and the backsheet 30 may comprise a first side, a second side, a third side, and a fourth side. The topsheet 20 may comprise a first longitudinal side 22a that extends in a direction substantially parallel to the longitudinal axis 100 and a second longitudinal side 22b opposite the first longitudinal side 22a. The topsheet 20 may also comprise a first lateral side 24a that extends in a direction substantially parallel to the lateral axis 200 and a second lateral side 24b opposite the first lateral side 24a. The backsheet 30 may comprise a first longitudinal side 32a that extends in a direction substantially parallel to the longitudinal axis 100 and a second longitudinal side 32b opposite the first longitudinal side 32a. The backsheet 30 may also comprise a first lateral side 34a that extends in a direction substantially parallel to the lateral axis 200 and a second lateral side 34b opposite the first lateral side 34a. In some configurations, the topsheet 20 and the backsheet 30 may each comprise plastically stretched zones 20s, 30s disposed substantially along the first plurality of lines of deformation 55 and the second plurality of lines of deformation 56. In some configurations, the topsheet 20 may comprise a plurality of plastically stretched zones 20s that extend continuously across the entire topsheet 20. In some configurations, a portion of the plastically stretched zones 20s of topsheet 20 may extend continuously from a first side of the topsheet 20 to a second side of the topsheet 20. For example, a portion of the plastically stretched zones 20s of topsheet 20 may extend continuously from the first longitudinal side 22a to the second longitudinal side 22b. In some configurations, a portion of the plastically stretched zones 20s of topsheet 20 may extend continuously from the first lateral side 24a to the second lateral side 24b. In some configurations, the backsheet 30 may comprise a plurality of plastically stretched zones 30s that extend continuously across the entire backsheet 30. In some configurations, a portion of the plastically stretched zones 30s of backsheet 30 may extend continuously from a first side of the backsheet 30 to a second side of the backsheet 30. For example, a portion of the plastically stretched zones 30s of backsheet 30 may extend continuously from the first longitudinal side 32a to the second longitudinal side 32b. In some configurations, a portion of the plastically stretched zones 30s of backsheet 30 may extend continuously from the first lateral side 34a to the second lateral side 34b.

For feminine hygiene pads having an open-celled absorbent foam layer (e.g., a HIPE foam layer as described above) forming part or substantially all of the absorbent core structure 40, in order to provide the desired overall pad flexibility and effective fluid passageways, it may be desired that the deforming rollers be configured and dimensioned to impart plastic deformation to the topsheet and backsheet and fracture of the absorbent foam layer such that pieces 40p have an average x-y planar size, across the entirety or substantially the entirety of the absorbent structure, no greater than about 15 mm, or no greater than about 10 mm, or no greater than about 7 mm, or no greater than about 5 mm. (For purposes herein, the “x-y planar size” of a piece 40p is its greatest x-y planar dimension.) In some configurations, the average x-y planar size of the foam pieces 40p may be from about 1.5 mm to about 15 mm, or from about 2 mm to about 5 mm, specifically reciting all values within these ranges and any ranges created thereby. In some configuration, the foam pieces 40p may have an x-y planar shape of a diamond.

After deformation, the backsheet as described herein, to which the pieces 40p are attached, can help to carry the pieces 40p closer together such that the gaps 40s formed between neighboring pieces have an x-y planar Relaxed Gap size of about 280 microns or less, or from about 10 microns to about 280 microns, or from about 50 microns to about 200 microns, or from about 75 microns to about 150 microns, or any sub-range therewithin. (For purposes herein, the “x-y planar gap size” of a gap 40s is the x-y planar dimension of the space between adjacent pieces, measured along a direction perpendicular to the corresponding line of deformation 50.) Foam pieces 40p may be separated from neighboring pieces by a Stretched Gap size of from about 500 microns to about 3 mm, or from about 800 microns to about 1 mm. Relaxed Gap can be measured according to the SEM Imaging Method and Stretched Gap can be measured according to the Stretched Gap Measurement Method.

Without being limited by theory, it is believed that a backsheet having the properties described herein can bring the discrete pieces of absorbent material close together after deformation, and form the gap size 40s described above. As a result of the relatively small gap size, the impression of a more continuous product that appears more protective and/or of higher quality can be created. In addition, fluid rewet values may be improved because less free fluid will be present in the channels between the pieces of absorbent material and/or on the top surface of the absorbent article (providing a dry feeling to the consumer).

FIGS. 5A-5C schematically depict non-limiting examples of potential effects resulting from incremental stretching of a composite web including topsheet material, backsheet material and absorbent structure material, along the lateral direction of the pad depicted in FIG. 1. Topsheet 20 may be imparted with plastically stretched zones 20s, and backsheet 30 may be imparted with plastically stretched zones 30s, in relatively orderly configurations along lines of deformation 50 (FIG. 1), along which plastic deformation of the topsheet material and/or backsheet material has occurred at the regions corresponding to the roller teeth or ridges in the nip between the deforming rollers. Topsheet 20 and backsheet 30 may comprise zones of less or substantially no deformation 20u, 30u, respectively, disposed intermediate the plastically stretched zones. Absorbent core structure 40 may be imparted with relatively orderly lines of plastic strain deformation or, preferably, fracture, to form gaps 40s. In some configurations, an absorbent core structure 40 may include additional acquisition and/or distribution layers imparted with relatively orderly lines of strain deformation or even fracture to form strained regions or even gaps. In some configurations, additional layers may include nonwoven web materials, in which portions of an absorbent foam precursor have been integrated into the fibrous matrices thereof and subsequently cured or polymerized into foam structure, as suggested in, e.g., US2017/0119587; US2017/0119596; US2017/0119597; US2017/0119588; US2017/0119593; US2017/0119594; US2017/0119595; and US2017/0199598.

The size of the gaps 40s between foam pieces 40p in an article in which an absorbent foam as described herein forms or is a component of absorbent structure 40 may be adjusted via configuration of the deforming rollers. One aspect of such configuration that is particularly effective is the engagement depth ED of the respective, cooperating/meshing deforming ridges 302d and grooves 302e (see FIG. 12). Greater engagement depth ED will cause greater deformation of the topsheet and backsheet and thereby effect relatively larger gaps 40s, while lesser engagement depth ED will cause lesser deformation of the topsheet and backsheet and thereby effect relatively smaller gaps 40s. Deforming roller engagement depth should be limited, however, so that topsheet 20 and backsheet 30 are not stretched to failure along lines of deformation 50, and/or so that potentially negative consumer perceptions of quality of the article (e.g., that the article is too flimsy or insubstantial, or damaged) do not arise. The size of the gaps 40s between foam pieces 40p may also be adjusted by selecting a backsheet having a percent set value as described herein.

Referring now to FIG. 7, in some configurations, the absorbent article may be imparted with lines of deformation 50 that are oblique to the lateral axis 200 and longitudinal axis 100. To effect this result, in some examples, the combination web or an assembled article may be passed between machine-direction ring rollers 302a, 302b, like those shown in FIGS. 11-13 and 15, by being fed therethrough in two successive steps, along two differing, oblique directional orientations relative the machine direction. In other examples (not illustrated), the combination web or assembled article progressing along a machine direction may be successively, intermittently immobilized and compressed or stamped along a z-direction, between pairs of flat deforming plates, which bear ridges and grooves suitably configured to incrementally stretch the web or article along oblique directions. However, for purposes of incrementally stretching a combination web along two differing directions oblique to a machine direction of continuous manufacturing, such methods may be found to be cumbersome and inefficient, as compared with using helical deforming rollers as described herein.

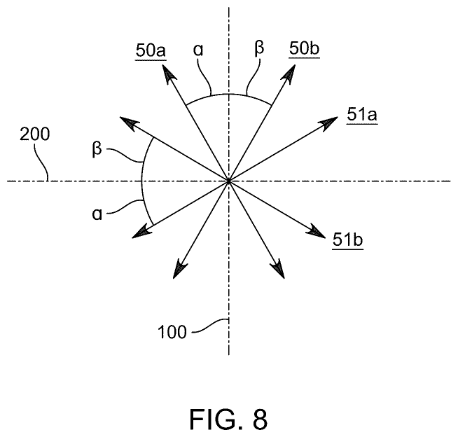

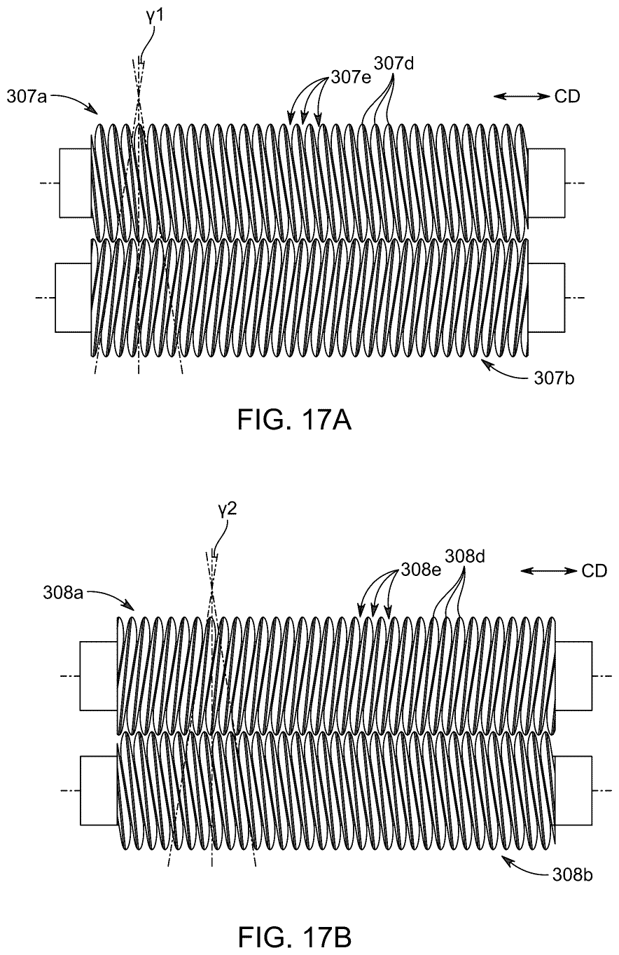

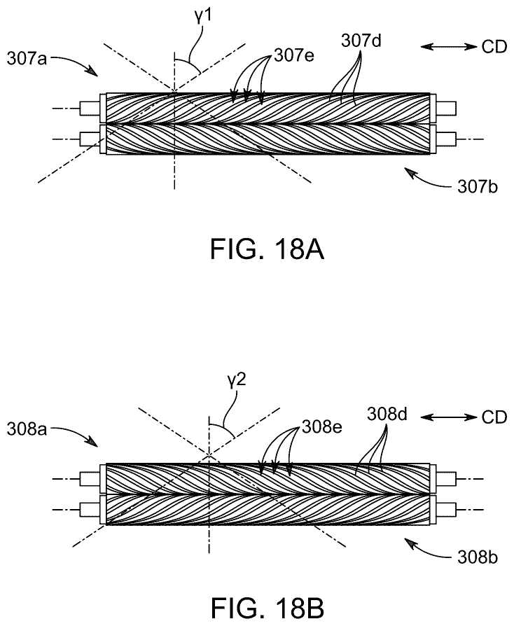

Alternatively, the deforming rollers used may be imparted with configurations resembling those of a mating/meshing pair of helical gears, spiral gears or worm gears (collectively herein, deforming rollers having a “helical” configuration of alternating grooves and ridges), as suggested in FIGS. 17A, 17B, 18A and 18B. As reflected in FIGS. 17A, 17B, 18A and 18B (and also referring to FIGS. 7 and 8), a first pair of such helical deforming rollers 307a, 307b may be configured to mesh and have helix angles γ1 selected, upon passage of an article (or precursor combination web) through a nip therebetween, to impart the article 10 with lines of deformation 50 substantially parallel with one of line of deformation directions 50a, 50b, by incrementally straining the article along one of strain directions 51a, 51b. Following passage through the nip between rollers 307a, 307b, the article (or precursor combination web) may be passed through a second nip between a second pair of deforming rollers 308a, 308b (FIGS. 17B, 18B), which also may be configured to be helical and be adapted to mesh and be configured with helix angles γ2 selected, upon passage of the article (or precursor combination web) through the nip therebetween, to impart the article 10 with lines of deformation 50 substantially parallel with the other of line of deformation directions 50a, 50b, by incrementally straining the article along the other of strain directions 51a, 51b.

With such a configuration, the resulting deformation of the web passing through a nip therebetween will occur along lines of deformation 50 that are oblique relative the MD and CD on the web, with respect to the machine and cross directions, along an angle of deformation imparted as a result of the helix angles of the helical ridges 307d, 308d and meshing helical grooves 307e, 308e along the outer circumferences or outer radial edges of the rollers. Two successive pairs of such helical deforming rollers having differing or oppositely-oriented helix angles may be arranged along a processing line to successively strain and deform a composite web along two differing directions and thus impart bidirectional deformation to the article or a precursor web thereof, for example, as suggested in FIG. 7. It may be preferred that the helix angles of the successive helical deforming roller pairs be selected such that the angles α and β formed at the intersection of resulting lines of deformation 50 with the longitudinal axis of the article, be substantially equal, for purposes of imparting stretch/elongation/conformability characteristics to the article, and appearance, that is substantially symmetrical about and/or aligned with the longitudinal axis.

Referring back to FIGS. 7 and 8, helical deforming rollers may be configured to impart bidirectional strain and deformation along strain directions 51a, 51b that are oblique with respect to the longitudinal/x and lateral/y directions of an absorbent article 10 such as a feminine hygiene pad. For a feminine hygiene pad, it has been learned that a bidirectionally deformed pad with lines of deformation 50 parallel to line of deformation directions 50a, 50b, which are substantially perpendicular to strain directions 51a, 51b, which strain directions are oriented at angles α and β with respect to the lateral direction, respectively, more readily shifts and accommodates a wearer's body (e.g., walking) movements well, when adhered to the inside of the wearer's underwear in the crotch region thereof, when angles α and β are each about 5 degrees to about 85 degrees, or about 15 degrees to about 70 degrees, or about 30 degrees to about 60 degrees. Correspondingly, it is preferred that line of deformation directions 50a, 50b each be about 5 degrees to about 85 degrees, or about 15 degrees to about 70 degrees, or about 30 degrees to about 60 degrees, with respect to the longitudinal axis of the article. All subranges within these ranges are contemplated herein.

Adhered to the inside surface of the wearer's underwear, the pad may be better able to shift and move along with the fabric of the underwear as the wearer moves about. This results in a dramatic improvement to wearer comfort.

It has also been learned that such strain directions, being oblique with respect to the machine direction of movement of the web through the nip, are less likely to be destructive of a nonwoven web material, than a strain along a direction that is parallel or perpendicular to the machine direction (as would be imparted by rollers such as those shown in FIGS. 11-16). This is believed to be a previously unrecognized synergistic benefit, resulting from the typical machine direction bias of fibers in nonwoven web materials, particularly spunbond nonwoven web materials. Spunbond nonwoven web materials typically have a machine direction bias as a result of the manner in which they are manufactured, i.e., by deposition of spun fibers upon a forming belt moving along a machine direction. (Herein, “machine direction bias,” with respect to the fibers forming a nonwoven web, means that a majority of the fibers, as situated in the web and unstretched, have lengths with machine direction vector components that are greater than their cross direction vector components.) As a result of the manner in which articles 10 and their component web materials are typically manufactured, the nonwoven web components typically have a machine direction bias that coincides with the longitudinal (y) direction of the article. When the deformation directions 51a, 51b are oblique with respect to the machine/longitudinal directions as suggested in FIGS. 7 and 8, fibers are less likely to be pulled directly along their lengths in the machine direction or separated in the cross direction, and as a result, are less likely to be stretched along their lengths beyond their limits and break, and less likely to be separated along a cross direction, which can result in unwanted change or damage to the structural integrity and quality of the web material.

It is contemplated that a pair of deforming rollers may be configured to impart bidirectional deformation to a composite web in a single pass through the nip, i.e., wherein features of the rollers are configured to cause incremental stretching of the composite web simultaneously in two differing directions, including directions parallel and orthogonal to the machine direction, or oblique to the machine direction, as described above. It may be preferred, however, to impart stretching along two differing directions, successively, via two successive pairs of deforming rollers. This may enable better control over the stretching process along each direction, and reduce chances of undesired lines or paths of fracture in the absorbent structure, or damage to components of the web.

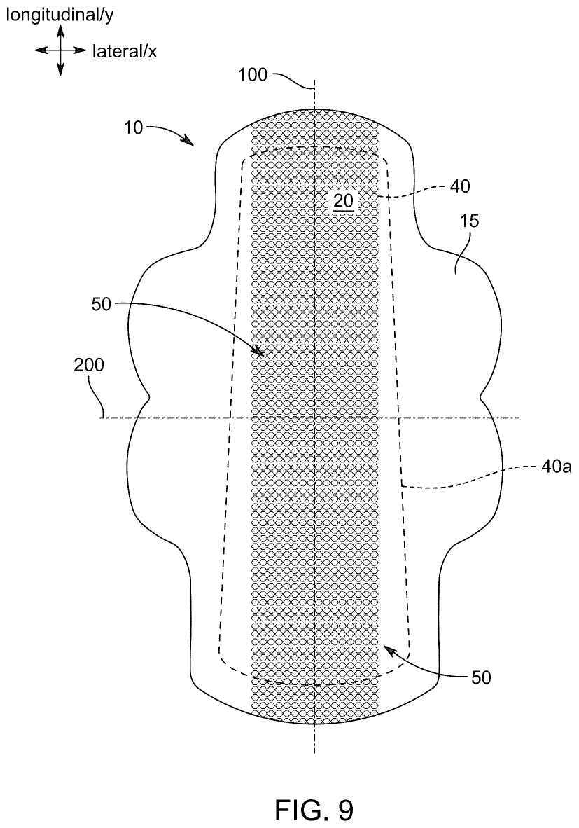

It is also contemplated that one or more pairs of deforming rollers may be configured to deform only discrete regions or zones of a composite web, while leaving adjacent regions or zones undeformed. By way of non-limiting example depicted in FIG. 9, an absorbent article 10 in the form of a feminine hygiene pad may be bidirectionally deformed only in a defined zone or region (in the non-limiting example shown in FIG. 9, the center region shown with oblique lines of deformation 50), while the remaining areas are left undeformed. The bidirectional deformation may be imparted along directions 50a, 50b and 51a, 51b angles α and β, as described above. In the examples shown in FIG. 9, a center region is deformed, and side regions including wings 15 are left undeformed. Regions of deformation and regions to be left undeformed may be configured for various effects.

It may be preferred, however, to bidirectionally deform the entirety of the x-y area of the layered materials forming the article 10 (as suggested in FIGS. 1 and 7), with no breaks or discontinuities in the bidirectional deformation pattern like, for example, that shown in FIG. 9, wherein a portion or portions have been left undeformed. This is because a discontinuity or interruption in the bidirectional deformation pattern can result in discontinuity of the pliability and flexibility imparted by the deformations, compromising the ability of the absorbent article 10 to closely conform to a wearer's body features and shift with the fabric of the wearer's underwear and/or the wearer's body movements.

Referring again to FIGS. 1 and 5A-5C, as discussed above, the bidirectional deformation process may be configured to fracture material of the absorbent core structure 40 along lines of deformation 50 in an orderly manner, into a plurality of discrete pieces 40p. Where, for example, the material of absorbent core structure 40 includes a layer of material that is relatively inelastic or brittle in tension (such as, for example, absorbent foam of suitable composition as described herein), the process, suitably configured, will fracture the structure 40 in an orderly manner to create roughly evenly-sized discrete pieces 40p separated by gaps 40s, divided along the lines of deformation 50.

In order to prevent the discrete pieces 40p, following fracture, from becoming dislodged and dislocated within the structure (i.e., to hold them in place substantially in their relative pre-fracture positions), it may be desirable to include structure to hold them in place, within the enveloped space between the topsheet and the backsheet. For this purpose, deposits 45 of a suitable adhesive/glue material may be disposed in locations between one or both of the wearer-facing and outward-facing interfaces between the absorbent core structure 40 and the topsheet, and backsheet, respectively, or between the absorbent core structure 40 and other interlayered components such as, for example, distribution layer(s). Particularly for adhesive that may be disposed between the wearer-facing surface of the absorbent core structure 40 and the topsheet 20, the deposits 45 may be applied via controlled spraying in the manner discussed above, so as not to create an occlusive film of adhesive, but to bond the respective materials together at discrete locations corresponding to deposited glue droplets, while avoiding creation of a fluid barrier, and leaving the absorbent core structure 40 effectively unoccluded on its wearer-facing surface. In some configurations, however, it may be deemed suitable or even preferred that an application of adhesive disposed between the outward-facing surface of the absorbent core structure 40 and the backsheet be more extensive, continuous or even substantially film-like, since occlusion at or on the outward-facing surface of the absorbent structure (e.g., a layer of absorbent foam) may be of less concern; and such more extensive or continuous application may better serve to hold fractured pieces 40p of absorbent core material in place, following deformation as described herein. Thus, the adhesive deposits may be disposed at both the upper and lower surfaces of the absorbent core structure 40 or a layer component thereof to provide a more cohesive overall pad structure and minimize chances that pieces 40p can be dislodged and dislocated within the structure.

Preferably, the adhesive deposits are applied across a majority of the x-y plane surface area of one or both of the wearer-facing surface and the garment-facing surfaces of the absorbent core structure 40. To reduce chances of a stiffening effect resulting from application of the adhesive/glue between respective subjacent/superadjacent layers of the pad, it may be desired that the adhesive/glue material selected be of an elastic and/or resilient formulation. In some configurations, the adhesive may be a pressure sensitive adhesive with a relatively long open time. Suitable adhesives may have a relatively high elastic modulus (G′) to withstand the forces applied during mechanical processing as the materials are stretched. Adhesives utilizing a styrene/isoprene/styrene (SIS) building block may be preferred. One suitable example may be an adhesive of the designation H2031-C5X, a product of Bostik, a division or subsidiary of the Arkema Group, Columbes, France.

In some configurations, adhesive may be disposed between the wearer-facing surface of the absorbent core structure 40 and the topsheet as shown and described in FIGS. 5A-5C. In some configurations, adhesive may be disposed between the wearer-facing surface of the absorbent core structure 40 and the topsheet at a basis weight of from about 15 gsm to about 35 gsm, or from about 18 gsm to about 32 gsm, or from about 20 gsm to about 30 gsm, specifically reciting all values within these ranges and any ranges created thereby. In some configurations, adhesive may be disposed between the outward-facing surface of the absorbent core structure 40 and the backsheet at a basis weight of from about 15 gsm to about 35 gsm, or from about 18 gsm to about 32 gsm, or from about 20 gsm to about 30 gsm, specifically reciting all values within these ranges and any ranges created thereby. Consumer testing has surprisingly revealed that when the basis weight of the adhesive between the topsheet and absorbent core structure and/or between the backsheet and absorbent core structure is below 15 gsm, respectively, the pieces of foam can become dislodged and dislocated within the absorbent article during wear, creating a non-uniform distribution of absorbent material which can negatively impact comfort and/or fluid handling performance.

Because the elastic layer 38 includes both an elastically extensible material and at least one skin disposed on the elastically extensible material, and because these materials have different elasticity and recovery properties, the incremental stretching process will physically alter these materials differently. During activation, the skin and the elastically extensible material are similarly stretched (i.e., put under similar strain). However, after stretching, the skin and the elastically extensible material will retract and recover differently (i.e., have different set values). In comparison with the elastically extensible material, the skin is either less elastic or plastic and therefore will have less recovery after stretching, a.k.a., a higher set value. The skin is also much thinner than the elastically extensible material, so when the thicker elastically extensible material retracts and recovers after activation stretching, it will force the attached skin to retract with it. But because the skin cannot recover as much as the elastically extensible material, the skin buckles and wrinkles. Accordingly, the cross-sectional profile and the top view appearance of elastic layer 38 are modified after activation.

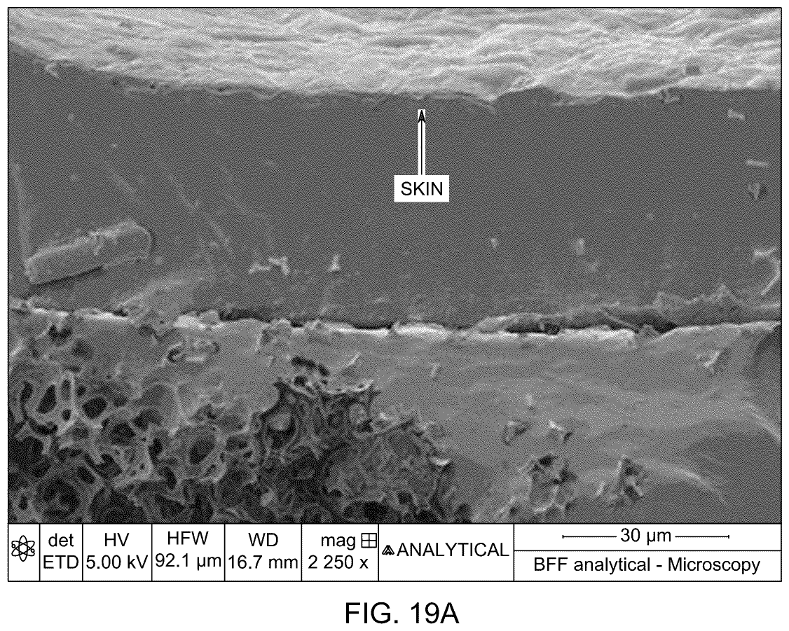

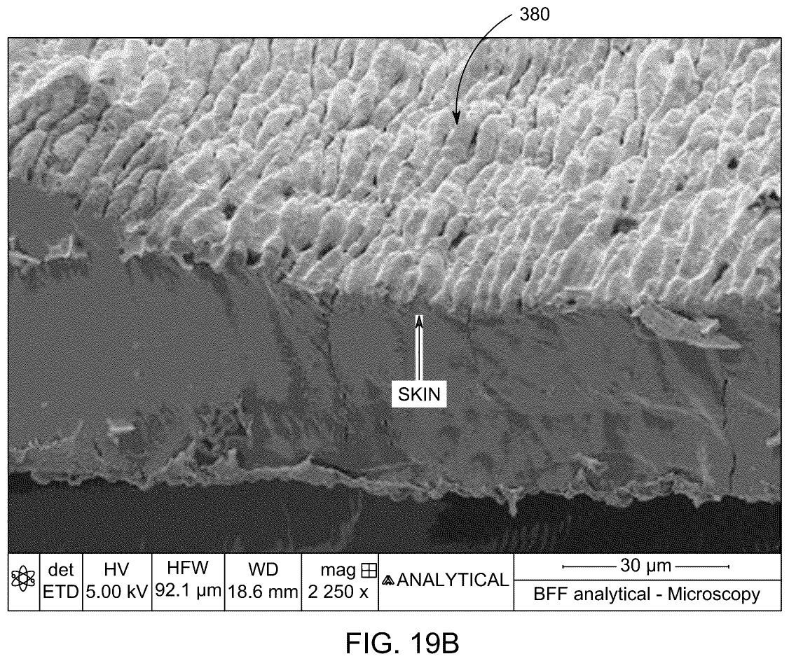

In some configurations, a portion of the backsheet located in the lines of deformation 50 described above may comprise a plurality of micro-wrinkles. In particular, it was found that after activation, the skin of the elastic layer located in the lines of deformation 50 include a plurality of micro-wrinkles having hills and furrows. FIGS. 19A and 19B are SEM photomicrographs taken at 2250× magnification showing a tilted cross-section view of a portion of an elastic layer after activation. It is noted that in FIGS. 19A and 19B, the absorbent core material underlying the elastic layer has been substantially removed for ease of viewing. These SEM photomicrographs, as well as the other SEM photomicrographs were taken with a scanning electron microscope (FEI Quanta 450). The skin is the thin strip of contrasting material at the top of the cross-section, with the thicker elastically extensible material below the skin. FIG. 19A shows that after activation, the skin of the elastic layer does not have micro-wrinkles in an area outside of the line of deformation. In contrast, FIG. 19B show that after activation, the skin of the elastic layer located within a line of deformation includes a plurality of micro-wrinkles 380. As visible on the top surface of the elastic layer shown in FIG. 19A, one or more random hills and/or furrows may be present within a particular length of cross-sectional view of an elastic layer that is outside of a line of deformation. These random hills and/or furrows are due to irregularities in the surface of the elastic layer and/or skin. Such random hills and/or furrows should not be confused with the hills and furrows of the micro-wrinkles that are intentionally formed through the mechanical activation process. As used herein, the term “micro-wrinkle” refers to a small fold, ridge or crease that has a height of about 10 microns or less. Without being limited by theory, it is believed that when the backsheet is a unitary structure, as shown in FIG. 6B, micro-wrinkles will be formed in the constricting layer in areas located in the lines of deformation.

Panty fastening adhesive applied to the garment-facing surface of the backsheet may flow over the hills and into the furrows of the micro-wrinkles in the lines of deformation. Accordingly, panty fastening adhesive may be disposed in the furrows of the micro-wrinkles in the lines of deformation and may allow for more contact surface area between the backsheet and the adhesive, leading to a stronger bond between the backsheet and the undergarment.

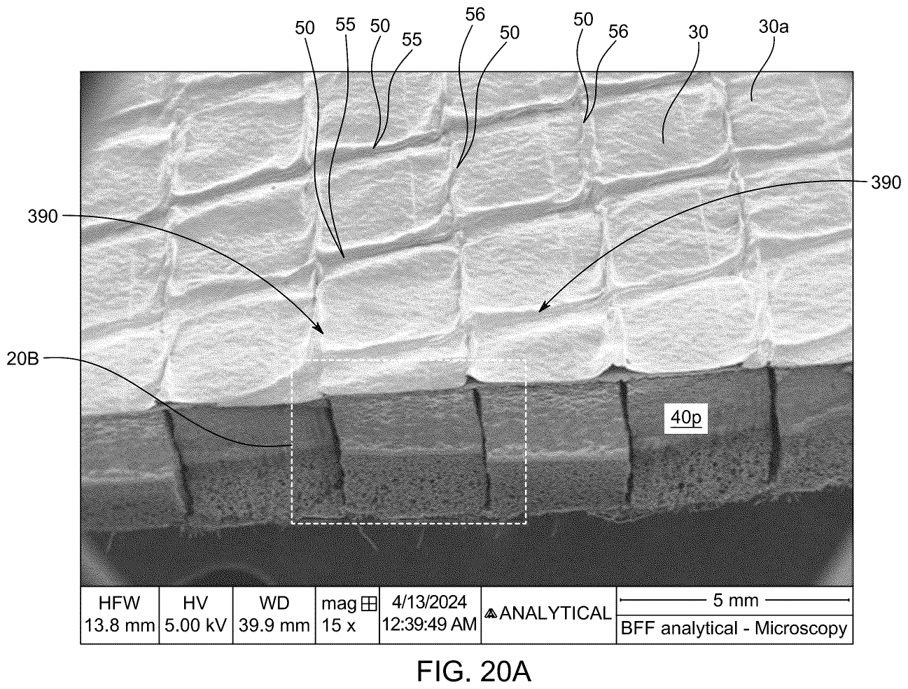

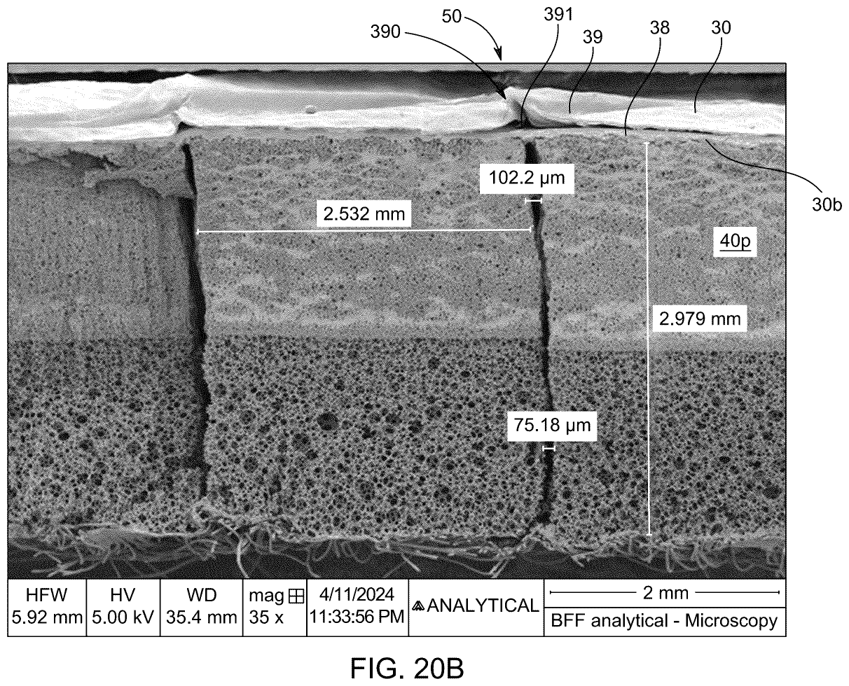

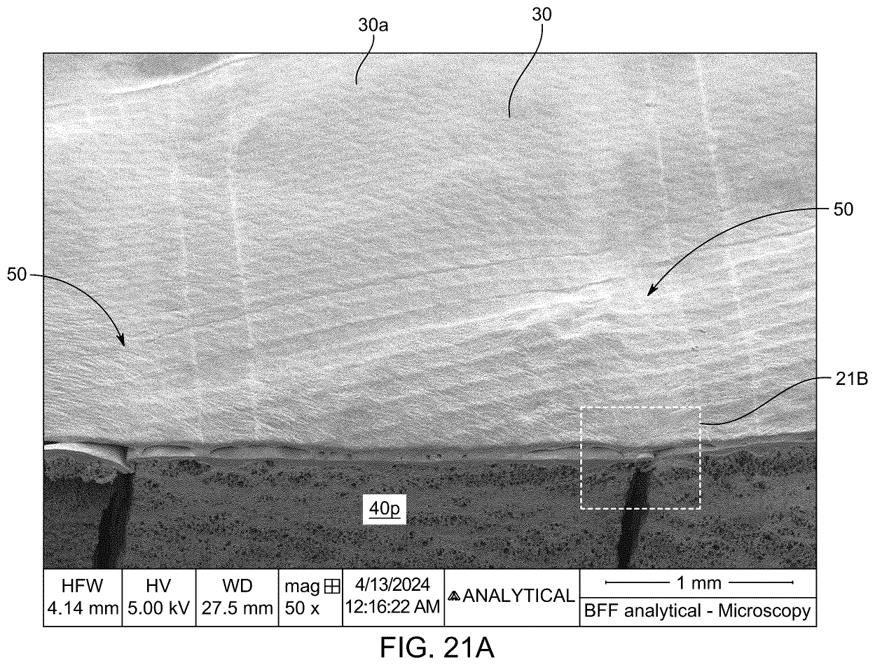

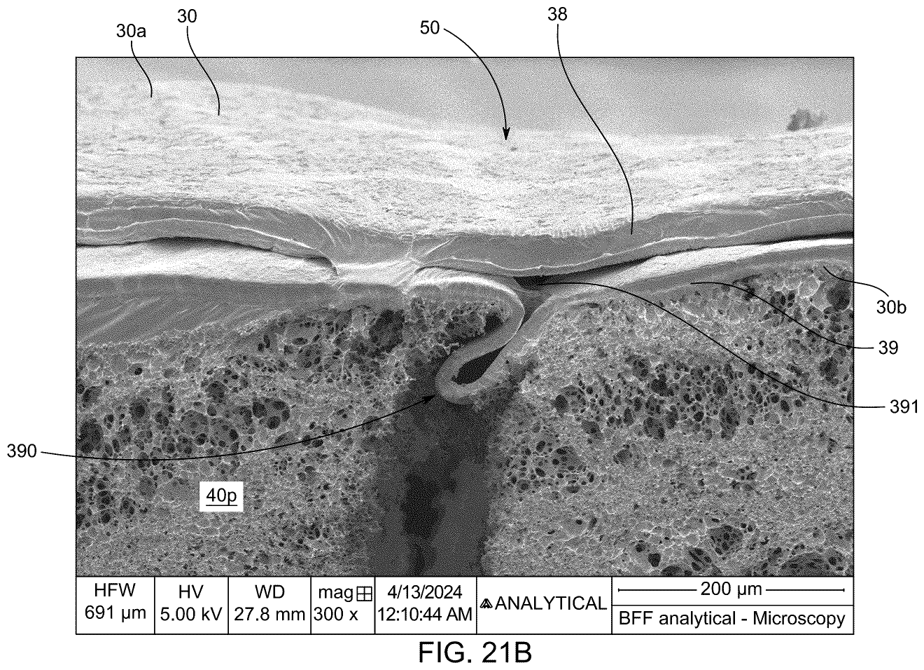

In some configurations, a portion of the backsheet located in the lines of deformation 50 described above may further comprise one or more macro-wrinkles. In particular, it was found that after activation, a portion of the constricting layer 39 located in the lines of deformation 50 include one or more macro-wrinkles 390. FIGS. 20A and 21A are SEM photomicrograph taken at 15× and 50× magnification, respectively, showing a tilted cross-sectional view of an absorbent article as described herein after activation (garment-facing surface 30a of the backsheet 30 facing the viewer) showing a plurality of lines of deformation 50. In some configurations, after activation, the viewable outer surface of the backsheet has plurality of intersecting stripes that indicate zones in the backsheet in which there was a particular range of stretching and/or deformation during the activation process. The absorbent article in FIGS. 20A and 20B comprises a backsheet 30 comprising an elastic layer 38 positioned adjacent to the pieces of absorbent core material 40p and a constricting layer 39 on the garment facing surface of the elastic layer 38. In this configuration, the constricting layer 39 forms the garment facing surface 30a of the backsheet 30. FIGS. 20A and 20B show that after activation, the constricting layer 39 forms one or more macro-wrinkles 390. In the configuration shown in FIGS. 20A and 20B, macro-wrinkles 390 extend from the garment-facing surface 30a in a direction away from the wearer-facing surface 30b such that a gap 391 forms between the wearer-facing surface 30b and the peak of a hill of macro-wrinkle 390. As shown in FIGS. 20A and 20B, macro-wrinkle 390 may be a positive macro-wrinkle and extend in a direction away from the surface of the backsheet. Without being limited by theory, it is believed that during activation constricting layer 39 plastically deforms while the elastic layer stretches and then contracts, creating macro-wrinkle 390. Gap 391 forms between constricting layer 39 and elastic layer 38. The absorbent article in FIGS. 21A and 21B comprises a backsheet 30 comprising a constricting layer 39 positioned adjacent to the pieces of absorbent core material 40p and an elastic layer on the garment facing surface of the constricting layer 39. In this configuration, the elastic layer 38 forms the garment facing surface 30a of the backsheet 30. FIGS. 21A and 21B show that after activation, the constricting layer 39 forms one or more macro-wrinkles 390. In the configuration shown in FIGS. 21A and 21B, macro-wrinkles 390 extend from the garment-facing surface 30a in a direction towards the wearer-facing surface 30b such that gap 391 forms between the garment facing surface 30a and the peak of a hill of macro-wrinkle 390. As shown in FIG. 21B, macro-wrinkle 390 may be a negative macro-wrinkle and extend into the gap between neighboring pieces of absorbent material 40p. As used herein, the term “macro-wrinkle” refers to a fold, ridge or crease that has a height of about 80 microns or more.

Panty fastening adhesive applied to the garment-facing surface of the backsheet may flow over the hills and into the furrows of the macro-wrinkles in the lines of deformation. Accordingly, panty fastening adhesive may be disposed in the furrows of the macro-wrinkles in the lines of deformation and may allow for more contact surface area between the backsheet and the adhesive, leading to a stronger bond between the backsheet and the undergarment.

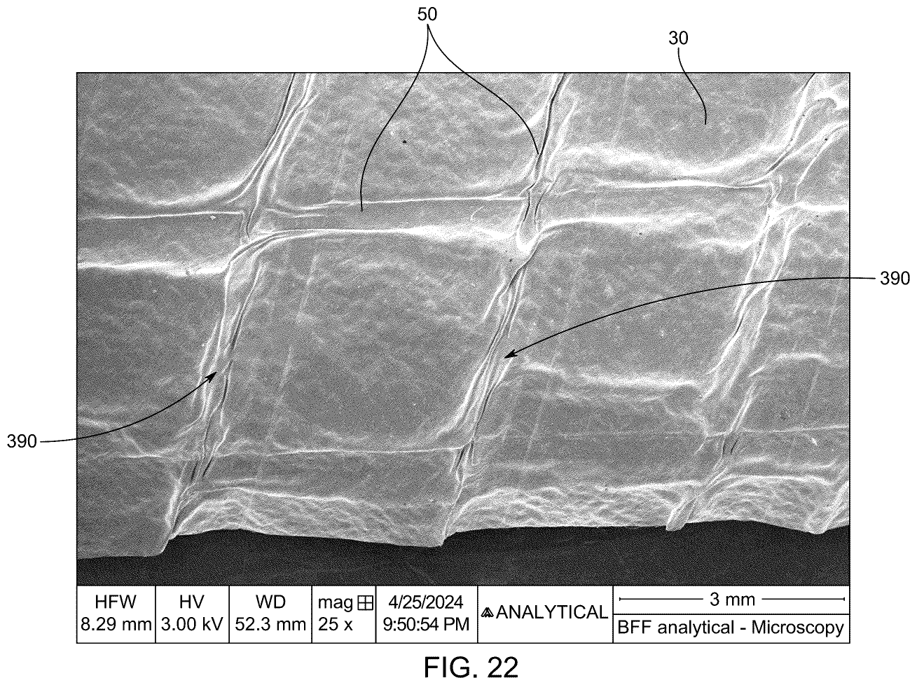

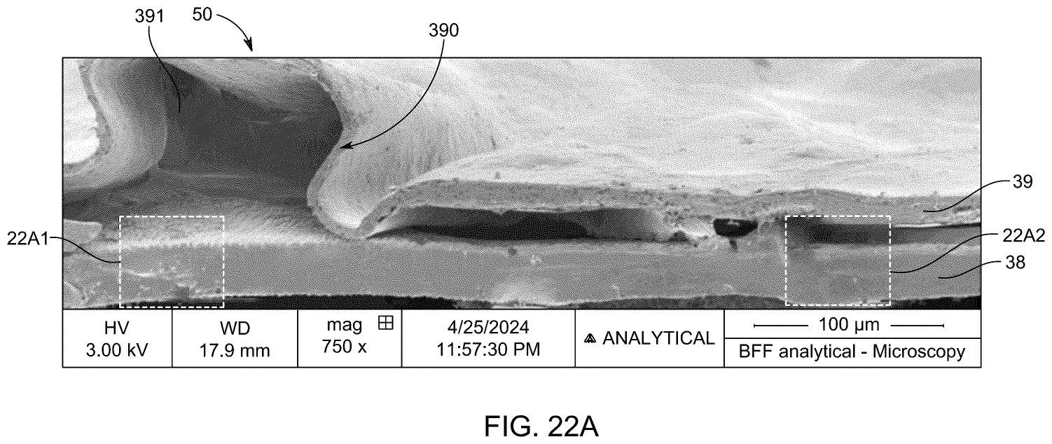

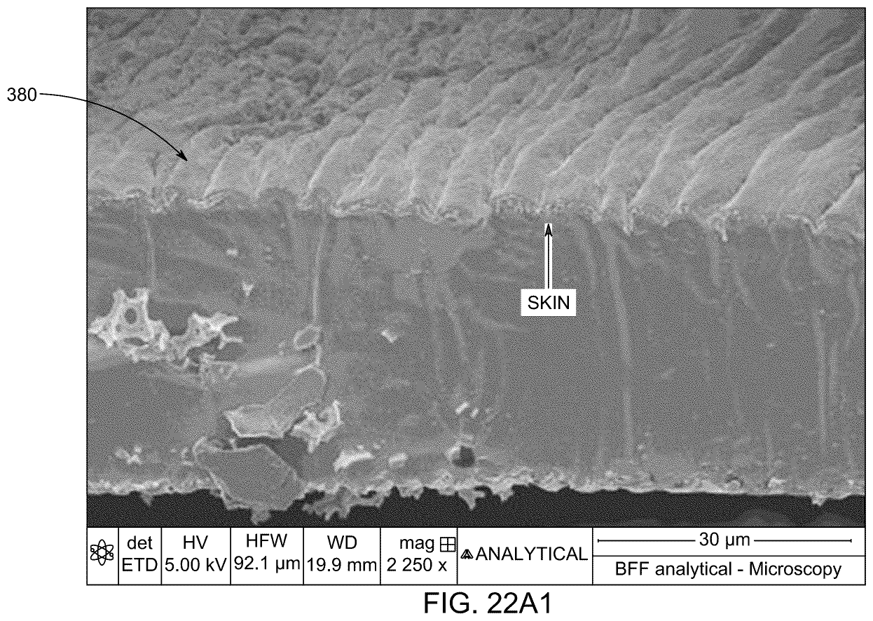

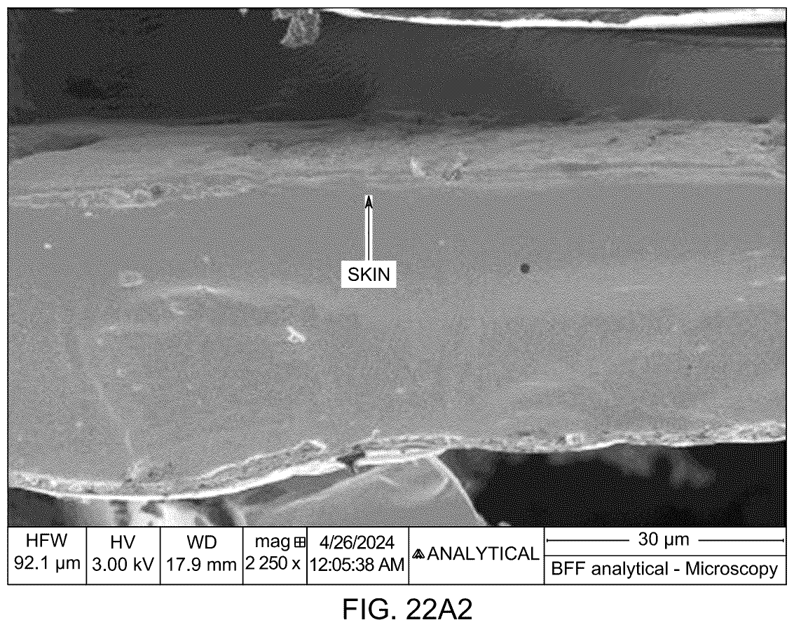

In some configurations, as shown in FIGS. 22-22A2, a portion of the backsheet located in the lines of deformation 50 described above may comprise a plurality of micro-wrinkles 380 and one or more macro-wrinkles 390. FIG. 22 is a SEM photomicrograph taken at 25× magnification showing a tilted top view of an absorbent article described herein after activation, specifically showing a plurality of lines of deformation 50. FIG. 22A is a magnified (750×) SEM photomicrograph cross-sectional view of the absorbent article of FIG. 21A showing a portion of constricting layer 39 located in line of deformation 50 comprises macro-wrinkle 390. FIG. 22A1 is an SEM photomicrograph taken at 2250× magnification showing the portion of FIG. 22A shown in square 22A1. As shown in FIG. 22A1, the skin of the elastic layer located in the line of deformation 50 comprises a plurality of micro-wrinkles 380. FIG. 22A2 is an SEM photomicrograph taken at 2250× magnification showing the portion of FIG. 22A shown in square 22A2. FIG. 22A2 shows that the skin of the elastic layer does not have micro-wrinkles in areas outside of the line of deformation.

The absorbent article described herein may exhibit a minimum MD Full Product Force Threshold at 5 mm extension of about 2.0 N, or about 3.0 N, or about 4.0 N. In some configurations, the absorbent article may exhibit an MD Full Product Force Threshold at 5 mm extension of from about 2.0 N to about 6.0 N. The absorbent article may exhibit a minimum MD Full Product Force Threshold at 10 mm extension of about 7.0 N, or about 8.0 N, or about 9.0 N. In some configurations, the absorbent article may exhibit an MD Full Product Force Threshold at 10 mm extension of from about 7.0 N to about 13.0 N. The absorbent article may exhibit a minimum MD Full Product Force Threshold at 15 mm extension of about 10.0 N, or about 12.0 N, or 15.0 N. In some configurations, the absorbent article may exhibit an MD Full Product Force Threshold at 15 mm extension of from about 10.0 N to about 18.0 N. The absorbent article may exhibit a minimum MD Full Product Force Threshold at 20 mm extension of about 12.0 N, or about 14.0 N, or about 18.0 N. In some configurations, the absorbent article may exhibit an MD Full Product Force Threshold at 20 mm extension of from about 12.0 N to about 21.0 N. MD Full Product Force Threshold can be measured according to the Simple Tensile Method.

The absorbent article may exhibit a minimum OD Full Product Force Threshold at 5 mm extension of about 1.0 N, or about 2.0 N. In some configurations, the absorbent article may exhibit an OD Full Product Force Threshold at 5 mm extension of from about 1.0 N to about 3.0 N. The absorbent article may exhibit a minimum OD Full Product Force Threshold at 10 mm extension of about 2.0 N, or about 4.0 N. In some configurations, the absorbent article may exhibit an OD Full Product Force Threshold at 10 mm extension of from about 2.0 N to about 7.0 N. The absorbent article may exhibit a minimum OD Full Product Force Threshold at 15 mm extension of about 4.0 N, or about 6.0 N. In some configurations, the absorbent article may exhibit an OD Full Product Force Threshold at 15 mm extension of from about 4.0 N to about 10.0 N. The absorbent article may exhibit a minimum OD Full Product Force Threshold at 20 mm extension of about 6.0 N, or about 8.0 N. In some configurations, the absorbent article may exhibit an OD Full Product Force Threshold at 20 mm extension of from about 6.0 N to about 13.0 N. OD Full Product Force Threshold can be measured according to the Simple Tensile Method.

Without being limited by theory, it is believed that an absorbent article having an MD Full Product Force Threshold and/or an OD Full Product Force Threshold as described herein can help to prevent the absorbent article from yielding at low forces below the peak peel force from an undergarment. Yielding of the absorbent article (the force at which the article begins to yield as measured by the 0.2% offset method on the 200% tensile curve of a sample cut from the article) can compromise the integrity of the article and can cause failures such as backsheet tearing and/or detachment of the discreet cells of absorbent material from the backsheet and/or the topsheet at the adhesively adhered surfaces.

In some configurations, the absorbent article can have a Full Product MD Yield Force of greater than 9.0 N as measured according to the Simple Tensile Method. Without being limited by theory, it is believed that by having a Full Product MD Yield Force of greater than 9.0 N, the article be less likely to be damaged at peel forces that exceed the point at which the article begins to yield.

In some configurations, the absorbent article can have a Full Product MD Modulus of greater than 0.42 N/% as measured according to Simple Tensile Method. In some configurations, the absorbent article can have an OD Full Product Modulus of greater than or 0.17 N/% as measured according to the Simple Tensile Method.

### Examples/Data

The following data and examples, including comparative examples, are provided to help illustrate absorbent articles described herein. The exemplified structures are given solely for the purpose of illustration and are not to be construed as limitations of the present disclosure, as many variations thereof are possible without departing from the spirit and scope of the invention.

### 2 Cycle Hysteresis (% Set) Test

2 Cycle Hysteresis is performed on materials, as described below, to assess the ability of the material to stretch and recover. The test is performed according to the Hysteresis Method described herein. The results are shown in Table 1.

Film A (Comparative Example): 19 gsm polyethylene film available from RKW Group (Mannheim, Germany).

Film B: 30 gsm MD5 film available from Exten SA (Mendrisio, Switzerland).

Film C: Laminate formed from combining Film A and Film B with 9 gsm of H2031 adhesive available from Bostik (Wauwatosa, WI).

Film D: 56 gsm CEX-868 film available from Fitesa (Terre Haute, IN).

Film E: 45 gsm CEX-868 film available from Fitesa (Terre Haute, IN).

TABLE 1

| | | | | | | Ratio of 1^{st Cycle} | % Energy | |
|---|---|---|---|---|---|---|---|---|
| | | Film | 1^{st} Cycle | 1^{st} Cycle | 1^{st} Cycle | Force at 100% | Recovered | |
| | MD % Set | MD | MD Force | MD Force | MD Force | Extension to | at 50% | |
| | at 100% | Yield | at 20% | at 50% | at 100% | 1^{st} Cycle | Elongation | |
| | Elongation | Force | Elongation | Elongation | Elongation | Force at 20% | (%) | |
| Film | (%) | (N) | (N) | (N) | (N) | Extension | MD | OD |
| A | 38 | 5.9 | 8.8 | 10.2 | 10.9 | 1.24 | 25 | 24 |
| B | 7 | 1.2 | 1.9 | 2.5 | 2.8 | 1.51 | 61 | 64 |
| C | 16 | 6.9 | 9.8 | 12.1 | 13.2 | 1.34 | 31 | 31 |
| D | 32 | 6.6 | 7.5 | 7.8 | 8.1 | 1.08 | — | — |
| E | 29 | 8.4 | 9.1 | 9.3 | 9.4 | 1.03 | — | — |

It is believed that materials that are particularly suitable for use as a backsheet have a relatively low MD % Set at 100% Elongation (preferably about 35% or less, more preferably below about 30%, and most preferably below about 20%), thus allowing the backsheet to cause the gap between adjacent pieces of absorbent material to become smaller following activation and to help improve fluid handling performance. In addition, it is believed that backsheet materials should have strength and yield in tensile that survives forces during stretching in-use and forces during removal from an undergarment. In a Simple Tensile Method, materials that are particularly suitable for use as a backsheet have a yield force that is higher than typical peak peel forces that occur during removal of the pad from an undergarment.

Another metric for assuring that the backsheet strength is sufficient is to measure the force at a range of extensions that the backsheet may elongate to during usage of the absorbent article. Particularly suitable backsheet materials will have a 1^{st} Cycle MD Force at 20% Elongation of greater than 1.0 N, more preferably above 2.0 N, and most preferably above 5.0 N.

Finally, it is believed that materials that are particularly suitable for use as a backsheet have a Ratio of the 1^{st} Cycle Force at 100% Extension to the 1^{st} Cycle Force at 20% Extension of about 1.0. Without being limited by theory, it is believed that a ratio of about 1.0 indicates that the film is ductility deforming throughout the range of stretch that it would undergo during incremental stretching. This means that the film provides a robust force threshold at or above the preferred 1^{st} cycle load at 20% elongation through 100% elongation. This range should be between 1.6 to 1.0, preferably between 1.4 to 1.0, and most preferably from 1.2 to 1.0.

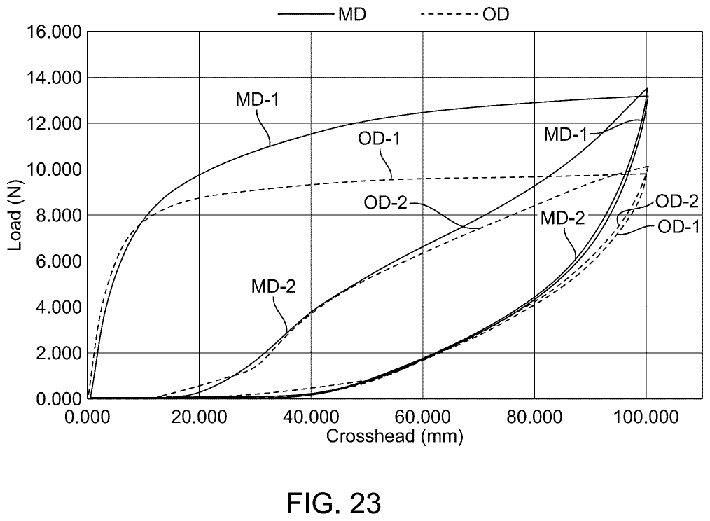

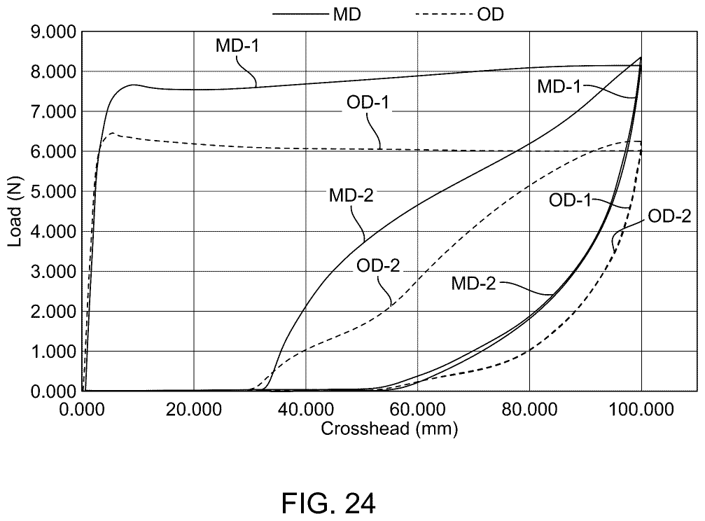

It was found that Films B-E are suitable materials for a backsheet as described herein. Film B is an elastic film comprising an elastic layer having a skin on each surface. Film Cis an elastic film laminate comprising an elastic layer (having skins on each surface) and a constricting layer. Films D and E are elastic films comprising an elastic layer and a constricting layer which have been co-extruded. Films B-E have an MD % Set at 100% Elongation of between 7% and 32%. The 2 cycle hysteresis curves in FIGS. 23-24 depict the elastic behavior exhibited by Film C and D, respectively. The curves MD-1 and OD-1 represent the response to an applied and released elongation (in the MD and OD directions, respectively) during the first cycle and curves MD-2 and OD-2 represent the response to applied and released elongation (in the MD and OD directions, respectively) during the second cycle.

While Film B has a relatively low MD % Set at 100% Elongation of 7%, which helps to form a desired gap size between the pieces of absorbent material, it may be preferable to use a relatively less aggressive panty fastening adhesive configuration with this backsheet material such that the article can easily be removed from an undergarment by the consumer after use without backsheet tearing.

Without being limited by theory, it is believed that absorbent articles comprising a backsheet having an MD % Set at 100% Elongation of 10% or less may be able to readily move with the undergarment during wear and may require less force to adhere the article to the undergarment. This may be accomplished, for example, by reducing the basis weight of panty fastening adhesive and/or by using patterned panty fastening adhesive applications.

### Force Threshold Test

Samples 1-4 are feminine hygiene pads in accordance with the present disclosure having a 25 gsm spunbond bicomponent (PE/PP) topsheet, HIPE foam core, and a backsheet comprising the material according to Table 2 below. The topsheet and the backsheet are joined to the HIPE foam core using 9 gsm spiral adhesive (H2301 available from Bostik). The feminine hygiene pads are activated as described herein and shown in FIG. 7 to fracture the HIPE foam into discrete pieces. The pads are tested to assess the impact of the backsheet material on the MD and Orthogonal (OD) Force Threshold at various extensions. Sample 1 is a comparative example. Films A-D are described above. The test is performed according to the Simple Tensile Method as described herein. The results are shown in Table 2.

TABLE 2

| | | MD Full Product Force Threshold | | | | OD Full Product Force Threshold | | | |
|---|---|---|---|---|---|---|---|---|---|
| | | (N) | | | | (N) | | | |
| | Backsheet | 5 mm | 10 mm | 15 mm | 20 mm | 5 mm | 10 mm | 15 mm | 20 mm |
| Sample | Material | Ext. | Ext. | Ext. | Ext. | Ext. | Ext. | Ext. | Ext. |
| 1 | Film A | 6.3 | 12.4 | 15.9 | 18.0 | 3.7 | 7.5 | 9.9 | 11.2 |
| 2 | Film B | 2.7 | 8.0 | 11.0 | 12.6 | 1.4 | 2.6 | 4.8 | 6.4 |
| 3 | Film C | 5.4 | 12.5 | 17.5 | 20.5 | 3.0 | 6.4 | 9.7 | 11.9 |
| 4 | Film D | 5.1 | 11.4 | 15.5 | 18.2 | 2.7 | 5.5 | 9.0 | 11.4 |

Table 2 shows the MD and OD Full Product Force Threshold for Samples 1-4 over different extensions. Without being limited by theory, it is believed that absorbent articles having an MD and/or OD Force Threshold at 15 mm extension that is greater than the peak peel force can create a force wall such that the backsheet will not continue to stretch uncontrolled during removal, thus reducing the risk of backsheet tearing. Testing with consumers has shown that consumers can exert a large amount of force to remove an absorbent article from their undergarment. Testing has shown that the threshold forces of the absorbent article described herein can be important preventing the article from overly extending in response to the removal forces. For absorbent articles comprising an elastic backsheet having a MD and/or OD % Energy Recovered at 50% Elongation of up to 35%, it is preferable that the OD Full Product Force Threshold at 15 mm Extension be greater than 7.2 N in order to reduce the risk of backsheet tearing and/or loss of core integrity during removal of the article from an undergarment. Absorbent articles comprising backsheets having an MD and/or OD % Energy Recovered at 50% Elongation of greater than 35% may be able to move more readily with the undergarment during wear and may require less force to adhere the article to the undergarment. For absorbent articles comprising a backsheet having an MD and/or OD % Energy Recovered at 50% Elongation of greater than 35%, it may be preferable that the OD Full Product Force Threshold at 15 mm Extension be greater than 3.6 N but less than 7.2 N in order to reduce the risk of backsheet tearing and/or loss of core integrity during removal of the article from an undergarment. This may be accomplished, for example, by reducing the basis weight of panty fastening adhesive and/or by using patterned panty fastening adhesive applications.

### Gap Size Test

Samples 5-9 are feminine hygiene pads in accordance with the present disclosure having a 25 gsm spunbond bicomponent (PE/PP) topsheet, HIPE foam core, and a backsheet comprising the material according to Table 3 below. The topsheet and the backsheet are joined to the HIPE foam core using 9 gsm spiral adhesive (H2301 available from Bostik). The feminine hygiene pads are activated as described herein and shown in FIG. 7 to fracture the HIPE foam into discrete pieces. The pads are tested to assess the gap size in a relaxed and stretched state. Samples 5 is a comparative example. The test is performed according to the SEM Imaging Method and the Stretched Gap Measurement Method as described herein. The results are shown in Table 3.

TABLE 3

| | | SEM | Stretched Gap |
|---|---|---|---|
| | | Imaging Method | Measurement Method |
| | Backsheet | Relaxed Gap | Stretched Gap |
| Sample | Material | (microns) | (microns) |
| 5 | Film A | 296.1 | 800 |
| (Comp. Ex.) | | | |
| 6 | Film B | 100.1 | 3000 |
| 7 | Film C¹ | 84.4 | 800 |
| 8 | Film C² | 96.6 | 800 |
| 9 | Film D | 86.5 | 1000 |

¹The Film C laminate is configured in the feminine hygiene pad with the Film B layer on the wearer facing surface of the backsheet.
²The Film C laminate is configured in the feminine hygiene pad with the Film B layer on the garment facing surface of the backsheet.

Table 3 shows the Relaxed Gap (at 0 g force) and the Stretched Gap (at 768 g force) between the pieces of HIPE foam. Sample 5, which contains Film A (a non-elastic film) as a backsheet and has a Relaxed Gap size of 296.1 microns. Samples 6-9, which contain elastic Films B-D, respectively, have a Relaxed Gap size of from 86.5 microns to 100.1 microns. Samples 6-9 have narrow relaxed gap sizes that can enable low rewet (as demonstrated in the Fluid Acquisition and Rewet Method) by re-establishing fluid distribution between the pieces of HIPE foam. It was also found that pads made with Film B as the backsheet (which does not contain a constricting layer) can elongate and creep causing the gap between the cells of HIPE to elongate to relatively wide gaps and lead to delamination of the cells from the topsheet and/or backsheet. Without being limited by theory, it is believed that Film B may be suitable as a backsheet in the absorbent articles described herein when the OD Full Product Force Threshold of the article is limited to a range of greater than 3.6 N and less than 7.2 N.

### Fluid Management Test

Consumers may prefer absorbent articles that can deliver a dry wearing experience. Feminine hygiene pads are prepared to assess the ability of the pad to effectively drain fluid. Samples 10-14 are feminine hygiene pads in accordance with the present disclosure having a 25 gsm spunbond bicomponent (PE/PP) topsheet, HIPE foam core, and a backsheet comprising the material according to Table 4 below. The topsheet and the backsheet are joined to the HIPE foam core using 9 gsm spiral adhesive (H2301 available from Bostik). Samples 10-14 are activated as described herein and shown in FIG. 7 to fracture the HIPE foam into discrete pieces. Sample 10 is a control and is not activated. Sample 11 is a comparative example. The test is performed according to the Acquisition Time and Rewet Method. The results shown in Table 4.

TABLE 4

| | | | Surface | Interfacial | |
|---|---|---|---|---|---|
| | | | Free Fluid | Free Fluid | SFF + |
| | Backsheet | Gap Size | (SFF) | (IFF) | Rewet |
| Samples | Material | microns | (mg) | (mg) | (mg) |
| 10 | Film A | 0 | 16 | 7 | 42 |
| (Control) | | | | | |
| 11 | Film A | 296.1 | 189 | 22 | 871 |
| (Comp. Ex) | | | | | |
| 12 | Film B | 100.1 | 18 | 7 | 55 |
| 13 | Film C ³ | 84.4 | 17 | 10 | 45 |
| 14 | Film E | 84.4 | 12 | 6 | 39 |

³ The Film C laminate is configured in the feminine hygiene pad with the Film B layer on the wearer facing surface of the backsheet.

Table 4 depicts fluid handling performance of feminine hygiene pads made with a non-elastic backsheet that is not activated (Sample 10), a non-elastic backsheet that is activated (Sample 11), and elastic backsheets that are activated (Samples 12-14). The table shows the trade-off that occurs when we create large gaps between the HIPE foam cells (>280 micron) and how rewet values and surface free fluid (SFF) can increase to 10× to 20× higher than control products. Pads containing elastic backsheets have smaller gaps (about 100 micron or less) that can re-establish fluid distribution between HIPE foam cells and lower Rewet, SFF, and IFF to levels where the consumer will have better perception that the pad is drier.

### Test/Measurement Methods

### Conformability Force Measurement Method

Measurements made using this Conformability Force Measurement Method reflect the extent to which a composite web material (i.e., combination, assembly or laminate of web materials) will resist bending and stretching around a non-ruled surface. A composite web material that is bidirectionally extensible will more readily bend and stretch around a non-ruled surface, than a comparable composite that is not bidirectionally extensible. In this method, the non-ruled surface is a spherical ball that is 25.4 mm in diameter.

The Conformability Force Measurement Method is performed on a constant rate of extension tensile tester (a suitable instrument is MTS Insight tensile tester operated under TestSuite software, MTS Systems Corp, Eden Prairie, MN, or equivalent) using custom fixtures and an appropriate capacity load cell. All testing is performed in a laboratory controlled at 23° C.±2° C. and 50%±2% relative humidity.

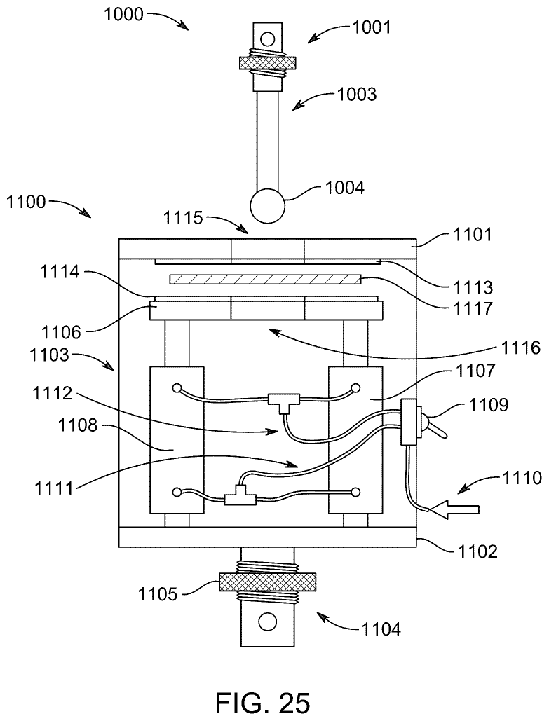

Referring to FIG. 25, the bottom fixture 1100 is a pneumatic clamping system used to secure the specimen 1117 horizontally for testing. The fixture is built of a box 1103 made of 6.4 mm Plexiglass with a top 1101 and a bottom 1102 both made of 9.5 mm thick aluminum plates. The bottom 1102 plate is attached to the bottom mount of the tensile tester via an adapter 1104 and locking collar 1105 used to secure the fixture orthogonal to the mount of the tensile tester. Interior to the box a movable plate 1106 made of 9.5 mm thick aluminum is attached to two pneumatic cylinders 1107 and 1108 used to raise and lower the test specimen 1117. A rubber gasket 1113 is affixed on the bottom side of the top plate 1101 with a matching rubber gasket 1114 affixed to the top of the movable plate 1106. A circular 50.1 mm diameter, vertically oriented orifice 1115 passes through the longitudinal and lateral center of the top plate 1101 and gasket 1113. A corresponding circular 50.1 mm diameter orifice 1116 is vertically aligned with orifice 1115 and passes through the movable plate 1106 and gasket 1114. Pressurized air 1110 is provided to a switch 1109 which is fluidly connected via tubing 1111, 1112 to the cylinders 1107, 1108 and is used to raise and lower the movable plate 1106. The air pressure is sufficient to hold the sample securely without slippage for testing.

The upper fixture includes a cylindrical plunger 1003 terminating in a ball 1004 with a diameter of 25.4 mm. The plunger has an adapter 1001 compatible with the mount on the load cell capable of securing the plunger orthogonal to the top plate 1101 of the bottom fixture. When the fixtures are assembled with the tester, the ball 1004 is vertically centered over orifices 1115 and 1116, and the center of the ball travels along a vertical path coincident with the vertical axes of the orifices. The gage length is set at 10 mm between the bottom surface of the ball 1004 and the bottom surface of gasket 1113.

Program the instrument for compression mode. Lower the crosshead at a rate of 500 mm/min for the specified engagement distance and return the crosshead to its original position. Record data from the force and distance channels at a rate of 100 Hz during the downward stroke. Run a measurement cycle on each sample for three specified crosshead distances: 15 mm (i.e., 5 mm engagement) 20 mm, (i.e., 10 mm engagement) and 25 mm (i.e., 15 mm engagement).

Test sample products are conditioned in a laboratory controlled at 23° C.±2° C. and 50%±2% relative humidity. Place the sample on a flat bench, top sheet facing upward. Identify the longitudinal axis of the sample. Measure down 45 mm from the top edge of the absorbent structure along the longitudinal axis and mark with a dot. This is the center of the measurement site. Remove release paper (or adhesive coversheet) (if present) from the sample. Center the marked dot within the opening in the lower plate. Prior to securing the sample between the gaskets 1113, 1114, it should be gently pulled taut with approximately equal tension applied along two perpendicular directions, only to an extent sufficient eliminate sagging over the opening 1116. After pulling the sample taut, activate the switch 1109 to admit air into the cylinders 1107, 1108 and thereby raise the movable plate 1106, to secure the sample between the gaskets 1113, 1114. Zero the force and crosshead channels and start the program. After the test, lower the movable plate and remove the sample prior to analyzing the next sample. Testing is performed on seven (7) replicate samples at each of the 3 crosshead distances.

Construct a graph of force (N) verses displacement (mm) for each test run. Read the peak force (N) at from the graph and record to the nearest 0.01 N. Calculate the Conformability Force as the greatest slope of the curve utilizing a line segment that is at least 20% of the maximum force and record to the nearest 0.1 N/m. Report Conformability Force as the average of seven replicate specimens to the nearest 1 N/m, at a displacement of the sample to 5 mm from its starting position (i.e., after the ball first contacts the sample it moves downward to a maximum of 5 mm).

### Capillary Work Potential Via Pore Volume Distribution

Pore Volume Distribution determines the estimated porosity of the effective pores within a porous sample by measuring the fluid movement into and out of said sample as stepped, controlled differential pressure is applied to the sample in a sample chamber. The incremental and cumulative quantity of fluid that is thereby absorbed/drained by the porous sample at each pressure is then determined. In turn, work done by the porous sample normalized by the area of said sample is calculated as Capillary Work Potential.

### Method Principle

For uniform cylindrical pores, the radius of a pore is related to the differential pressure required to fill or empty the pore by the following equation:

$$Differential Pressure = [ 2 γ cos Θ ) ] / r$$

$$where γ = liquid surface tension , Θ = contact angle , and r = pore radius .$$

Pores contained in natural and manufactured porous materials are often thought of in terms such as voids, holes or conduits, and these pores are generally not perfectly cylindrical nor all uniform. One can nonetheless use the above equation to relate differential pressure to an effective pore radius, and by monitoring liquid movement into or out of the material as a function of differential pressure, characterize the effective pore radius distribution in said porous material. (Because nonuniform pores are approximated as uniform by the use of an effective pore radius, this general methodology may not produce results precisely in agreement with measurements of void dimensions obtained by other methods such as microscopy.)

The Pore Volume Distribution method uses the above principle and is reduced to practice using the apparatus and approach described in “Liquid Porosimetry: New Methodologies and Applications” by B. Miller and I. Tyomkin published in The Journal of Colloid and Interface Science (1994), volume 162, pages 163-170, incorporated herein by reference. This method relies on measuring the increment of liquid volume that enters or leaves a porous sample as the differential air pressure is changed between ambient (“lab”) air pressure and a slightly elevated air pressure (positive differential pressure) surrounding the sample in a sample test chamber. The sample is introduced to the sample chamber dry, and the sample chamber is controlled at a positive differential pressure (relative to the lab) sufficient to prevent fluid uptake into the sample after the fluid bridge is opened. After opening the fluid bridge, the differential air pressure is decreased in steps to 0, and in this process subpopulations of pores within the sample acquire liquid according to their effective pore radius. After reaching a minimal differential pressure at which the mass of fluid within the sample is at a maximum, differential pressure is increased stepwise again toward the starting pressure, and the liquid is drained from the sample. The absorption portion of the stepped sequence begins at the maximum differential pressure (smallest corresponding effective pore radius) and ends at the minimum differential pressure (largest corresponding effective pore radius). The drainage portion of the sequence begins at the minimum pressure differential and ends at the maximum pressure differential. After correcting for any fluid movement for each particular pressure step measured on the chamber while empty for the entire absorption/drainage sequence, the fluid uptake by the sample (mg) as well as cumulative volume (mm³/mg) at each differential pressure is determined in this method.

### Sample Conditioning and Sample Preparation

The Pore Volume Distribution method is conducted on samples that have been conditioned for at least 2 hours in a room maintained at a temperature of 23° C.±2.0° C. and a relative humidity of 50%±2%, and all tests are conducted under the same environmental conditions in such conditioned room. Any damaged product or sample that has defects such as wrinkles, tears, holes, and similar are not tested. A sample conditioned as described herein is considered dry for purposes of this invention. Determine which side of the sample is intended to face the wearer in use, then cut it to 55 mm long by 55 mm wide. Measure the mass of the sample and record to the nearest 0.1 mg. Three samples are measured for any given material being tested, and the results from those three replicates are averaged to give the final reported values.

### Apparatus

Apparatus suitable for this method is described in “Liquid Porosimetry: New Methodology and Applications” by B. Miller and I. Tyomkin published in The Journal of Colloid and Interface Science (1994), volume 162, pages 163-170. Further, any pressure control scheme capable of controlling the sample chamber pressure between 0 mm H₂0 and 1098 mm H₂0 differential pressure may be used in place of the pressure-control subsystem described in this reference. One example of a suitable overall instrumentation and software is the TRI/Autoporosimeter (Textile Research Institute (TRI)/Princeton Inc. of Princeton, NJ, USA). The TRI/Autoporosimeter is an automated computer-controlled instrument for measuring pore volume distributions in porous materials (e.g., the volumes of different size pores within the range from 5 μm to 1200 μm effective pore radii). Computer programs such as Automated Instrument Software Releases 2000.1 or 2003.1/2005.1 or 2006.2; or Data Treatment Software Release 2000.1 (available from TRI Princeton Inc.), and spreadsheet programs may be used to capture and analyze the measured data.

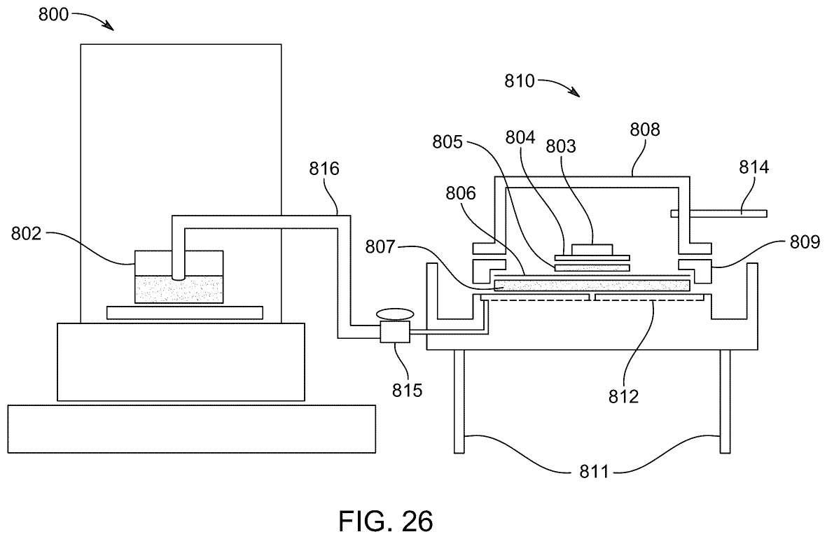

A schematic depiction of suitable equipment is shown in FIG. 26. The equipment consists of a balance 800 with fluid reservoir 802 which is in direct fluid communication with the sample 805 which resides in a sealed, air-pressurized sample chamber 810. The fluid communication between the reservoir 802 and the sample chamber 810 is controlled by valve 815. A weight 803 placed on top of a Plexiglass plate 804 (55 mm long by 55 mm wide) is used to apply a confining pressure of 0.25 psi on the test sample to ensure good contact between the sample and a fluid saturated membrane 806 throughout the test. The membrane 806 (90 mm diameter, 150 μm thick, 1.2 μm pore size; mixed cellulose ester filter RAWP09024; available from Millipore Corporation of Bedford, MA) is attached to a macro-porous frit 807 (Monel plate with 90 mm diameter, 60 mm thick; available from Mott Corporation, Farmington, CT, or equivalent) as follows. Adhere the membrane 806 to the frit 807 using Krylon® spray paint (Gloss White Spray Paint #1501; available from FilmTools, or equivalent) as the adhesive. Allow the prepared membrane/frit assembly to dry prior to use.

To prepare the equipment for testing, fill the inner base 812 of the sample chamber 810 with test fluid. The test fluid is degassed 0.9% saline solution, prepared by adding 9.0 g of reagent grade NaCl per 1 L of deionized water (liquid density is 1.01 g/cm³, surface tension y to be 72.3±1 mN/m, contact angle cos Θ=0.37). Place the membrane/frit assembly, membrane 806 side up, onto the inner base 812 of the sample chamber 810, and secure it into place with a locking collar 809. Fill the reservoir 802 and connecting tube 816 with test fluid. Open valve 815 and ensure that no air bubbles are trapped within the connecting tube or the pores within the membrane/frit assembly. Using the legs 811 of the sample chamber 810, level the sample chamber and adjust the height of the sample chamber (and/or the amount of fluid in the reservoir 802) as necessary, to bring the top surface of the membrane 806 into the same horizontal plane as the top surface of the fluid in the reservoir 802.

Program the system to progress through a sequence of stepped differential pressures (in mm H₂0) as follows: 1098, 549, 366, 275, 220, 183, 137, 110, 92, 78, 69, 61, 55, 50, 46, 42, 39, 37, 34, 32, 31, 29, 27, 24, 22, 20, 18, 14, 9.2, 6.9, 5.5, 4.6, 5.5, 6.9, 9.2, 14, 18, 20, 22, 24, 27, 29, 31, 32, 34, 37, 39, 42, 46, 50, 55, 61, 69, 78, 92, 110, 137, 183, 220, 275, 366, 549, 1098. These pressures correlate to effective pore radii from 5 μm (1098 mm H₂0) to 1200 μm (4.6 mm H₂0). The criterion for moving from one pressure step to the next is that fluid uptake/drainage to/from the sample, measured at the balance 800, is less than 10 mg/min for 15 seconds.

### Method Procedure

Check the system for leaks and ensure the maximum test pressure can be reached as follows. With liquid valve 815 open, place the top 808 of the sample chamber 810 in place and seal the chamber. Apply sufficient air pressure to the chamber 810 (via connection 814) to achieve a differential pressure of 1098 mm H₂O (5 μm effective pore radius). Close the liquid valve 815 then open the sample chamber. Place the sample 805 (wearer side facing down) directly onto the membrane 806, then place the cover plate 804 and confining weight 803 centered over the sample. Replace the top 808 and reseal the sample chamber 810. Open the liquid valve 815 to allow movement of fluid between the liquid reservoir 802 and the sample and start the test to progress through the pre-specified sequence of differential pressures. The amount of fluid absorbed (or drained) by the sample at each pressure step over the entire sequence is recorded as Uptake to the nearest 0.1 mg.

A separate “blank” measurement is performed by following this same method procedure (same stepped sequence of differential pressures) on an empty sample chamber with no sample 805, cover plate 804 or confining weight 803 present on the membrane/frit assembly. Any fluid movement observed is recorded (mg) at each of the pressure steps. Fluid uptake data for the sample are corrected for any fluid movement associated with the empty sample chamber by subtracting fluid uptake values of this “blank” measurement from corresponding values in the measurement of the sample, and recorded as Blank Corrected Sample Uptake to the nearest 0.1 mg.

### Determination of Capillary Pressure, Cumulative Volume and Capillary Work Potential

The % Saturation of the sample at each of the pressure steps for both the absorption and drainage portions of the test sequence can be calculated by dividing the maximum Blank Corrected Sample Uptake (mg) by the Blank Corrected Sample Uptake (mg), then multiplying by 100. From the data collected across the entire sequence, one of average skill in the art can then determine the % Saturation at any given capillary absorption pressure (CAP) or capillary desorption (drainage) pressure (CDP). The CAP and CDP are reported to the nearest 0.1 mm H₂O for any specified % Saturation.

The cumulative volume is calculated for each of the pressure steps by the following equation:

Cumulative Volume (mm³/mg)=Blank Corrected Sample Uptake (mg)/Fluid Density (g/cm³)/Mass of Sample (mg)

The Capillary Work Potential (CWP) is the work done by the sample normalized by the area of the sample. The trapezoidal rule is used to integrate the ith Pressure as a function of Cumulative Volume over n data points for the absorption and drainage portions of the cycle.

$$CWP [ \frac{m J}{{m}^{2}} ] = \frac{W}{{A}_{w}} = {\sum }_{i = 1}^{n} \frac{1}{2} \frac{{m}_{w} ( C {V}_{i + 1} - C {V}_{i} ) ( {P}_{i} + {P}_{i + 1} )}{{A}_{w}} ( 1 {0}^{3} [ \frac{m J}{J} ] )$$

$$where$$

$${m}_{w} = mass of sample ( mg )$$

$$CV = Cumulative Volume ( {m}^{3} / mg )$$

$$P = Air Pressure ( Pa )$$

$${A}_{w} = Area of sample on one side ( {m}^{2} )$$

Report the CWP, CWPA and CWPD to the nearest 0.1 mJ/m², where CWPA represents the absorption portion of the pressure sequence and CWPD represents the drainage portion of the pressure sequence.

### Caliper Measurement

The caliper, or thickness, of a test sample of a nonwoven web, laminate, foam layer material, or combination thereof, is measured as the distance between a reference platform on which the sample rests and a pressure foot that exerts a specified amount of pressure onto the sample over a specified amount of time. All measurements are performed in a laboratory maintained at 23° C.±2° C. and 50%±2% relative humidity and test samples are conditioned in this environment for at least 2 hours prior to testing.

Caliper is measured with a manually-operated micrometer equipped with a pressure foot capable of exerting a steady pressure of 2.0 kPa±0.01 kPa onto the test sample. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter that is smaller than the test sample and capable of exerting the required pressure. A suitable pressure foot has a diameter of 25.4 mm, however a smaller or larger foot can be used depending on the size of the sample being measured. The test sample is supported by a horizontal flat reference platform that is larger than and parallel to the surface of the pressure foot. The system is calibrated and operated per the manufacturer's instructions.

Obtain a test sample, if necessary by removing it from an absorbent article. When excising the test sample from an absorbent article, use care to not impart any contamination or dimensional deformation to the test sample. The test sample is obtained from an area free of folds or wrinkles, and it must be larger than the pressure foot.

To measure caliper, first zero the micrometer against the horizontal flat reference platform. Place the test sample on the platform with the test location centered below the pressure foot. Gently lower the pressure foot with a descent rate of 3.0 mm+1.0 mm per second until the full pressure is exerted onto the test sample. Wait 5 seconds and then record the caliper of the test sample to the nearest 0.01 mm. In like fashion, repeat for a total of five replicate test samples. Calculate the arithmetic mean for all caliper measurements and report as Caliper to the nearest 0.01 mm.

### High Speed Tensile Test

The High Speed Tensile Test is used to measure the Tensile Strength of a material sample at a relatively high strain rate. The method uses a suitable tensile tester such as an MTS 810, available from MTS Systems Corp., Eden Prairie, Minnesota, or equivalent, equipped with a servo-hydraulic actuator capable of facilitating speeds exceeding 1 m/s after 5 mm of crosshead displacement, and at least approximately 1.5 m/s after 10 mm of crosshead displacement. The tensile tester is fitted with a 50-lb force transducer (part 9712 B50 from Kistler North America, Amherst, New York, or equivalent), and a signal conditioner with a dual mode amplifier (part 5010 from Kistler North America, or equivalent).

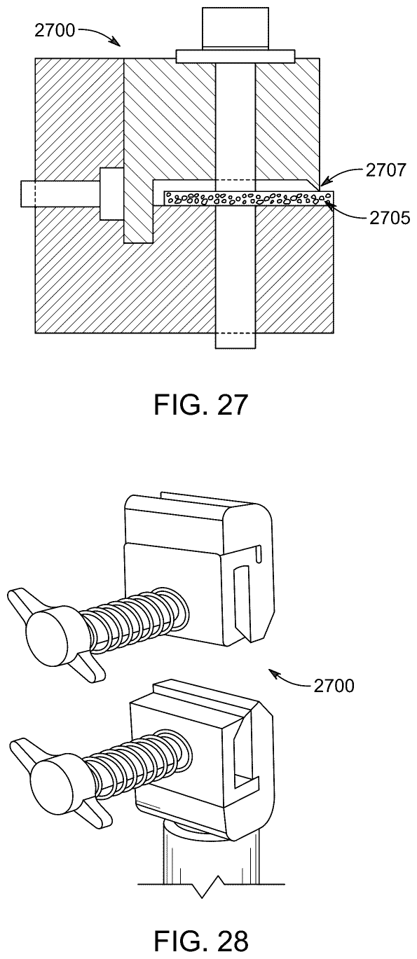

FIG. 27 is a cross-sectional view of a single line contact grip 2700 used in this test. The line grip 2700 is selected to provide a well-defined gauge and avoid undue slippage of the specimen. The specimen is positioned such that it has minimal slack. The apex 2707 of the grip 2700 is ground to give good gauge definition while avoiding damage or cutting of the specimen. A portion of the grip 2700 may be configured to include a material 2705 that reduces the tendency of a specimen to slip. FIG. 28 illustrates a pair of opposing line contact grips 2700 suitable for use in this test. A pair of grips of varying specific design but with equivalent function (i.e., capable of facilitating a well-defined 3-mm gauge length, no undue slippage, and at least as wide as the specimens analyzed) to those described above may be used alternatively.

For a nonwoven sample of interest, five like specimens having dimensions of 50.8 mm wide by 15 mm long are cut. The short dimension of each specimen is parallel to the machine direction of the nonwoven. If the specimens are extracted from finished absorbent article(s), the short dimension of the specimen is oriented parallel to the longitudinal axis of the absorbent article. The line contact grips are moved to a grip separation of 3.0 millimeters (i.e., the distance between the lines of contact between specimen and grip surface). The specimen is mounted in the line contact grips, and a thin piece of tape to help hold the specimen straight and flat while mounting in grips. (If used, tape must remain behind the lines of gripping so that it does not interfere with the specimen gauge during the test.) The line contact grips are moved closer together to put as much slack as possible into the film specimen without the line contact grips interfering with one another. Actuator movement is selected such that the specimen experiences relative grip speed of approximately 1 m/s at an engineering strain of 1 and 1.5 m/s at an engineering strain of 4. Typically, during testing, one of the line contact grips is kept stationary and the opposing line contact grip is moved, but forms where both line contact grips move are also contemplated herein.

The force and actuator displacement data generated during the test are recorded using a Nicolet Integra Model 10, 4-channel 1 Ms/s, 12-bit digitizer oscilloscope with the data acquisition frequency set at 50 kHz. The resulting data are expressed as force (measured in Newtons) versus engineering strain. The engineering strain (ϵ) is dimensionless and is defined as

$$ϵ = \frac{L - {L}_{0}}{{L}_{0}} = \frac{z}{{L}_{0}}$$

where:
L₀ is the gauge length (i.e., the distance between lines of grip contact when the undeformed specimen is mounted in the grips). (The L₀ in the present example is 3.0 mm.)
L is grip position, the distance between lines of grip contact during the tensile test.
And z is displacement, defined as z=L−L₀.

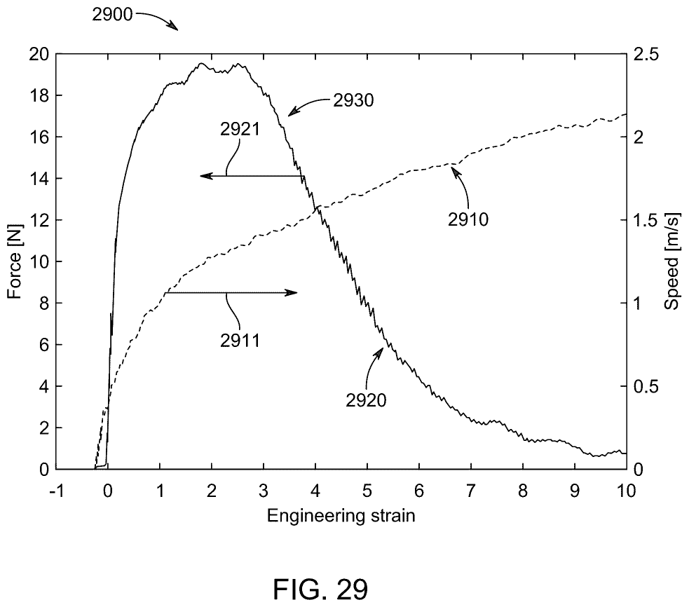

FIG. 29 illustrates a suitable example graph 2900 with two curves 2910 and 2920. The first curve 2910 illustrates a plot of actuator speed (i.e., the relative speed at which one grip is moving away from the other grip) versus engineering strain. The arrow 2911 points to the vertical axis at right used for the plot 2910. The second curve 2920 illustrates a plot of force versus engineering strain and uses the vertical axis at left, as indicated by the arrow 2921. The point of maximum force in the force versus engineering strain plot is identified. Moving toward higher engineering strain, the first point at which the force falls to equal to or less than 90% of the maximum force value is then identified, and the engineering strain at that point is defined as the Extensibility Parameter of the specimen and is recorded to the nearest 0.01. (The region in which this point is found is indicated by arrow 2930.)

The arithmetic mean of the five Extensibility Parameter values determined for each of the five like specimens is reported to the nearest 0.01 as the Extensibility Parameter of the material sample.

### Acquisition Time and Rewet Method

This method describes how to measure gush acquisition time, interfacial free fluid amount as well as low and high pressure rewet values for an absorbent article loaded with new Artificial Menstrual Fluid (nAMF; preparation provided separately herein). A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low and high pressure rewet are measured after the last liquid dose. Surface Free Fluid (SFF) is the amount of fluid that remains in the topsheet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2° C. and 50%±2% relative humidity.

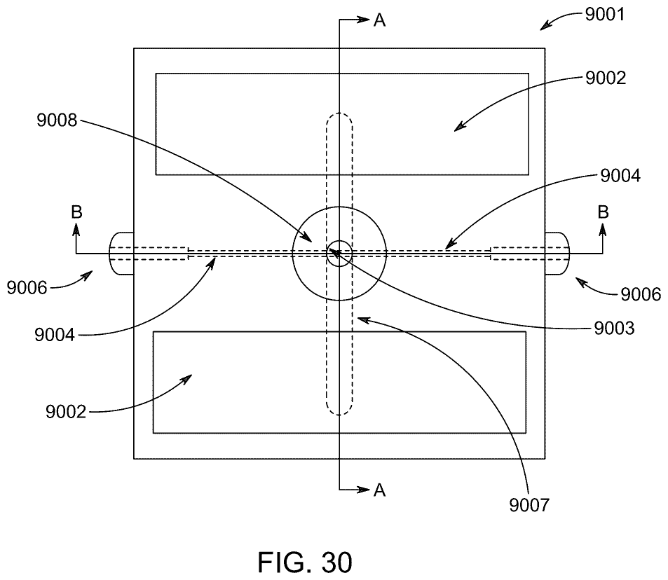

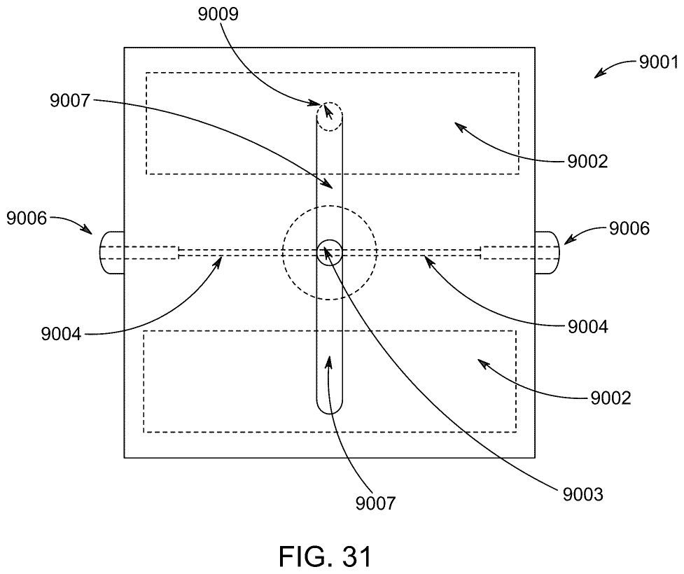

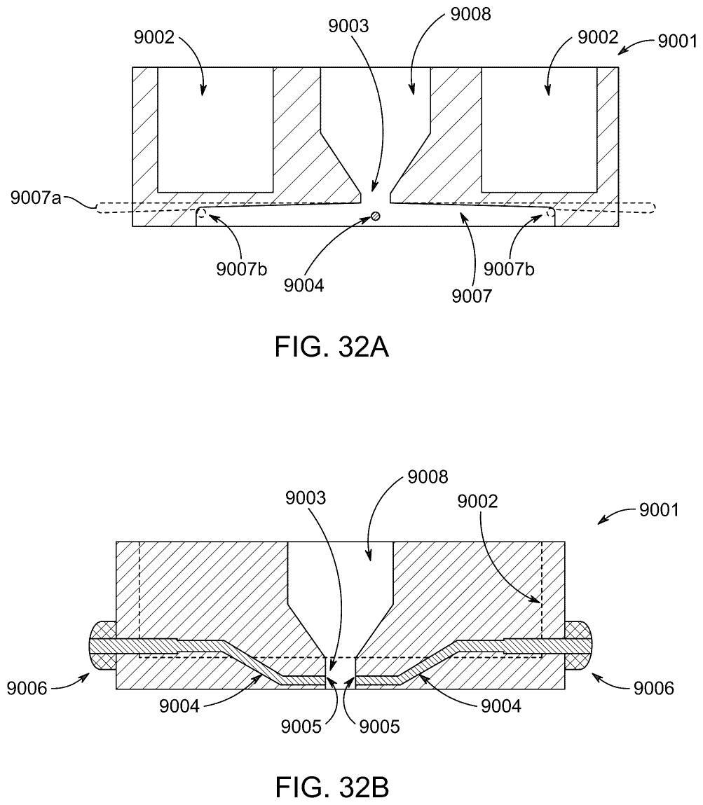

Referring to FIGS. 30-32B, the strikethrough plate 9001 is constructed of Plexiglas, or equivalent, with an overall dimension of 10.2 cm long by 10.2 cm wide by 3.1 cm tall. A central, test fluid well 9008 has a circular opening of 25 mm in diameter is located at the top plane of the plate with initial lateral walls that extend 15 mm deep at a 90° angle and then slope downward at an angle of 82° for an additional depth of 7.5 mm to reach the test fluid reservoir 9003. The test fluid reservoir 9003 is concentric to the test fluid well 9008 and has a diameter of 6.6 mm with lateral walls that extend 5 mm deep at a 90° angle. The test fluid reservoir 9003 opens into the longitudinal fluid channel 9007 located at the bottom of the plate. The longitudinal fluid channel 9007 has lateral walls that initially extend 3.5 mm deep at the midpoint of the channel (just beneath the test fluid reservoir 9003), then slant downward at an angle 9007a of 0.72° towards each longitudinal end of the channel to a final depth of 3 mm. The longitudinal fluid channel opens to the bottom plane of the plate for the fluid to be introduced onto the underlying test sample. The longitudinal fluid channel 9007 is centered over the test fluid reservoir 9003 and extends in a direction that is perpendicular to the electrodes 9004. The longitudinal fluid channel 9007 has a width of 5 mm and a length of 80 mm, with lateral edges that are rounded with a radius 9007b of 1.0 mm. The longitudinal ends of the longitudinal fluid channel 9007 are rounded with a radius 9009 of 2.5 mm. Two wells 9002 (80.5 mm long by 24.5 mm wide by 25 mm deep) located outboard of the fluid reservoir, are filled with stainless steel shot (or equivalent) to adjust the overall mass of the plate to provide a constraining pressure of 0.10 psi (7.0 g/cm²) to the Test Area. The procedure for determining the test area is subsequently described herein. Electrodes 9004 are embedded in the plate 9001, connecting the exterior banana jacks 9006 to the inside wall 9005 of the longitudinal fluid channel 9003. A circuit interval timer is plugged into the jacks 9006, monitors the impedance between the two electrodes 9004, and measures the time from introduction of the nAMF into reservoir 9003 until the nAMF drains from the reservoir. The timer has a resolution of 0.01 sec.

A pretreatment plate is used in combination with a pretreatment weight to apply tiny droplets of nAMF to the surface of the test sample as a means to prime the surface of the test sample prior to the introduction of the full liquid dose. The pretreatment plate is constructed of Plexiglass, or equivalent, that is 14 inch (35.6 cm) long by 8 inch (20.3 cm) wide with a thickness of about 0.25 inch (6.4 mm). The pretreatment plate has five circular markers, each 5 mm in diameter, placed 1 cm apart (center to center) that are aligned along the longitudinal axis of the plate. The central marker is centered at the lateral midpoint of the plate. These markers indicate the placement of the nAMF droplets. The markers are located on the underside of the pretreatment plate and can be milled out or simply drawn on with a permanent marker, or equivalent. The pretreatment weight is 10.2 cm×10.2 cm and consists of a flat, smooth rigid material (e.g., stainless steel) with an optional handle. The pretreatment weight (including optional handle) has a total mass of 726 g±0.5 g to give a pressure of 0.10 psi (7.0 g/cm²) across the bottom surface area of the weight.

When measuring the interfacial fluid amounts, a rubber pad is used to provide a reproducibly flat surface that enables even pressure distribution. The IFF rubber pad is constructed from high strength neoprene rubber with 40A durometer and a thickness of ⅛ inch (available from W.W. Grainger, Inc, item #1DUV4, or equivalent) and cut to dimensions of 6 inch (15.2 cm) by 6 inch (15.2 cm).

For the overall rewet portion of the test, a padded weight assembly that applies 0.5 psi (35.1 g/cm²) to the Test Area is required. The procedure for determining the test area is subsequently described herein. The rewet weight is constructed as follows. Lay a piece of polyethylene film (about 25 microns thick, any convenient source) horizontally flat on a rigid bench surface. A piece of polyurethane foam (25 mm thick, density of 1.0 lb/ft³, IDL 24 psi, available from Concord-Renn Co. Cincinnati, OH, or equivalent) is cut to 10.2 cm by 10.2 cm and then laid centered on top of the film. A piece of Plexiglas (10.2 cm by 10.2 cm and about 6.4 mm thick) is then stacked on top of the polyurethane foam. Next the polyethylene film is used to wrap the polyurethane foam and Plexiglas plate securing it with transparent tape. A metal weight with handle is stacked on top of, and fastened to, the Plexiglass plate such that the total mass of the padded weight assembly can be adjusted to apply a pressure of 0.5 psi (35.1 g/cm²) to the Test Area.

For the IFF, SFF and overall rewet steps, various layers of filter paper are required. The filter paper is conditioned at 23° C.±2° C. and 50%±2% relative humidity for at least 2 hours prior to testing. A suitable filter paper has a basis weight of about 88 gsm, a thickness of about 249 microns with an absorption rate of about 5 seconds, and is available from Ahlstrom-Munksjo (Mt. Holly Springs, PA) as grade 632, or equivalent. The filter paper has dimensions of 5 inch by 5 inch (12.7 cm by 12.7 cm). Test samples are conditioned at 23° C.±2° C. and 50%±2% relative humidity for at least 2 hours prior to testing. Test samples are removed from their outer packaging and the wrappers are opened to unfold the product, if applicable, using care not to press down or pull on the products while handling. No attempt is made to smooth out wrinkles. Tear the release paper between the wings, if applicable, and lay the sample on a horizontally flat, rigid surface with the body-side facing up (e.g., panty-side down). Determine the dose location as follows. For symmetrical products (i.e., the front of the product is the same shape and size as the back of the product when laterally divided along the midpoint of the longitudinal axis of the product), the dose location is the intersection of the midpoints of the longitudinal and lateral axes of the absorbent core. For asymmetrical products (i.e., the front of the product is not the same shape and size as the back of the product when laterally divided along the midpoint of the longitudinal axis of the product), the dose location is the midpoint of the product's wings at the lateral midpoint of the absorbent core. For products that have a foam core with holes and slits either punched out or printed, the dose location is the longitudinal midpoint of the hole-punched (or hole-printed) region at the lateral midpoint of the absorbent core. Once determined, mark the dose location with a small dot using a black, fine-tip, permanent marker. If wings are present, fold them to the back of the product.

Determine the Test Area of the test sample, as follows. This area will be used so that the mass of the strikethrough plate and the mass of the rewet weight can be properly adjusted to deliver the required pressure (0.1 psi and 0.5 psi, respectively). Measure the width of the absorbent core of the test sample as the distance between one lateral edge of the core to the other lateral edge of the core along a line that is positioned at the dosing location and runs perpendicular to the longitudinal axis of the test sample, and record as core width to the nearest 0.01 cm. Now multiply the core width by 10.2 cm (the length of the strikethrough plate and rewet weight) and record as Test Area to the nearest 0.1 cm². The total mass of the strikethrough plate is the Test Area multiplied by 7 g/cm². The total mass of the rewet weight is the Test Area multiplied by 35.1 g/cm².

The test sample is pretreated with nAMF as follows. Place the pretreatment plate onto a horizontally flat, rigid surface such that the side with the circular markers is facing down. Using a single channel, fixed volume pipettor, accurately dispense 50 μL of nAMF onto the topside of the pretreatment plate at the location of each of the five circular markers. Position the test sample above the pretreatment plate such that the body-side of the sample is facing the plate, the longitudinal axis of the sample and plate are aligned, and the pre-marked dose location on the test sample is centered over the central droplet of nAMF on the pretreatment plate. After properly positioned, place the test sample into contact with the pretreatment plate, then immediately apply the pretreatment weight onto the back side of the test sample, centering it over the dose location/central droplet of nAMF on the pretreatment plate. Start a 40 second timer. After 40 seconds have elapsed, remove the pretreatment weight from the test sample and remove the test sample from the pretreatment plate. Invert the test sample so that the body-side is facing up, place it onto a horizontally flat, rigid surface and immediately proceed with the steps that follow.

The first acquisition time (ACQ-1) is measured as follows. Connect the electronic circuit interval timer to the strikethrough plate 9001 and zero the timer. Position the strikethrough plate 9001 above the body-side of the test sample such that the long axis of the longitudinal fluid channel 9007 on the underside of the strikethrough plate 9001 is aligned with the longitudinal axis of the test sample, and ensure that the fluid reservoir 9003 is centered over the pre-marked dose location on the test sample. To note, nAMF should be visible through the fluid reservoir 9003 at the dose location on the test sample. After properly positioned, gently place the strikethrough plate 9001 onto the test sample. Using an adjustable volume pipettor, accurately dispense 2.0 mL of nAMF into the fluid well 9008 in the strikethrough plate 9001. The fluid is dispensed, without splashing, along the angled walls of the fluid well 9008 within a period of 3 seconds or less. Immediately after the fluid has been acquired, record the first acquisition time (ACQ-1) displayed on the circuit interval timer to the nearest 0.1 seconds. Leave the strikethrough plate 9001 in place on the test sample, and immediately start a 2 minute timer.

After 2 minutes have elapsed, measure the first Interfacial Free Fluid (IFF-1) as follows. Place the IFF rubber pad onto a horizontally flat, rigid surface. Measure the mass of one layer of filter paper to the nearest 0.0001 g and record as IFF-1ᵢₙᵢₜᵢₐₗ. Place the filter paper centered onto the IFF rubber pad. Transfer the strikethrough plate 9001 from the test sample to the pre-weighed filter paper such that the plate is centered on the filter paper, and immediately start an 8 minute timer. After 10 seconds have elapsed on the 8 minute timer, remove the strikethrough plate from the filter paper and gently replace it back onto the test sample, exactly as previously positioned. Within the next 10 seconds, measure the mass of the filter paper to the nearest 0.0001 g and record as IFF-1_{final}. The second acquisition time (ACQ-2) is measured as follows. After 8 minutes have elapsed, apply the second gush of fluid using an adjustable volume pipettor to accurately dispense 4.0 mL of nAMF into the fluid well 9008 in the strikethrough plate 9001, as previously described. Immediately after the fluid has been acquired, record the second acquisition time (ACQ-2) displayed on the circuit interval timer to the nearest 0.1 second. Leave the strikethrough plate 9001 in place on the test sample, and immediately start a 2 minute timer.

After 2 minutes have elapsed, measure the second Interfacial Free Fluid (IFF-2) as follows. Place the IFF rubber pad onto a horizontally flat, rigid surface. Measure the mass of a fresh, single layer of filter paper to the nearest 0.0001 g and record as IFF-2ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and gently replace it back onto the test sample, exactly as previously positioned. Within the next 10 seconds, measure the mass of the filter paper to the nearest 0.0001 g and record as IFF-2_{final}.

The third acquisition time (ACQ-3) is measured as follows. After 8 minutes have elapsed, apply the third gush of fluid using an adjustable volume pipettor to accurately dispense 2.0 mL of nAMF into the fluid well 9008 in the strikethrough plate 9001, as previously described. Immediately after the fluid has been acquired, record the third acquisition time (ACQ-3) displayed on the circuit interval timer to the nearest 0.1 second. Leave the strikethrough plate 9001 in place on the test sample, and immediately start a 2 minute timer.

After 2 minutes have elapsed, measure the third Interfacial Free Fluid (IFF-3) as follows. Place the IFF rubber pad onto a horizontally flat, rigid surface. Measure the mass of a fresh, single layer of filter paper to the nearest 0.0001 g and record as IFF-3ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and set it on its side so that the pad-side of the plate is not contacting the bench. Within the next 10 seconds, measure the mass of the filter paper to the nearest 0.0001 g and record as IFF-3_{final}.

Measure Surface Free Fluid (SFF) as follows. After 8 minutes have elapsed, measure the mass of a fresh stack of 5 filter papers to the nearest 0.0001 g and record as SFFᵢₙᵢₜᵢₐₗ. Place the stack of filter papers on top of the body-side of the test sample such that they are centered over the dose location. Now gently place the strikethrough plate 9001 on top of the filter papers such that the pad-side of the plate is centered on the filter papers, and immediately start a 10 second timer. After 10 seconds have elapsed, remove the strikethrough plate 9001 from the filter papers and set it aside. Measure the mass of the stack of 5 filter papers to the nearest 0.0001 g and record as SFF_{final}. Immediately proceed to the next step.

Measure overall rewet as follows. Measure the mass of a fresh stack of 5 filter papers to the nearest 0.0001 g and record as REWETᵢₙᵢₜᵢₐₗ. Place the filter papers on top of the body-side of the test sample such that they are centered over the dose location. Now place the padded Rewet Weight on top of the stack of filter papers such that the weight is centered on the filter paper stack, and immediately start a 30 second timer. After 30 seconds have elapsed, remove the rewet weight and measure the mass of the stack of 5 filter papers to the nearest 0.0001 g, then record as REWET_{final}. Discard the sample and thoroughly clean and then dry the fluid well 9008, fluid reservoir 9003, longitudinal fluid channel 9007 and the bottom surface of the strikethrough plate 9001 prior to testing the next sample.

Make the following calculations for each of the parameters measured, as follows. Calculate Total Gush Absorbency Time as the sum of ACQ-1, ACQ-2 and ACQ-3, and record to the nearest 0.1 second. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate Total IFF as the sum of IFF-1, IFF-2 and IFF-3, and record to the nearest 0.1 g. Calculate SFF by subtracting SFFᵢₙᵢₜᵢₐₗ from SFF_{final}, and record to the nearest 0.0001 g. Calculate Total IFF+SFF as the sum of Total IFF and SFF, and record to the nearest 0.1 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the arithmetic mean of the three individually recorded measurements for each Acquisition Time (ACQ-1, ACQ-2 and ACQ-3) to the nearest 0.1 seconds, Total Gush Absorbency Time to the nearest 0.1 seconds, Interfacial Free Fluid (IFF-1, IFF-2 and IFF-3) to the nearest 0.0001 g, Total IFF to the nearest 0.1 g, Surface Free Fluid (SFF) to the nearest 0.0001 g, Total IFF+SFF to the nearest 0.1 g, and Overall Rewet to the nearest 0.0001 g.

New Artificial Menstrual Fluid (nAMF) Preparation

This formulation of new Artificial Menstrual Fluid (nAMF) is composed of a mixture of defibrinated sheep blood, a phosphate buffered saline solution and a mucous component. The nAMF is prepared such that it has a viscosity between 7.40 to 9.00 centipoise at 23° C.

Viscosity of the nAMF is performed using a low viscosity rotary viscometer (a suitable instrument is the Brookfield DV2T fitted with a Brookfield UL adapter, available from AMETEK Brookfield, Middleboro, MA, or equivalent). The appropriate size spindle for the viscosity range is selected, and the instrument is operated and calibrated as per the manufacturer. Measurements are taken at 23° C.±1° C. and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, sterilized, available from American Laboratories, Inc., Omaha, NE, or equivalent), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare about 200 mL of phosphate buffered saline solution, add 49.50 g±0.10 g of Solution A and 157.50 g±0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, NC, or equivalent. After the final mixing step, measure and record the viscosity of the mucous component to the nearest 0.01 centipoise at 23° C.±1° C. and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1° C. To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1° C. and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1° C. Any unused portion is discarded after testing is complete.

### Hysteresis Method

The cyclic tensile and recovery response of a test specimen is measured for two cycles of load application (“elongation”) and load removal (“recovery”) using a universal constant rate of extension test frame. Test specimens are cycled two times to two different elongations (either 50% strain or 100% strain), then back to zero strain. For each elongation, percent set (i.e., strain at the start of the second cycle), stiffness and load at yield are calculated and reported. Several different pull directions are analyzed, including MD (machine direction; long side of the specimen is parallel to the longitudinal axis of the material as it would be present in an absorbent article) and a pull where the long side of the specimen is orthogonal to the lines of activation that would be present on the material in an absorbent article, OD (orthogonal direction). All measurements are performed in a laboratory maintained at 23° C.±2° C. and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 40 mm wide. The fixtures are installed on the universal test frame and mounted such that they are horizontally and vertically aligned with one another.

Test specimens of a backsheet film test material are prepared from a layer obtained from either roll stock raw material or from a layer excised from an absorbent article test sample. If the source of the test specimen is an absorbent article, the backsheet film is isolated from all other layers of the absorbent article as follows. The absorbent article test sample is unfolded and, if present, the wings are opened. The wrapper and any additional protective layer (i.e., release paper) that covers the panty fastening adhesive are removed and a light dusting of talc is applied to the exposed adhesive to mitigate tackiness. The backsheet film is then removed from the absorbent article, exercising caution during the removal process so as to prevent any stretching or other distortion of the excised material layer. Test specimens are prepared from the backsheet film material layer (from roll stock or an absorbent article) for both the 50% elongation test and the 100% elongation test as follows. Test specimens are cut from a region of the backsheet film layer that is free of any residua of folds or unintentionally formed wrinkles. Unintentionally formed wrinkles are those that are not a direct result of the activation process. The MD test specimen is 150 mm long (parallel to the longitudinal axis of the of the material as it would be present in an absorbent article absorbent article) and 25.4 mm wide. The OD test specimen is 150 mm long (orthogonal to the lines of activation that would be present on the material in an absorbent article) and 25.4 mm wide. In like fashion, a total of six replicate test specimens for each pull direction (MD and OD) are prepared (three for the 50% elongation test and three for the 100% elongation test).

The universal test frame is prepared for a 50% elongation test as follows. The initial grip to grip separation distance is set to a nominal gage length (Lₙₒₘᵢₙₐₗ) of 100 mm and the crosshead is then zeroed. The test frame is programmed to move the grips closer together by an intentional slack of 1 mm to ensure no pretension force exists on the test specimen at the onset of the test. (During this motion, the specimen will become slack between the tensile grips.) Next, the grips are programmed to move apart at a slack speed of 1 mm/s until the slack preload of 0.1 N is exceeded. At this point, the following are true: 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). At this position the engineering strain is zero, 0.0. Engineering strain is calculated as the change in length (ΔL) divided by the initial length (L₀). Engineering strain=ΔL/L₀. For one test cycle, the grips move apart at the initial speed of 10 mm/s until the engineering strain endpoint of 0.50 mm/mm is reached, immediately followed by the grips moving toward each other at the initial speed of 10 mm/s until the crosshead signal becomes less than the crosshead return position of 0 mm. The grips remain at the 0 mm position for a hold time of 60 seconds, the then the test cycle is repeated for one additional cycle.

For the 100% elongation test, the universal test frame is programmed exactly as described for the 50% elongation test, with the exception that the strain endpoint is set to 1.00 mm/mm.

The test is executed by inserting the test specimen into the grips such that the long axis of the specimen is parallel and centered with the motion of the crosshead. The test is started and time, force and displacement data are continuously collected at a data acquisition rate of 100 Hz, and the pull direction (MD and OD) of the test specimen is noted.

Graphs depicting force/width (N/mm) vs strain (mm/mm) are constructed for the 50% elongation and 100% elongation tests for each pull direction. The strain of the test specimen at the beginning of the second cycle is defined as the strain when the slack preload of 0.1 N is exceeded for that cycle (during the loading portion of the cycle), and is recorded as set strain to the nearest 0.01 mm/mm. The set strain is then multiplied by 100 and recorded as MD % Set at 50% Elongation, OD % Set at 50% Elongation, MD % Set at 100% Elongation, and OD % Set at 100% Elongation, all to the nearest 0.1 percent. In like fashion, the overall procedure is now repeated for all three replicates for each pull direction.

Referring to the first cycle on the force/width (N/mm) vs strain (mm/mm) graphs, a secant stiffness is calculated as the slope between Point 1 and Point 2 on the loading portion of the cycle for each of the pull directions for both the 50% elongation and 100% elongation tests. For the purposes herein, Point 1 is defined as the point at zero strain on the loading portion of the first cycle, and zero strain occurs when the slack preload of 0.1 N is exceeded. Point 2 occurs at a distance that is 0.03 mm/mm away from Point 1. The secant stiffness is calculated and reported as MD Stiffness at 50% Elongation, OD Stiffness at 50% Elongation, MD Stiffness at 100% Elongation, and OD Stiffness at 100% Elongation, all to the nearest 0.01 N/mm. In like fashion, the overall procedure is now repeated for all three replicates for each pull direction.

Graphs depicting load (N) versus displacement for both cycles are constructed for the 50% elongation and 100% elongation tests for each pull direction. For the first cycle, record the peak load to the nearest 0.01 N. The energy to peak (Eₘₐₓ) is calculated as the area under the load versus displacement curve from the cycle start to the strain endpoint (0.50 mm/mm for the 50% elongation test, and 1.00 mm/mm for the 100% elongation test; during the loading portion of the cycle) and recorded as Eₘₐₓ to the nearest 0.01 N*mm. The recovered energy (E_{recovered}) is calculated as the area under the load versus displacement curve from the strain endpoint (0.50 mm/mm for the 50% elongation test and 1.00 mm/mm for the 100% elongation test) to the crosshead return of 0 mm (during the unloading portion of the cycle) and recorded as E_{recovered} to the nearest 0.01 N*mm. The percent energy recovered is calculated by dividing E_{recovered} by Eₘₐₓ then multiplying by 100 and reported as MD % Energy Recovered at 50% Elongation, OD % Energy Recovered at 50% Elongation, MD % Energy Recovered at 100% Elongation, and OD % Energy Recovered at 100% Elongation, all to the nearest 0.1 percent. In like fashion, the overall procedure is now repeated for all three replicates for each pull direction.

A graph depicting force (N) versus engineering strain for the first cycle is constructed for the 50% elongation and 100% elongation tests for each pull direction, where engineering strain was previously defined as ΔL/L₀. The yield point is the point where the force versus engineering strain curve begins to deviate from linearity. The yield point occurs when the slope of the force versus strain curve becomes zero, however a zero slope it not always exhibited by a given material. In cases where there is not a zero slope, the yield point is often “eyeballed” as the point when the curve begins to decrease in slope. A common mathematical way to define the yield point is the 2% offset method, as follows. The slope of the linear portion on the force versus strain curve is calculated and recorded as slope to the nearest 0.01 N/mm. The offset yield point is the intersection of the force versus strain curve and a line that is parallel to the slope, but offset by a strain amount (2%). Record the force value at the offset yield point for the first cycle and record as MD Yield Force, and OD Yield Force, all to the nearest 0.01 N.

The arithmetic mean among the three replicate test specimens for each pull direction and each elongation test is calculated for each of the recorded parameters and reported as MD % Set at 50% Elongation, and OD % Set at 50% Elongation, MD % Set at 100% Elongation, and OD % Set at 100% Elongation, all to the nearest 0.1 percent; MD Stiffness at 50% Elongation, OD Stiffness at 50% Elongation, MD Stiffness at 100% Elongation, and OD Stiffness at 100% Elongation, all to the nearest 0.01 N/mm; and MD % Energy Recovered at 50% Elongation, OD % Energy Recovered at 50% Elongation, MD % Energy Recovered at 100% Elongation, and OD % Energy Recovered at 100% Elongation, all to the nearest 0.1 percent.

### Simple Tensile Method

The tensile properties of a test specimen obtained from an absorbent article test sample is measured as it is pulled to 200% strain using a universal constant rate of extension test frame. Several different pull directions are analyzed, including MD (machine direction; long side of the specimen is parallel to the longitudinal axis of the absorbent article) and a pull where the long side of the specimen is orthogonal to the lines of activation on the absorbent article, OD (orthogonal direction). All measurements are performed in a laboratory maintained at 23° C.±2° C. and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 35 mm wide. The fixtures are installed on the universal test frame and mounted such that they are horizontally and vertically aligned with one another.

Measurements are made on two different types of test specimens excised from absorbent article test samples. One type of test specimen comprises all of the layers of the absorbent article, from the topsheet to the backsheet film and, for the purposes of this test method, this type of test specimen is herein referred to as a full product test specimen. The other type of test specimen comprises the backsheet film material excised from the absorbent article test sample and, for the purposes of this test method, this type of test specimen is herein referred to as a backsheet film test specimen. When excising the test specimens from an absorbent article, care is used to prevent contamination or distortion to the material layers within the test sample and specimen during the process. The test specimens are prepared as follows. The absorbent article test sample is unfolded and, if present, the wings are opened. The wrapper and any additional protective layer (i.e., release paper) that covers the panty fastening adhesive are removed and a light dusting of talc is applied to the exposed adhesive to mitigate tackiness. Test specimens are cut from a region of the absorbent article that is free of any residua of folds or unintentionally formed wrinkles. Unintentionally formed wrinkles are those that are not a direct result of the activation process. The full product test samples require no further preparation except for cutting the test specimens into the appropriately oriented directions as follows. The full product MD test specimen is 100 mm long (parallel to the longitudinal axis of the absorbent article test sample) and 25.4 mm wide. The full product OD test specimen is 100 mm long (orthogonal to the lines of activation on the absorbent article) and 25.4 mm wide. In like fashion, a total of three replicate full product test specimens for each pull direction (MD and OD) are prepared. The backsheet film test specimens are obtained from absorbent article test samples that have had all of the layers removed in order to isolate the backsheet film. Caution is exercised during the removal of layers so as to prevent any stretching or other distortion of the backsheet film during the process. Once the backsheet film is isolated, the backsheet film MD and OD test specimens (100 mm long by 25.4 mm wide) are cut according to the same orientations as described for the full product test specimens. A total of three replicate backsheet film test specimens for each pull direction (MD and OD) are prepared.

The universal test frame is prepared for a constant rate of extension tensile test to 200% strain, with slack compensation and gage length adjustment as follows. The initial grip to grip separation distance is set to a nominal gage length (Lₙₒₘᵢₙₐₗ) of 50 mm and crosshead is zeroed. The test frame is programmed to move the grips closer together by an intentional slack of 5 mm to ensure no pretension force exists on the test specimen at the onset of the test. (During this motion, the specimen will become slack between the tensile grips.) Next, the grips are programmed to move apart at a slack speed of 5 mm/s until the slack preload of 0.10 N is exceeded. At this point, the following are true: 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). At this position the engineering strain is zero, 0.0. Engineering strain is calculated as the change in length (ΔL) divided by the initial length (L₀). Engineering strain=ΔL/L₀. The grips are then programmed to move apart at a test speed of 5.0 mm/s until the crosshead displacement of 100 mm is reached (200% strain). The grips then return to the nominal gage length.

The test is executed by inserting the test specimen into the grips such that the long axis of the specimen is parallel and centered with the motion of the crosshead. The test is started and time, force and extension (ΔL) data is continuously collected at a data acquisition rate of 20 Hz. In like fashion, the test is executed for all of the prepared test specimens (full product and backsheet film).

A graph depicting force (N) versus extension, ΔL (mm) is constructed for each specimen type (full product and backsheet film) for each pull direction. For the MD full product test specimens, record the force values to the nearest 0.1 N at the following extensions: 5 mm, 10 mm, 15 mm, and 20 mm. Calculate the arithmetic mean of the force value at each extension across all three MD full product test specimen replicates, and report as MD Full Product Force Threshold at each extension to the nearest 0.1 N. In like fashion, record the force values to the nearest 0.1 N at 5 mm, 10 mm, 15 mm, and 20 mm extensions and then calculate the arithmetic mean of the force value at each extension across all three OD full product test specimen replicates, and report as OD Full Product Force Threshold, respectively, to the nearest 0.1 N. In like fashion, record the force values at extensions 5 mm, 10 mm, 15 mm, and 20 mm for all of the MD and OD backsheet film test specimen replicates and record to the nearest 0.1 N. Calculate the arithmetic mean across all three backsheet film test specimen replicates for the force value at each extension for each of the pull directions, and report as MD Backsheet Film Force Threshold and OD Backsheet Film Force Threshold, all to the nearest 0.1 N for each extension.

A graph depicting force (N) versus engineering strain (%) is constructed for each specimen type and for each pull direction, where engineering strain was previously defined as ΔL/L₀. The array of engineering strain values are converted from units of mm/mm to units of percent by multiplying by 100. The yield point is the point where the force versus engineering strain curve begins to deviate from linearity. The yield point occurs when the slope of the force versus strain curve becomes zero, however a zero slope it not always exhibited by a given material. In cases where there is not a zero slope, the yield point is often “eyeballed” as the point when the curve begins to decrease in slope. A common mathematical way to define the yield point is the 2% offset method, as follows. The slope of the linear portion on the force versus strain curve is calculated and recorded as slope to the nearest 0.01 N/mm. The offset yield point is the intersection of the force versus strain curve and a line that is parallel to the slope, but offset by a strain amount (2%). Record the force value at the offset yield point for all replicates for each specimen type and each pull direction as MD full product yield force, OD full product yield force, MD backsheet film yield force, OD backsheet film yield force, all to the nearest 0.01 N. Now determine the percent strain that corresponds to each yield force value for all replicates for each specimen type and each pull direction and record as MD full product yield strain, OD full product yield strain, MD backsheet film yield strain, and OD backsheet film yield strain, all to the nearest 0.1%. For purposes herein, the term modulus will be defined as the yield force (N) divided by the yield strain (%). The modulus is calculated by dividing the yield force by the corresponding yield strain for all replicates for each specimen type and each pull direction, and recorded as MD full product modulus, OD full product modulus, MD backsheet film modulus, and OD backsheet film modulus, all to the nearest 0.01 N/%. The arithmetic mean of the yield force values across the replicates within each specimen type and each pull direction is calculated and reported as MD Full Product Yield Force, OD Full Product Yield Force, MD Backsheet Film Yield Force, and OD Backsheet Film Yield Force, all to the nearest 0.01 N. The arithmetic mean of the modulus values across the replicates within each specimen type and each pull direction is calculated and reported as MD Full Product Modulus, OD Full Product Modulus, MD Backsheet Film Modulus, and OD Backsheet Film Modulus, all to the nearest 0.01 N/%.

### SEM Imaging Method

A Scanning Electron Microscope (SEM) is used to obtain images of the cross-section of a test specimen in its relaxed state to enable visualization of the microstructure of an absorbent article, including surface features such as micro and macro wrinkles, texture, and spacing between the discrete foam pieces created by the activation process. Quantitative measures of the gap between the foam pieces are made using image analysis, and qualitative assessments are also used to describe the microstructure of the backsheet film.

Secondary Electron (SE) images are obtained using an SEM such as the FEI Quanta 450 (available from FEI Company, Hillsboro, OR), or equivalent. The instrument is calibrated according to the manufacturer's instructions prior to use to ensure an accurate distance scale.

Absorbent article samples are conditioned in a laboratory maintained at 23° C.±2° C. and 50%±2% relative humidity for at least 2 hours prior testing. A cross-sectioned test specimen is prepared by removing it from an absorbent article as follows. A test sample is taken from an area on the absorbent article that excludes all residua of folds and also excludes regions where panty fastening adhesive is present. While preparing the test sample and subsequent specimen, care is used to prevent any contamination, stretching, or other distortion to the region that will be analyzed. A cross-sectioned specimen that comprises all layers of the absorbent article (from the topsheet to the backsheet film) is prepared by sectioning an edge of the test sample that is orthogonal to the activation lines with a fresh, new razor blade (such as VWR Single Edge Industrial Razor blade No. 9, surgical carbon steel, or equivalent). To achieve a very clean cut edge, the test specimen can be sectioned after exposing the test sample to liquid nitrogen. The topsheet (body-facing) side of the test specimen is adhered to a SEM mount using double-sided conductive tape (such as Cu, 3M available from electron microscopy sciences, or equivalent), such that the cross-section can be viewed when the test specimen is tilted backward 90°. In order to qualitatively assess the micro wrinkles of the film layer created by the activation process, it can be beneficial to remove most of the underlying layers, including the foam, after exposing the test sample to liquid nitrogen. A cross-sectioned specimen of the film is then prepared by sectioning an edge that is orthogonal to the activation lines using a fresh, new razor blade while the film is submerged in liquid nitrogen.

High resolution SEM images (e.g., 6.8 mega pixel) of the test specimen are obtained as follows. The cross-sectioned surface of the test specimen is initially viewed at a low magnification (e.g., 15×; horizontal field width about 13 mm) to identify the gaps between the discrete foam pieces as well as the surface features of the backsheet film layer within the activation lines, and images are acquired. The cross-sectioned surface of the test specimen is then viewed at a higher magnification (e.g., at least 35×; horizontal field width about 16 mm, and at least 300×; horizontal field width about 690 microns), and images are acquired. In order to view any micro wrinkles that are present on the test specimen within the lines of activation, a significantly higher magnification (e.g., 2250×; horizontal field width about 100 microns) is used and images are acquired. The test specimen is then tilted forward about 30-40 degrees such that the outer surface of the backsheet film can be viewed, and images captured. A sufficient number of images are obtained such that multiple lines of activation can be analyzed. In like fashion, repeat the entire imaging process on a sufficient number of replicate test specimens such that a thorough representation of the microstructure of the absorbent article test sample is achieved and a minimum of ten separate lines of activation are captured in images for subsequent analysis. To note, a fresh, new razor blade is used for each cross-sectioned specimen that is prepared.

A qualitative assessment of the images enables the analyst to determine the presence of internal and external wrinkles, both micro and macro, on the backsheet film within the lines of activation, as well as other micro features such as texture on the outer surface of the film. In addition to the qualitative evaluation that the collection of images enables, simple quantitative measures are also possible using image analysis software (e.g., built into the SEM instrument, or standalone software such as ImageJ v. 1.52, National Institute of Health, USA, or equivalent). A suitable image analysis software is used to measure the distance between the discrete foam pieces formed by the activation process. Distance measurements are made on images of the cross-sectioned surface of the test specimen at the following specified locations. The distance between the discrete foam pieces is measured at a location that is about 0.5 mm from the topsheet (body-facing side) of the product at ten separate lines of activation and recorded as relaxed upper gap to the nearest 0.01 microns. The distance between the discrete foam pieces is also measured at a location that is about 0.5 mm from the backsheet (garment-facing side) of the product at ten separate lines of activation and recorded as relaxed lower gap to the nearest 0.01 microns. The arithmetic mean is calculated across all twenty measured gap values (ten relaxed upper gap values and ten relaxed lower gap values) and reported as Relaxed Gap to the nearest 0.01 microns.

### Stretched Gap Measurement Method

An activation process is used to create spacing between discrete foam pieces within an absorbent article. The gap size between neighboring foam pieces increases when tension is applied to the absorbent article in a pull direction that is orthogonal to the lines of activation within the absorbent article. The change in length after tension is applied to an absorbent article test specimen, along with the number of gaps present within said test specimen, are used to calculate the stretched gap size. The change in length of an absorbent article test specimen before and after a static weight is applied for a specified amount of time is divided by the number of gaps present in the test specimen to give stretched gap. All measurements are performed in a laboratory maintained at 23° C.±2° C. and 50%±2% relative humidity and test specimens are conditioned in this environment for at least 2 hours prior to testing.

A test specimen is excised from an absorbent article test sample such that it comprises all layers of the absorbent article, from the topsheet to the backsheet film. When excising the test specimen from an absorbent article, care is used to prevent contamination or distortion to the material layers within the test sample and specimen during the process. The test specimens are prepared as follows. The absorbent article test sample is unfolded and, if present, the wings are opened. The wrapper and any additional protective layer (i.e., release paper) that covers the panty fastening adhesive are removed and a light dusting of talc is applied to the exposed adhesive to mitigate tackiness. Test specimens are cut from a region of the absorbent article that is free of any residua of folds or unintentionally formed wrinkles. Unintentionally formed wrinkles are those that are not a direct result of the activation process. The axis of the long side of the test specimen is oriented such that it is orthogonal to the lines of activation on the absorbent article. A sharp blade is used to excise a test specimen that is 100 mm long (orthogonal to the lines of activation on the absorbent article) and 25.4 mm wide. In like fashion, a total of three replicate test specimens are prepared.

A set of lightweight (<80 g) vise action clamps with knife edge grip faces that are about 35 mm wide are used to hold the sample during the test. A 760 g weight that is capable of being affixed to the lower grip is also required, as is a calibrated steel ruler (traceable to NIST or equivalent). The upper clamp is secured to a rigid mount such that there is sufficient space beneath it to allow for the long side of the test specimen to hang and stretch freely in a vertically aligned orientation once the lower grip and 760 g weight are attached. To execute the test, the test specimen is inserted into the upper grip with its long side vertically aligned. The lower grip (without the 760 g weight) is then attached to the test specimen at a location that is about 50 mm away from the upper grip. The gage length (grip to grip separation) is then measured using the steel ruler and recorded as initial length to the nearest 0.1 mm. The number of orthogonal activation lines that are present in the test specimen between the clamped edges is counted and recorded as number of gaps. The 760 g weight is then affixed to the lower grip and the sample, lower grip, and weight are allowed to hang freely for 10 seconds. After 10 seconds have elapsed, the gage length (grip to grip separation) is measured using the steel ruler and recorded as final length to the nearest 0.1 mm. The stretched gap is calculated by subtracting the initial length from the final length and then dividing by the number of gaps. The stretched gap is recorded to the nearest 0.1 mm. In like fashion, the entire process is repeated for the remaining test specimens. The arithmetic mean of the stretched gap values measured for all three replicates is calculated and reported as Stretched Gap to the nearest 0.1 mm.

### Combinations

A. A disposable absorbent article comprising:
   a topsheet;
   a backsheet; and
   an absorbent core structure comprising an open-celled absorbent foam material disposed between the topsheet and the backsheet;
   wherein the backsheet comprises an elastic layer and a constricting layer;
wherein the absorbent foam material comprises a plurality of discrete foam pieces, wherein the discrete foam pieces are separated from neighboring pieces by a Relaxed Gap of 280 microns or less, as measured according to the SEM Imaging Method, and by a Stretched Gap of from 500 microns to 2.5 mm, as measured by the Stretched Gap Measurement Method.
B. The disposable absorbent article of Paragraph A, wherein the elastic layer has two surfaces and a skin on at least one of the surfaces.
C. The disposable absorbent article of Paragraphs A or B, wherein the elastic layer comprises an elastically extensible material selected from the group consisting of styrene-isoprene-styrene block copolymers; styrene-butadiene-styrene block copolymers; styrene ethylene-butylene-styrene block copolymers; polyurethanes; ethylene copolymers; polyether block amides; and combinations thereof.
D. The disposable absorbent article of any of Paragraphs A-C, wherein the constricting layer comprises polyolefin.
E. The disposable absorbent article of Paragraph D, wherein the constricting layer comprises polyethylene.
F. The disposable absorbent article of any of Paragraphs A-E, wherein the constricting layer and the elastic layer are co-extruded.
G. The disposable absorbent article of any of Paragraphs A-E, wherein the backsheet is a laminate.
H. The disposable absorbent article of any of Paragraphs A-G, wherein from 5 gsm to 15 gsm of an adhesive is positioned between a garment-facing surface of the topsheet and a wearer-facing surface of the discrete foam pieces and bonds the discrete foam pieces to the topsheet.
I. The disposable absorbent article of any of Paragraphs A-H, further comprising from 5 gsm to 15 gsm of an adhesive positioned between a wearer-facing surface of the backsheet and a garment-facing surface of the discrete foam pieces, wherein the adhesive bonds the discrete foam pieces to the backsheet.
J. The disposable absorbent article of any of Paragraphs A-I, wherein the discrete foam pieces comprise a high internal phase emulsion foam.
K. The disposable absorbent article of any of Paragraphs A-J, wherein the topsheet and the backsheet each comprises a first plurality of lines of deformation extending in a first direction and a second plurality of lines of deformation extending in a second direction; wherein the absorbent core structure comprises a plurality of discrete foam pieces arranged along the lines of deformation; wherein at least a portion of the backsheet that is located in the first and second plurality of lines of deformation has a plurality of micro-wrinkles.
L. The disposable absorbent article of Paragraph K, wherein the first plurality of lines of deformation form an angle α with respect to the longitudinal axis and the second plurality of lines of deformation form an angle β with respect to the longitudinal axis, wherein the angle α and the angle β are each from 5 degrees to 85 degrees.
M. The disposable absorbent article of Paragraph K, wherein the portion of the backsheet that is located in the first and second plurality of lines of deformation further comprise one or more macro-wrinkles.
N. The disposable absorbent article of any of Paragraphs A-M, wherein the backsheet has an MD % Set at 100% Elongation of from 1% to 35%, as measured by the Hysteresis Method.
O. The disposable absorbent article of any of Paragraphs A-N, wherein the article exhibits an OD Full Product Force Threshold of at least 7.2 N at 15 mm of extension, as measured according to the Simple Tensile Method.
P. The disposable absorbent article of any of Paragraphs A-O, wherein the constricting layer is on a garment-facing surface of the elastic layer.
Q. The disposable absorbent article of any of Paragraphs A-P, wherein the absorbent article further comprises a panty fastening adhesive disposed on a garment facing surface of the backsheet.
R. The disposable absorbent article of any of Paragraphs A-Q, wherein the backsheet has an OD % Energy Recovered at 50% Elongation of between 25% and 35%.
A1. A disposable absorbent article comprising:
   a topsheet;
   a backsheet comprising an elastic layer;
   a panty fastening adhesive disposed on a garment facing surface of the backsheet; and
   an absorbent core structure comprising an open-celled absorbent foam material disposed between the topsheet and the backsheet;
   wherein the absorbent foam material comprises a plurality of discrete foam pieces, wherein the discrete foam pieces are separated from neighboring pieces by a Relaxed Gap 280 microns or less; and
   wherein the article has an OD Full Product Force Threshold at 15 mm between 3.6 N and 7.2 N and an OD % Energy Recovered at 50% Elongation greater than 35%.
A2. The disposable absorbent article of Paragraph A1, wherein the absorbent article comprises from 9 gsm to 22 of the panty fastening adhesive.
A3. The disposable absorbent article of Paragraph A1 or A2, wherein from about 5 gsm to about 15 gsm of an adhesive is positioned between a garment-facing surface of the topsheet and a wearer-facing surface of the discrete foam pieces and bonds the discrete foam pieces to the topsheet.
A4. The disposable absorbent article of any of Paragraphs A1-A3, further comprising from 5 gsm to 15 gsm of an adhesive positioned between a wearer-facing surface of the backsheet and a garment-facing surface of the discrete foam pieces, wherein the adhesive bonds the discrete foam pieces to the backsheet.
A5. The disposable absorbent article of any of Paragraphs A1-A4, wherein the discrete foam pieces comprise a high internal phase emulsion foam.
A6. The disposable absorbent article of any of Paragraphs A1-A5, wherein the topsheet and the backsheet each comprises a first plurality of lines of deformation extending in a first direction and a second plurality of lines of deformation extending in a second direction; wherein the absorbent core structure comprises a plurality of discrete foam pieces arranged along the lines of deformation; wherein at least a portion of the backsheet that is located in the first and second plurality of lines of deformation has a plurality of micro-wrinkles.
A7. The disposable absorbent article of Paragraph A6, wherein the first plurality of lines of deformation form an angle α with respect to the longitudinal axis and the second plurality of lines of deformation form an angle β with respect to the longitudinal axis, wherein the angle α and the angle β are each from 5 degrees to 85 degrees.
A8. The disposable absorbent article of Paragraph A6, wherein the portion of the backsheet that is located in the first and second plurality of lines of deformation further comprise one or more macro-wrinkles.
A9. The disposable absorbent article of any of Paragraphs A1-A8, wherein the backsheet has an MD % Set at 100% Elongation of from 1% to 35%, as measured by the Hysteresis Method.
A10. The disposable absorbent article of any of Paragraphs A1-A9, wherein the elastic layer comprises an elastically extensible material selected from the group consisting of styrene-isoprene-styrene block copolymers; styrene-butadiene-styrene block copolymers; styrene ethylene-butylene-styrene block copolymers; polyurethanes; ethylene copolymers; polyether block amides; and combinations thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “approximately 40 mm.”

Where ranges of measured quantities or property values are described and/or recited as characterizations of subject matter contemplated herein, such ranges are deemed to include and contemplate any and all sub-ranges within the ranges.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a topsheet;
a backsheet; and
an absorbent core structure comprising an open-celled absorbent foam material disposed between the topsheet and the backsheet;
wherein the backsheet comprises an elastic layer and a constricting layer;
wherein the absorbent foam material comprises a plurality of discrete foam pieces, wherein the discrete foam pieces are separated from neighboring pieces by a Relaxed Gap of about 280 microns or less, as measured according to the SEM Imaging Method, and by a Stretched Gap of from about 500 microns to about 2.5 mm, as measured by the Stretched Gap Measurement Method.

2. The disposable absorbent article of claim 1, wherein the elastic layer has two surfaces and a skin on at least one of the surfaces.

3. The disposable absorbent article of claim 1, wherein the elastic layer comprises an elastically extensible material selected from the group consisting of styrene-isoprene-styrene block copolymers; styrene-butadiene-styrene block copolymers; styrene ethylene-butylene-styrene block copolymers; polyurethanes; ethylene copolymers; polyether block amides; and
combinations thereof.

4. The disposable absorbent article of claim 1, wherein the constricting layer comprises polyolefin.

5. The disposable absorbent article of claim 4, wherein the constricting layer comprises polyethylene.

6. The disposable absorbent article of claim 1, wherein the constricting layer and the elastic layer are co-extruded.

7. The disposable absorbent article of claim 1, wherein from about 5 gsm to about 15 gsm of an adhesive is positioned between a garment-facing surface of the topsheet and a wearer-facing surface of the discrete foam pieces and bonds the discrete foam pieces to the topsheet.

8. The disposable absorbent article of claim 7, further comprising from about 5 gsm to about 15 gsm of an adhesive positioned between a wearer-facing surface of the backsheet and a garment-facing surface of the discrete foam pieces, wherein the adhesive bonds the discrete foam pieces to the backsheet.

9. The disposable absorbent article of claim 1, wherein the discrete foam pieces comprise a high internal phase emulsion foam.

10. A disposable absorbent article comprising:
a longitudinal axis and a lateral axis;
a topsheet;
a backsheet; and
an absorbent core structure comprising an open-celled absorbent foam material disposed between the topsheet and the backsheet;
wherein the backsheet comprises an elastic layer and a constricting layer;
wherein the topsheet and the backsheet each comprise a first plurality of lines of deformation extending in a first direction and a second plurality of lines of deformation extending in a second direction;
wherein the absorbent core structure comprises a plurality of discrete foam pieces arranged along the lines of deformation;
wherein at least a portion of the backsheet that is located in the first and second plurality of lines of deformation has a plurality of micro-wrinkles.

11. The disposable absorbent article of claim 10, wherein the first plurality of lines of deformation form an angle α with respect to the longitudinal axis and the second plurality of lines of deformation form an angle β with respect to the longitudinal axis, wherein the angle α and the angle β are each from about 5 degrees to about 85 degrees.

12. The disposable absorbent article of claim 10, wherein the portion of the backsheet that is located in the first and second plurality of lines of deformation further comprise one or more macro-wrinkles.

13. The disposable absorbent article of claim 10, wherein the backsheet has an MD % Set at 100% Elongation of from about 1% to about 35%, as measured by the Hysteresis Method.

14. The disposable absorbent article of claim 10, wherein the article exhibits an OD Full Product Force Threshold of at least 7.2 N at 15 mm of extension as measured according to the Simple Tensile Method.

15. The disposable absorbent article of claim 10, wherein the discrete foam pieces are separated from neighboring pieces by a Relaxed Gap of from about 10 microns to about 280 microns, as measured according to the SEM Imaging Method.

16. A disposable absorbent article comprising:
a topsheet;
a backsheet comprising an elastic layer and a constricting layer; and
an absorbent core structure comprising an open-celled absorbent foam material disposed between the topsheet and the backsheet;
wherein the absorbent foam material comprises a plurality of discrete foam pieces, wherein the discrete foam pieces are separated from neighboring pieces by a Relaxed Gap of from about 10 microns to about 280 microns, as measured according to the SEM Imaging Method; and
wherein the article exhibits an OD Full Product Force Threshold at 15 mm extension of greater than 7.2 N, as measured according to the Simple Tensile Method.

17. The disposable absorbent article of claim 16, wherein the backsheet has an OD % Energy Recovered at 50% Elongation of between about 25% and about 35%.

18. The disposable absorbent article of claim 16, wherein the constricting layer is on a garment-facing surface of the elastic layer.

19. The disposable absorbent article of claim 18, wherein the absorbent article further comprises a panty fastening adhesive disposed on a garment facing surface of the backsheet.

20. The disposable absorbent article of claim 18, wherein the absorbent article comprises from about 9 gsm to about 22 of the panty fastening adhesive.
